(19) United States
(12) Patent Application Publication
(10) Pub. No.: US 2026/0166045 A1
(43) Pub. Date: Jun. 18, 2026

(54) COMBINATION THERAPY COMPRISING GSPT1-DIRECTED MOLECULAR GLUE DEGRADERS AND PI3K/AKT/mTOR PATHWAY INHIBITORS

(71) Applicant: Monte Rosa Therapeutics, Inc., Boston, MA (US)

(72) Inventors: Ralph Tiedt, Boston, MA (US); Agustin Chicas, Boston, MA (US); Michael Song, Boston, MA (US); Gerald Gavory, Boston, MA (US); Silvia Buonamici, Boston, MA (US)

(21) Appl. No.: 19/016,780

(22) Filed: Jan. 10, 2025

Related U.S. Application Data
(63) Continuation of application No. PCT/US2023/070048, filed on Jul. 12, 2023.
(60) Provisional application No. 63/388,992, filed on Jul. 13, 2022.

(51) Int. Cl. A61K 31/5377; C07D 401/14

(52) U.S. Cl. CPC A61K 31/5377; C07D 401/14

(57) ABSTRACT

This disclosure relates to combination therapies for treatment of cancer employing a compound that causes degradation of Eukaryotic peptide chain release factor GTP-binding subunit eRF3A (GSPT1) in combination with a compound that inhibits the PI3K/Akt/mTOR pathway.

### CROSS-REFERENCE TO RELATED APPLICATION

This application is a continuation of International Patent Application No. PCT/US2023/070048, filed Jul. 12, 2023, which claims the benefit of U.S. Application Ser. No. 63/388,992, filed on Jul. 13, 2022, which is incorporated herein by reference in its entirety.

### REFERENCE TO SEQUENCE LISTING

This application contains a Sequence Listing which has been submitted electronically in XML format. The Sequence Listing XML is incorporated herein by reference. Said XML file, created on Jan. 10, 2025, is named MRT-162USWOC1_SL.xml and is 3,076 bytes in size.

### TECHNICAL FIELD

This disclosure relates to combination therapies for treatment of cancer employing a compound that causes degradation of Eukaryotic peptide chain release factor GTP-binding subunit eRF3A (GSPT1) in combination with a compound that inhibits the PI3K/Akt/mTOR pathway.

### BACKGROUND

GSPT1 is a translation termination factor that binds to eRF1, thereby enabling eRF1 to recognize termination codons and to catalyze release of the nascent polypeptide chain from tRNA, which is then followed by ribosome disassembly and recycling.

Molecular glue degraders (“MGDs”; Tan et al. Nature 2007, 446, 640-645 and Sheard et al. Nature 2010, 468, 400-405) are compounds that modulate E3 ligases (e.g., cereblon) by redirecting the activity of the E3 ligases. Molecular glue degraders mediate an alteration of the E3 ligase surface that enables an interaction between the E3 ligase and the target protein that leads to ubiquitination of the target protein and its subsequent destruction by the proteasome.

The PI3K/AKT/mTOR pathway is an intracellular signaling pathway that plays an important role in the cell cycle control. Phosphorylation of PI3K (Class I phosphoinositide 3-kinase) activates Akt (Serine-Threonine Protein Kinase or Protein Kinase B) which regulates several downstream molecules, including mTOR (Mammalian Target of Rapamycin). PI3K, Akt, and mTOR inhibitors are of interest for treating certain cancers. (Hennessy et al. Nature Reviews Drug Discovery 2005, 4, 988-1004, Vanhaesebroeck et al. Nature Reviews Drug Discovery 2021, 20, 741-769, and Liu & Sabatini. Nature Reviews Molecular Cell Biology 2020, 21, 183-203).

There are four PI3K isoforms (alpha, beta, delta and gamma) and inhibitors can be essentially selective for a single isoform or can inhibit multiple isoforms. Among the PI3K inhibitors currently approved (for monotherapy or in combination therapy) are Alpelisib/NVP-BYL719/Piqray (Novartis), Copanlisib/BAY 80-6946/Aligopa (Bayer), Idelalisib/CAL-101/GS-1101/Zydelig (Gilead), Duvelisib/IPI-145/Copiktra (Secura Bio), and Umbralisib/TGR-1202 (TG Therapeutics). Among the PI3K inhibitors said to be selective for PIK3 alpha (PIK3CA) are Serabelisib (INK-1117/TAK-117/MLN1117/ART-001/Petra 06); Alpelisib; Inavolisib (GDC-0077/RG-6114); and MEN1611 (CH5132799).

Among FDA approved mTOR inhibitors are Sirolimus, Temsirolimus, and Everolimus.

Among the Akt inhibitors that have been studied for treatment of cancer are: ipatasertib (RG7440), afuresertib (GSK2110183), uprosertib (GSK2141795), and capivasertib (AZD5363), all of which are thought to bind the ATP active site, and Perifosine (KRX-0401), which is thought be an allosteric inhibitor.

### SUMMARY

Described herein is a method of treating a patient suffering from cancer comprising administrating
(i) compound 1-1

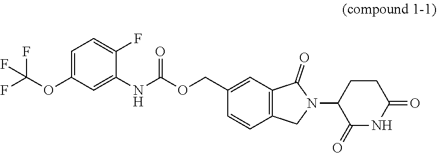 [SMILES: O=C1CCC(N2Cc3ccc(COC(=O)Nc4cc(OC(F)(F)F)ccc4F)cc3C2=O)C(=O)N1]

and
(ii) everolimus.

Also described herein is method of treating a patient suffering from cancer comprising administrating
(i) a compound or a pharmaceutically acceptable salt or stereoisomer thereof of formula Va:

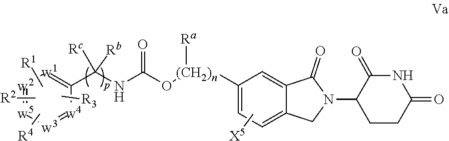

wherein
w¹, w², w³, w⁴, w⁵ are independently of each other selected from C and N, with the proviso that at least three of w, w², w³, w⁴, w⁵ are C;
X⁵ is H, linear or branched C₁₋₆ alkyl, —C₁₋₄ alkoxy, —CN, halogen, CF₃, CHF₂, CMeF₂, OCF₃, OCHF₂;
R¹, R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
R^{a} is H, linear or branched C₁₋₄ alkyl, R^{b}, R^{c} are independently of each other H, linear or branched C₁₋₄ alkyl; n is 1, or 2; and p is 0 or 1; and
(ii) everolimus.

In some embodiments, the cancer is selected from the group consisting of breast cancer, lung cancer and multiple myeloma. In some embodiments, the cancer is selected from the group consisting of breast cancer, lung cancer, lymphoma and multiple myeloma. In certain embodiments, the cancer is selected from the group consisting of breast cancer, non-small lung cancer, small lung cancer, B cell lymphoma and multiple myeloma.

In some embodiments, the cancer has elevated expression of one or more Myc transcription factor biomarkers. In certain embodiments, the one or more Myc transcription factor biomarkers are selected from the group consisting of: L-Myc, N-Myc, c-Myc, EIF4EBP1 and EIF4EBP2. In certain embodiments, the one of more Myc transcription factor biomarkers are selected from the group consisting of: L-Myc and N-Myc.

In some embodiments, the cancer exhibits a PIK3CA mutation.

Described herein is a method of treating a patient suffering from cancer comprising administrating:
(i) a GSPT1 degrader; and
(ii) a compound selected from the group consisting of: a PI3K inhibitor, an Akt inhibitor, and an mTOR inhibitor.

In some embodiments, the cancer is associated with dysregulated translation. In some embodiments, the cancer exhibits one or more PI3K-AKT-mTOR pathway gene mutations. In certain embodiments, the cancer exhibits one or more mutations selected from the group consisting of: a PIK3CA mutation, a PTEN mutation, an AKT1 mutation, a PIK3R1 mutation, and a PIK3CG mutation.

In some embodiments, the cancer exhibits a PIK3CA mutation. In some embodiments, the PIK3CA mutation is selected from the group consisting of: R38C, R38H, R88Q, P104R, G106V, R108P, de1K11, G118D, G122D, P124T, N345K, D350H, C378R, C420R, E453Q, P539R, E542K, E542G, E542V, E545K, E545G, E545D, Q546K, Q546P, Q661K, H701P, C901F, F909L, S1008P, T1025A, T1025N, M1043I, H1047Y, H1047R, H1047L, and G1049S. In certain embodiments, the PIK3CA mutation is selected from the group consisting of E542K, E545K H1047R, H1047L.

In some embodiments, the cancer is selected from the group consisting of: renal angiomyolipoma, renal cell carcinoma, subependymal giant cell astrocytoma (SEGA), breast cancer, lung cancer, pancreatic cancer, and gastrointestinal (GI) cancer. In some embodiments, the cancer is selected from the group consisting of carcinoid tumor, large cell carcinoma, uterine cancer, astrocytoma, acute myeloid leukemia, arrhythmia rhabdomyosarcoma, biliary cancer, salivary gland cancer, non-hodgkin lymphoma, B-cell lymphoma and diffuse large B-cell lymphoma. In certain embodiments, the cancer is breast cancer.

In certain embodiments, the breast cancer is an estrogen receptor positive breast cancer. In certain embodiments, the breast cancer a triple-negative breast cancer.

In certain embodiments, the cancer is lung cancer. In certain embodiments, the lung cancer is non-small cell lung cancer. In certain embodiments, the lung cancer is a small cell lung cancer.

In some embodiments, the cancer is a neuroendocrine cancer. In certain embodiments, the cancer is selected from the group consisting of lung neuroendocrine cancer and pancreatic neuroendocrine cancer.

In some embodiments, the cancer is associated with tuberous sclerosis. In certain embodiments, the cancer is selected from the group consisting of renal angiomyolipoma associated with tuberous sclerosis and subependymal giant cell astrocytoma associated with tuberous sclerosis.

In some embodiments, the cancer has elevated expression of one or more Myc transcription factor biomarkers selected from the group consisting of: L-Myc, N-Myc, c-Myc, EIF4EBP1 and EIF4EBP2.

In some embodiments, the GSPT1 degrader is a compound or a pharmaceutically acceptable salt or stereoisomer thereof of formula I:

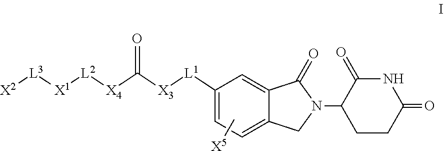

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy; or
X¹ forms together with X⁴ a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
X⁵ is H, linear or branched C₁₋₆ alkyl, —C₁₋₄ alkoxy, —CN, halogen, CF₃, CHF₂, CMeF₂, OCF₃, OCHF₂;
L¹ is a covalent bond, C₁₋₆ alkyl, which is unsubstituted or substituted with one or more of C₁₋₄ alkyl, halogen;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen.

In certain embodiments, X³ is —O—.

In some embodiments, the GSPT1 degrader is a compound or a pharmaceutically acceptable salt or stereoisomer thereof of formula II,

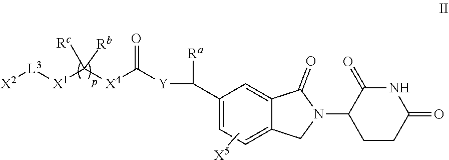

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X⁴ is —NH—;
X⁵ is H, linear or branched C₁₋₆ alkyl, —C₁₋₄ alkoxy, —CN, halogen, CF₃, CHF₂, CMeF₂, OCF₃, OCHF₂;
Y is O;
R^{a} is a H or C₁₋₄ alkyl;
R^{b}, R^{c} are independently of each other H, C₁₋₄ alkyl, preferably methyl, ethyl, or halogen, preferably F;
L³ is a covalent bond, —O—, —C₁₋₄ alkoxy or C₁₋₆ alkyl, which is unsubstituted or substituted with one or more of C₁₋₄ alkyl, halogen; and
p is 0, 1, 2.

In some embodiments, the GSPT1 degrader is a compound or a pharmaceutically acceptable salt or stereoisomer thereof of formula Va:

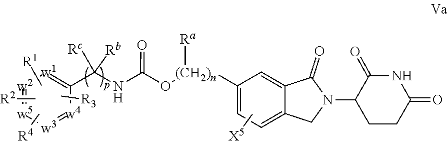

wherein
w¹, w², w³, w⁴, w⁵ are independently of each other selected from C and N, with the proviso that at least three of w¹, w², w³, w⁴, w⁵ are C;
X⁵ is H, linear or branched C₁₋₆ alkyl, —C₁₋₄ alkoxy, —CN, halogen, CF₃, CHF₂, CMeF₂, OCF₃, OCHF₂;
R¹, R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
R^{a} is H, linear or branched C₁₋₄ alkyl, R^{b}, R^{c} are independently of each other H, linear or branched C₁₋₄ alkyl; n is 1, or 2; and p is 0 or 1.

In some embodiments, the GSPT1 degrader is selected from the group consisting of
[2-(2,6-dioxopiperidin-3-yl)-3-oxo-2,3-dihydro-1H-isoindol-5-yl]methyl N-[2-fluoro-5-(trifluoromethoxy)phenyl]carbamate or a pharmaceutically acceptable salt thereof,
[2-(2,6-dioxopiperidin-3-yl)-3-oxo-2,3-dihydro-1H-isoindol-5-yl]methyl N-[4-fluoro-3-(trifluoromethoxy)phenyl]carbamate or a pharmaceutically acceptable salt thereof,
[2-(2,6-dioxopiperidin-3-yl)-3-oxo-2,3-dihydro-1H-isoindol-5-yl]methyl N-[3-(difluoromethoxy)-4-fluorophenyl]carbamate or a pharmaceutically acceptable salt thereof,
[2-(2,6-dioxopiperidin-3-yl)-3-oxo-2,3-dihydro-1H-isoindol-5-yl]methyl N-(3,5-dimethylphenyl)carbamate or a pharmaceutically acceptable salt thereof,
[2-(2,6-dioxopiperidin-3-yl)-3-oxo-2,3-dihydro-1H-isoindol-5-yl]methyl N-[3-(trifluoromethoxy)phenyl]carbamate or a pharmaceutically acceptable salt thereof,
[2-(2,6-dioxopiperidin-3-yl)-3-oxo-2,3-dihydro-1H-isoindol-5-yl]methyl N-(3-chloro-4-methylphenyl)carbamate or a pharmaceutically acceptable salt thereof, and

In some embodiments, the compound of (ii) is an mTOR inhibitor. In certain embodiments, the mTOR inhibitor is selected from the group consisting of:
(1R,9S,12S,15R,16E,18R,19R,21R,23S,24E,26E,28E,30S,32S,35R)-1,18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone or a pharmaceutically acceptable salt thereof, and
[5-[2-[(2R,6S)-2,6-dimethylmorpholin-4-yl]-4-morpholin-4-ylpyrido[2,3-d]pyrimidin-7-yl]-2-methoxyphenyl]methanol or a pharmaceutically acceptable salt thereof. In certain embodiments, the mTOR inhibitor is (1R,9S,12S,15R,16E,18R,19R,21R,23S,24E,26E,28E,30S,32S,35R)-1,18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone or a pharmaceutically acceptable salt (everolimus).

In some embodiments, the compound of (ii) is a PI3K inhibitor. In certain embodiments, the compound of (ii) is a PIK3CA inhibitor. In certain embodiments, the PIK3CA inhibitor is selected from the group consisting of:
(2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide or a pharmaceutically acceptable salt thereof,
5-[7-methyl-6-[(4-methylsulfonylpiperazin-1-yl)methyl]-4-morpholin-4-ylthieno[3,2-d]pyrimidin-2-yl]pyrimidin-2-amine or a pharmaceutically acceptable salt thereof,
ethyl 6-[5-(benzenesulfonamido)pyridin-3-yl]imidazo[1,2-a]pyridine-3-carboxylate or a pharmaceutically acceptable salt thereof, and
[6-(2-amino-1,3-benzoxazol-5-yl)imidazo[1,2-a]pyridin-3-yl]-morpholin-4-ylmethanone or a pharmaceutically acceptable salt thereof. In certain embodiments, the PIK3CA inhibitor is (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide (alpelisib).

In some embodiments, the compound of (ii) is an Akt inhibitor. In certain embodiments, the Akt inhibitor is selected from the group consisting of
4-amino-N-[(1S)-1-(4-chlorophenyl)-3-hydroxypropyl]-1-(7H-pyrrolo[2,3-d]pyrimidin-4-yl)piperidine-4-carboxamide or a pharmaceutically acceptable salt thereof, and
4-[[(1S)-2-(azetidin-1-yl)-1-[4-chloro-3-(trifluoromethyl)phenyl]ethyl]amino]quinazoline-8-carboxamide or a pharmaceutically acceptable salt thereof.

In some embodiments, the GSPT1 degrader is [2-(2,6-dioxopiperidin-3-yl)-3-oxo-2,3-dihydro-1H-isoindol-5-yl]methyl N-[2-fluoro-5-(trifluoromethoxy)phenyl]carbamate or a pharmaceutically acceptable salt thereof, and the compound of (ii) is (1R,9S,12S,15R,16E,18R,19R,21R,23S,24E,26E,28E,30S,32S,35R)-1,18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone or a pharmaceutically acceptable salt thereof.

In some embodiments, the GSPT1 degrader is [2-(2,6-dioxopiperidin-3-yl)-3-oxo-2,3-dihydro-1H-isoindol-5-yl]methyl N-[2-fluoro-5-(trifluoromethoxy)phenyl]carbamate or a pharmaceutically acceptable salt thereof, and the compound of (ii) is (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide or a pharmaceutical acceptable salt thereof (Alpelisib).

In some embodiments, the GSPT1 degrader and the compound of (ii) are administrated together. In some embodiments, the GSPT1 degrader is administrated prior to administration of the compound of (ii). In some embodiments, the compound of (ii) is administrated prior to administration of the GSPT1 degrader.

In some embodiments, the method further comprises administrating a calcium supplement to the patient.

In some embodiments, the cancer has elevated expression of one or more Myc transcription factor biomarkers. In certain embodiments, the one or more Myc transcription factor biomarkers are selected from the group consisting of: L-Myc, N-Myc, c-Myc, EIF4EBP1 and EIF4EBP2. In certain embodiments, the one of more Myc transcription factor biomarkers are selected from the group consisting of: L-Myc and N-Myc.

In some embodiments, the method further comprises a step of determining the expression level of one or more Myc transcription factor biomarkers in a biological sample obtained from the patient. In some embodiments, the step of determining is performed prior to the steps of administration. In some embodiments, the biological sample comprises tumor cells or tumor nucleic acid. In certain embodiments, the tumor nucleic acid is tumor DNA or tumor RNA. In some embodiments, the step of determining comprises acquiring data. In some embodiments, the step of determining comprises obtaining a biological sample and measuring expression or having a biological sample obtained and having expression measured. In some embodiments, the step of determining expression level comprising measuring the copy number a gene encoding a Myc transcription factor biomarker. In certain embodiments, the one or more Myc transcription factor biomarkers are selected from the group consisting of: L-Myc, N-Myc, c-Myc, EIF4EBP1 and EIF4EBP2. In certain embodiments, the one of more Myc transcription factor biomarkers are selected from the group consisting of: L-Myc and N-Myc.

Described herein is a method of treating cancer in a human patient, the method comprising:
identifying a human patient as being in need of treatment for cancer;
testing or having tested, a biological sample obtained from the patient, thereby determining that the patient's cancer exhibits with elevated expression levels of one or more Myc transcription factor biomarkers;
selecting treatment with a GSPT1degrader and a compound of (ii) selected from the group of consisting of a PI3K inhibitor, an Akt inhibitor, and an mTOR inhibitor for the cancer that exhibits elevated expression level of one or more Myc transcription factor biomarkers; and
treating the patient with a GSPT1 degrader and a compound of (ii) selected from the group of a PI3K inhibitor, an Akt inhibitor, and an mTOR inhibitor

Described herein is a method of treating cancer in a human patient, the method comprising:
identifying a human patient having a cancer that is associated with elevated expression levels of one or more Myc transcription factor biomarkers;
selecting treatment with a GSPT1 degrader and a compound of (ii) selected from the group consisting of: a PI3K inhibitor, an Akt inhibitor, and an mTOR inhibitor for the cancer that exhibits elevated expression level of one or more Myc transcription factor biomarkers; and
treating the patient with a GSPT1 degrader and a compound of (ii) selected from the group of a PI3K inhibitor, an Akt inhibitor, and an mTOR inhibitor

Described herein is a method of treating cancer in a human patient, the method comprising
identifying a human patient as being in need of treatment for cancer;
testing or having tested a biological sample obtained from the patient, thereby determining that the patient has elevated expression level of one or more Myc transcription factor biomarkers;
selecting treatment with a GSPT1 degrader and a compound of (ii) selected from the group consisting of: a PI3K inhibitor, an Akt inhibitor, and an mTOR inhibitor for the cancer that exhibits elevated expression level of one or more Myc transcription factor biomarkers; and
treating the patient with a GSPT1 degrader and a compound of (ii) selected from the group of a PI3K inhibitor, an Akt inhibitor, and an TOR inhibitor.

Described herein is a method of treating a patient suffering from cancer comprising administrating: a GSPT1 degrader; and (ii) a compound of (ii) selected from the group consisting of a PI3K inhibitor, an Akt inhibitor, and an mTOR inhibitor, wherein a biological sample obtained from the patient has previously been tested and the testing determined that the cancer has elevated expression level of one or more Myc transcription factor biomarkers.

In some embodiments, the step of determining comprises obtaining a biological sample and measuring expression or having a biological sample obtained and having expression measured. In some embodiments, the biological sample comprises tumor cells or tumor nucleic acid. In certain embodiments, the tumor nucleic acid is tumor DNA or tumor RNA. In some embodiments, the step of determining expression level comprising measuring the copy number a gene encoding a Myc transcription factor biomarker. In some embodiments, the one or more Myc transcription factor biomarkers are selected from the group consisting of: L-Myc, N-Myc, c-Myc, EIF4EBP1 and EIF4EBP2. In some embodiments, the one of more Myc transcription factor biomarkers are selected from the group consisting of: L-Myc and N-Myc. In some embodiments, the expression level of the one or more Myc transcription factor biomarkers is elevated relative to a reference level for that biomarker. In certain embodiments, the expression level is at least 5%, 10%, 15%, 20% or 25% greater than the reference level.

Described herein are certain combination therapies that include the administration of a MGD that degrades GSPT1 and an agent that inhibits the PI3K/AKT/mTOR pathway, e.g., a PI3K inhibitor, an AKT inhibitor or an mTOR inhibitor.

The combination therapies can be used in the treatment of certain cancers and other indications that that can be ameliorated by degradation of GSPT1. In some cases, degradation of GSPT1 may exert a beneficial effect by reducing the level of MYC.

Cancers that may be treated with the combination therapy described herein include solid cancers including, but not limited to, cancers of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, uterus, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

In some embodiments, the combination therapy according to any of the embodiments described herein is used in the treatment of breast cancer, lung cancer (e.g., non-small lung cancer, or small cell lung cancer), lymphoma (e.g., B cell lymphoma) or multiple myeloma.

In some embodiments the combination therapy according to any of the embodiments described herein is used in the treatment of breast cancer.

In some embodiments the combination therapy according to any of the embodiments described herein is used in the treatment of lung cancer, for example, non-small cell lung cancer (e.g., squamous cell lung cancer) and small cell lung cancer.

Some embodiments comprise the use of a compound or a composition according to any of the embodiments described herein for treating neuroendocrine prostate cancer, for example, castration-resistant neuroendocrine prostate cancer (NEPC).

Some embodiments comprise the use of a compound or a composition according to any of the embodiments described herein for treating lung neuroendocrine tumors (Lu-NETs).

Some embodiments comprise the compound or the composition according to any of the embodiments described herein for use in the treatment of acute myelogenous leukemia (AML) and multiple myeloma (MM).

Each of the agents used in the combination therapy may be administered, individually or together, by any suitable means, including oral, topical, transdermal, parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

“Treat” or “treating” a cancer as used herein means to administer a combination therapy described herein to a subject having a cancer, or diagnosed with a cancer, to achieve at least one positive therapeutic effect, such as for example, reduced number of cancer cells, reduced tumor size, reduced rate of cancer cell infiltration, reduced rate of tumor metastasis or reduced rate of tumor growth. Positive therapeutic effects in cancer can be measured in a number of ways, including using the RECIST 1.1 response criteria. Progression-free survival, disease-free survival, complete response, overall survival, and partial response are all measures cancer treatments.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “C₆₋₁₀ aryl” includes both fully aromatic C₆₋₁₀ aryl and partially aromatic C₆₋₁₀ aryl having 6, 7, 8, 9, or 10 ring atoms and includes monocycles and fused bicycles. Examples of fully aromatic C₆₋₁₀ aryl include e.g. phenyl (fully aromatic C₆ aryl), naphthyl (fully aromatic C₁₀ aryl). Examples of partially aromatic C₆₋₁₀ aryl include e.g. indenyl (partially aromatic C₉ aryl), 2,3-dihydroindenyl (partially aromatic C₉ aryl), 1, 2, 3, 4-tetrahydronaphthyl (partially aromatic C₁₀ aryl). In some embodiments for group X¹, C₆₋₁₀ aryl is phenyl. In some embodiments for group X² C₆₋₁₀ aryl is phenyl. The term “—C₁₋₆ alkyl-C₆₋₁₀ aryl” refers to -L²-X¹— or L³-X²— with L², L³ being a C₁₋₆ alkyl group and X¹, X² being a C₆₋₁₀ aryl, and thus refers to a C₆₋₁₀ aryl, which is linked through a C₁₋₆ alkyl group as defined herein to its neighbouring group. The term “—C₁₋₆ alkoxy-C₆₋₁₀ aryl” refers to -L²-X¹— or L³-X²— with L², L³ being a C₁₋₆ alkoxy group and X¹, X² being a C₆₋₁₀ aryl, and thus refers to a C₆₋₁₀ aryl, which is linked through a C₁₋₆ alkoxy group as defined herein to its neighbouring group. The term “—O—C₆₋₁₀ aryl” or “C₆₋₁₀ aryloxy” refers to -L²-X¹— or L3-X²— with L², L³ being —O— and X¹, X² being a C₆₋₁₀ aryl, and thus refers to a C₆₋₁₀ aryl, which is linked through a —O— group to its neighbouring group. The C₆₋₁₀ aryl group may be unsubstituted or substituted with C₁₋₄ alkyl, such as methyl, ethyl, t-butyl, fluorinated C₁₋₄ alkyl, such as —CF₃, —C(CH₃)F₂, C₁₋₄ alkoxy, such as methoxy, ethoxy, fluorinated C₁₋₄ alkoxy, such as —OCF₃, —OCHF₂, CN, —N(Me)₂, halogen, such as F, Cl, or Br, such as F or Cl.

In some embodiments for X¹, a C₆₋₁₀ aryl group refers to a fully aromatic ring system, e.g. phenyl, which is unsubstituted or substituted with C₁₋₄ alkyl, such as methyl, ethyl, t-butyl, fluorinated C₁₋₄ alkyl, such as —CMeF₂, C₁₋₄ alkoxy, such as methoxy, ethoxy, fluorinated C₁₋₄ alkoxy, such as —OCF₃, —OCHF₂, CN, halogen, such as F or Cl.

In some embodiments for X², a C₆₋₁₀ aryl group refers to a fully aromatic ring system, e.g. phenyl, which is unsubstituted or substituted with C₁₋₄ alkyl, such as methyl, ethyl, C₁₋₄ alkoxy, such as methoxy, ethoxy, halogen, such as F, Cl, or Br, such as F or Cl, e.g. F.

The term “5-10 membered heteroaryl” refers to a fully or partially aromatic ring system in form of monocycles or fused bicycles having 5, 6, 7, 8, 9, 10 ring atoms selected from C, N, O, and S, such as C, N, and O, or C, N, and S, with the number of N atoms being e.g. 0, 1, 2 or 3 and the number of O and S atoms each being 0, 1 or 2. In some embodiments a 5-10 membered heteroaryl refers to a fully aromatic ring system having 5, 6, 7, 8, 9, 10, such as 5 or 6, e.g. 6 ring atoms selected from C and N, with the number of N atoms being 1, 2 or 3, such as 1 or 2. In some embodiments a 5-10 membered heteroaryl refers to a fully aromatic ring system having 5, 6, 7, 8, 9, 10, such as 5 or 6, e.g. 5 ring atoms selected from C, N, O, S with the number of N, S and O atoms each being independently 0, 1 or 2. In some embodiments the total number of N, S and O atoms is 2. In some embodiments a 5-10 membered heteroaryl refers to a fully aromatic ring system having 5 ring atoms selected from C, N, S with the number of N and S atoms each being independently 0 or 1. In some embodiments the total number of N and S atoms is 2. In some embodiments a 5-10 membered heteroaryl refers to a fully aromatic ring system having 6 ring atoms selected from C and N, with the number of N atoms being 1 or 2. In other embodiments a 5-10 membered heteroaryl refers to a partially aromatic ring system having 9 or 10 ring atoms selected from C, N and O, with the number of O atoms being 1, 2 or 3, such as 1 or 2, and the number of N atoms being 1 or 2, such as 1. In some embodiments, examples of “5-10 membered heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiophenyl, thiazolyl, thienyl, indolyl, quinazolinyl, oxazolinyl, isoxazolinyl, indazolinyl, isothiazolyl, 1,3-benzodioxolyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, benzodihydropyrane, 1,2,3,4-tetrahydronaphthyl, 2,3-dihydroindenyl and the like. In some embodiments, examples of “5-10 membered heteroaryl” include 5-membered heteroaryl, such as isothiazole, 6-membered heteroaryl, such as pyridinyl, pyrimidinyl, pyridazinyl, pyrazinyl, 9-membered heteroaryl, such as 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, and 10-membered heteroaryl, such as benzodihydropyrane (chromane), dihydropyrano-pyridine. The term “—C₁₋₆ alkyl 5-10 membered heteroaryl” refers to -L²-X¹— or L³-X²— with L², L³ being a C₁₋₆ alkyl group and X¹, X²being a 5-10 membered heteroaryl, and thus refers to a 5-10 membered heteroaryl, which is linked through a C₁₋₆ alkyl group as defined herein to its neighbouring group. The term “—C₁₋₆ alkoxy 5-10 membered heteroaryl” refers to -L²-X¹— or L³-X²— with L², L³ being a C₁₋₆ alkoxy group and X¹, X² being a 5-10 membered heteroaryl, and thus refers to a 5-10 membered heteroaryl, which is linked through a C₁₋₆ alkoxy group as defined herein to its neighbouring group. The term “—O—5-10 membered heteroaryl” refers to -L²-X¹— or L³-X²— with L², L³ being —O— and X¹, X² being a 5-10 membered heteroaryl, and thus refers to a 5-10 membered heteroaryl, which is linked through a —O— group to its neighbouring group. The 5-10 membered heteroaryl group may be unsubstituted or substituted with C₁₋₄ alkyl, such as methyl, ethyl, t-butyl, fluorinated C₁₋₄ alkyl, such as —CF₃, —C(CH₃)F₂, C₁₋₄ alkoxy, such as methoxy, ethoxy, fluorinated C₁₋₄ alkoxy, such as —OCF₃, —OCHF₂, CN, —N(Me)₂, halogen, such as F, Cl, or Br, such as F or Cl. In some embodiments, the 5-10 membered heteroaryl group may be unsubstituted or substituted with C₁₋₄ alkyl, such as methyl, ethyl, t-butyl, fluorinated C₁₋₄ alkyl, such as —CF₃, C₁₋₄ alkoxy, such as methoxy, ethoxy, halogen, such as F or Cl.

In some embodiments for X¹, a 5-10 membered heteroaryl refers to a fully aromatic ring system having 5 ring atoms selected from C, N and S with the number of N and S atoms being independently of each other 0 or 1, e.g. 1 or a fully aromatic ring system having 6 ring atoms selected from C and N, with the number of N atoms being 1 or 2 or a partially aromatic ring system having 9 or 10 ring atoms selected from C, N and O, with the number of O atoms being 1 or 2 and the number of N atoms being 0 or 1. In some embodiments for X¹, a 5-10 membered heteroaryl refers to isothiazole, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine.

In some embodiments for X² a 5-10 membered heteroaryl refers to a fully aromatic ring system having 6 ring atoms selected from C and N, with the number of N atoms being 1 or 2, such as 1. In some embodiments for X² a 5-10 membered heteroaryl refers to pyridinyl.

The term “C₃₋₆ cycloalkyl” refers to a non-aromatic, i.e. saturated or partially unsaturated alkyl ring system, such as monocycles, fused bicycles, bridged bicycles or spirobicycles, containing 3, 4, 5 or 6 carbon atoms. Examples of “C₃₋₈ cycloalkyl” include monocycles, such as cyclopropyl, cyclobutyl, cyclopentyl, cyclohexyl, bridged bicycles, such as bicyclo[1.1.1]pentyl, bicyclo[2.1.1]hexyl, fused bicycles, such as bicyclo[3.1.0]hexyl. The C₃₋₆ cycloalkyl group may be unsubstituted or substituted with C₁₋₄ alkyl, such as methyl, ethyl, t-butyl, fluorinated C₁₋₄ alkyl, such as —CF₃, —CMeF₂, C₁₋₄ alkoxy, such as methoxy, ethoxy, fluorinated C₁₋₄ alkoxy, such as —OCF₃, —OCHF₂, CN, —N(Me)₂, halogen, such as F, Cl, or Br, such as F or Cl. In some embodiments the C₃₋₆ cycloalkyl group may be unsubstituted or substituted by e.g. one or more of C₁₋₄ alkyl, such as methyl and halogen, such as F. In some embodiments for X¹, a C₃₋₆ cycloalkyl refers to cyclopropyl, cyclobutyl.

The term “4-8 membered heterocycloalkyl” refers to a non-aromatic, i.e. saturated or partially unsaturated ring system having 4, 5, 6, 7 or 8 ring atoms (of which at least one is a heteroatom), which ring atoms are selected from C, N, O, and S, such as C, N, and O, the number of N atoms being 0, 1, or 2 and the number of O and S atoms each being 0, 1, or 2. In some embodiments the term “4-8 membered heterocycloalkyl” comprises saturated or partially unsaturated monocycles, fused bicycles, bridged bicycles or spirobicycles. In some embodiments the term “4-8 membered heterocycloalkyl” comprises fully saturated or partially unsaturated monocycles and bridged bicycles. Examples of 4-8 membered heterocycloalkyl groups include azetidinyl, oxetanyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxanyl, 1,3-dithianyl, piperazinyl, thiomorpholinyl, piperidinyl, morpholinyl, azabicyclo[2.2.1]heptan-5-yl, 8-oxa-3-azabicyclo[3.2.1]octan-3-yl and the like. The 4-8 membered heterocycloalkyl group may be unsubstituted or substituted with C₁₋₄ alkyl, such as methyl, ethyl, C₁₋₄ alkoxy, such as methoxy, ethoxy, halogen, such as F, Cl or Br, e.g. F or Cl.

In some embodiments, the 4-8 membered heterocycloalkyl representing group X² is a non-aromatic ring system having 5 or 6 ring atoms of which at least one is a heteroatom selected from N, the number of N atoms being 1 or 2, such as a non-aromatic 5- or 6-membered ring system having 1 or 2 N-atom. Examples include pyrrolidinyl, piperdinyl, morpholinyl, piperazinyl, N-methyl piperazinyl. In some embodiments, the 4-8 membered heterocycloalkyl representing group X² is a non-aromatic ring system having 5 or 6 ring atoms of which one is a N-heteroatom, such as a non-aromatic 5- or 6-membered ring system having 1 N-atom, such as pyrrolidine, piperidine.

The term “halogen” or “hal” as used herein may be fluoro, chloro, bromo or iodo such as fluoro, chloro or bromo, e.g. fluoro or chloro.

The term “C₁₋₄ alkyl” and “C₁₋₆ alkyl” refer to a fully saturated branched or unbranched hydrocarbon moiety having 1, 2, 3 or 4 and 1, 2, 3, 4, 5 or 6 carbon atoms, respectively. Representative examples of alkyl include, but are not limited to, methyl, ethyl, n-propyl, iso-propyl, n-butyl, sec-butyl, iso-butyl, tert-butyl, n-pentyl, iso-pentyl, neopentyl, n-hexyl, iso-hexyl or neohexyl.

The term “C₁₋₆ heteroalkyl” refers to an alkyl as defined with 1, 2, 3, 4, 5 or 6 carbon atoms in which at least one carbon atom is replaced with a heteroatom, such as N, O. It is understood that the heteroatom may further be substituted with one or two C₁₋₆ alkyl. Examples include —(CH₂)₂—O-Me, —(CH₂)₃—O-Me, —(CH₂)₂—O—CH₂Me, —(CH₂)₂—NMe₂, —(CH₂)—NMe₂, —(CH₂)₂-NEt₂, —(CH₂)-NEt₂ and the like.

The term “C₁₋₄alkylamino” refers to a fully saturated branched or unbranched C₁₋₄ alkyl, which is substituted with at least one, such as only one, amino group, alkylamino group or dialkylaminogroup, such as NH₂, HN(C₁₋₄alkyl) or N(C₁₋₄alkyl)₂. Thus, a C₁₋₄alkylamino refers to C₁₋₄alkylamino, C₁₋₄alkyl-(C₁₋₄alkyl)amino, C₁₋₄alkyl-(C₁₋₄dialkyl)amino. Examples include but are not limited to methylaminomethyl, dimethylamonimethyl, aminomethyl, dimethylaminoethyl, aminoethyl, methylaminoethyl, n-propylamino, iso-propylamino, n-butylamino, sec-butylamino, iso-butylamino, tert-butylamino.

The term “C₁₋₄ alkoxy” refers to an unsubstituted or substituted alkyl chain linked to the remainder of the molecule through an oxygen atom, and in particular to methoxy, ethoxy, n-propoxy, iso-propoxy, n-butoxy, iso-butoxy, and t-butoxy.

Based on the definitions given throughout the application the skilled person knows which combinations are synthetically feasible and realistic, e.g. typically combinations of groups leading to some heteroatoms directly linked to each other, e.g. —O—O—, are not contemplated, however synthetically feasible combinations, such as —S—N═ in aisothiazole are contemplated.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a flow diagram of the method used to assess combinations of a GSPT1-directed MGD and a PI3K/AKT/mTOR pathway inhibitor on the killing of cancer cells as described in Examples 1-5.

FIG. 2 shows an overview of compound plate preparation and compound addition for the experiments described in Examples 1-5.

FIG. 3A shows an example of dose response matrix. FIG. 3B shows an example of synergy score matrix. Both FIGS. 3A and 3B apply to Examples 1-5.

FIG. 4A shows mean Loewe synergy scores for combinations of compound 1-1 and various PI3K/AKT/mTOR pathway inhibitors in various breast cancer cell lines. FIG. 4B shows mean Bliss synergy scores for combinations of compound 1-1 and various PI3K/AKT/mTOR pathway inhibitors in various breast cancer cell lines. In FIGS. 4A and 4B, Compound 1-1 was tested from 1 uM to 4.1 nM. PI3K/AKT/mTOR pathway inhibitors were tested from 10 uM to 41 nM.

FIG. 5A shows mean Loewe synergy scores for combinations of compound 1-2, compound 1-3, compound 1-4, compound 1-5, or compound 1-6 and certain PI3K/AKT/mTOR pathway inhibitors in breast cancer cell lines. FIG. 5B shows mean Bliss synergy scores for combinations of compound 1-2, compound 1-3, compound 1-4, compound 1-5, and compound 1-6 and various PI3K/AKT/mTOR pathway inhibitors in breast cancer cell lines. In FIGS. 5A and 5B, compounds 1-2 through 1-6 were tested from 100 nM to 0.41 nM. PI3K/AKT/mTOR pathway inhibitors were tested from 10 uM to 41 nM.

FIG. 6A shows mean Loewe synergy scores for combinations of compound 1-1 and various PI3K/AKT/mTOR pathway inhibitors in certain lung cancer cell lines. FIG. 6B shows mean Bliss scores for combinations of compound 1-1 with various PI3K/AKT/mTOR pathway inhibitors in lung cancer cell lines. In FIGS. 6A and 6B, compound 1-1 was tested from 500 nM to 1.1 nM. PI3K/AKT/mTOR pathway inhibitors were tested from 10 uM to 21 nM, except for Everolimus, which was tested from 1 uM to 2.1 nM.

FIG. 7A shows mean Loewe synergy scores for combinations of compound 1-6 and various PI3K/AKT/mTOR pathway inhibitors in breast cancer cell lines. FIG. 7B shows mean Bliss synergy scores for combinations of compound 1-6 and various PI3K/AKT/mTOR pathway inhibitors in breast cancer cell lines. In FIGS. 7A and 7B, Compound 1-6 was tested from 100 nM to 0.41 nM. PI3K/AKT/mTOR pathway inhibitors were tested from 10 uM to 41 nM.

FIG. 8A shows mean Loewe synergy scores for combinations of compound 1-6 and various PI3K/AKT/mTOR pathway inhibitors in lung cancer cell lines. FIG. 8B shows mean Bliss scores for combinations of compound 1-6 and various PI3K/AKT/mTOR pathway inhibitors in lung cancer cell lines. In FIGS. 8A and 8B, compound 1-6 was tested from 100 nM to 0.21 nM. PI3K/AKT/mTOR pathway inhibitors were tested from 10 uM to 21 nM, except for Everolimus, which was tested from 1 uM to 2.1 nM.

FIG. 9A-9L show % inhibition and Loewe synergy scores for combination of compound 1-1 and everolimus in six non-small cell lung cancer cell lines: NCIH1770, NCIH2106, ABC1, NCIH441, EBC1, NCI-H2023.

FIG. 10A-10J show % inhibition and Loewe synergy scores for compound 1-1 and everolimus in five small cell lung cancer cell lines: NCIH1836, NCIH1876, NCIH209, NCIH526, NCIH69.

FIG. 11A-11J show % inhibition and Loewe synergy scores for compound 1-1 and everolimus in four lymphoma cell lines: DOHH2, DB, WSUDL, RAJI, SUDHL6.

FIG. 12 shows the results of in vivo combination of compound 1-1 and the mTOR inhibitor everolimus in tumor-bearing mice. Immunodeficient BALB/c nude mice bearing subcutaneous MCF7 xenografts were treated with compound 1-1, everolimus, a combination of both agents, or vehicle control at the indicated doses once daily. Treatments started when implanted tumor cells had formed tumors of approximately 200 mm³ and lasted 28 consecutive days. Each group consisted of 4 mice. Left graph: Tumor volumes over time. Right graph: Body weights over time. Statistics on Δ tumor volumes and A body weights at the end of the study (day 28) were performed with one-way ANOVA and a post hoc Tukey's test for pair-wise comparisons of groups. P-values indicating significant differences between groups are symbolized by asterisks as follows: * p<0.05, ** p<0.01, *** p<0.001, **** p<0.0001). Asterisks on a treatment group mark difference to vehicle control, differences between treatment groups are shown with lines linking the respective data points in the graph. The same statistical tests were also performed with the tumor volume data on day 21, revealing that—unlike on day 28—both the compound 1-1 single agent group and the everolimus single agent group were significantly different from the combination group (p=0.008 and p=0.01, respectively). No significant changes in body weight were detected. (mpk means milligram drug per kilogram bodyweight; BW means body weight.) FIG. 13 shows the results of in vivo combination of compound 1-1 and the PI3K inhibitor alpelisib in tumor-bearing mice. Immunodeficient BALB/c nude mice bearing subcutaneous MCF7 xenografts were treated with compound 1-1, alpelisib, a combination of both agents, or vehicle control at the indicated doses once daily. Treatments started when implanted tumor cells had formed tumors of approximately 200 mm³ and lasted 28 consecutive days. Each group consisted of 4 mice. Left graph: Tumor volumes over time. Right graph: Body weights over time. Statistics on Δ tumor volumes and A body weights at the end of the study (day 28) were performed with one-way ANOVA and a post hoc Tukey's test for pair-wise comparisons of groups. P-values indicating significant differences between groups are symbolized by asterisks as follows: * p<0.05, ** p<0.01, *** p<0.001, **** p<0.0001). Asterisks on a treatment group mark difference to vehicle control, differences between treatment groups are shown with lines linking the respective data points in the graph. No significant changes in body weight were detected. (mpk means milligram drug per kilogram bodyweight; BW means body weight.)

FIG. 14 shows the results of in vivo combination of compound 1-1 and the mTOR inhibitor everolimus in tumor-bearing mice. Immunodeficient SCID Beige mice bearing subcutaneous CAL51 xenografts were treated with compound 1-1, everolimus, a combination of both agents, or vehicle control at the indicated doses once daily. Treatments started when implanted tumor cells had formed tumors of approximately 200 mm³ and lasted 21 consecutive days. Each group consisted of 4 mice. Left graph: Tumor volumes over time. Right graph: Body weights over time. Statistics on A tumor volumes and A body weights at the end of the study (day 21) were performed with one-way ANOVA and a post hoc Tukey's test for pair-wise comparisons of groups. P-values indicating significant differences between groups are symbolized by asterisks as follows: * p<0.05, ** p<0.01, *** p<0.001, **** p<0.0001). Asterisks on a treatment group mark difference to vehicle control, differences between treatment groups are shown with lines linking the respective data points in the graph. No significant changes in body weight were detected. (mpk means milligram drug per kilogram bodyweight; BW means body weight.)

FIG. 15 shows the results of in vivo combination of compound 1-1 and the PI3K inhibitor alpelisib in tumor-bearing mice. Immunodeficient SCID Beige mice bearing subcutaneous CAL51 xenografts were treated with compound 1-1, alpelisib, a combination of both agents, or vehicle control at the indicated doses once daily. Treatments started when implanted tumor cells had formed tumors of approximately 200 mm³ and lasted 21 consecutive days. Each group consisted of 4 mice. Left graph: Tumor volumes over time. Right graph: Body weights over time. Statistics on Δtumor volumes and Δ body weights at the end of the study (day 21) were performed with one-way ANOVA and a post hoc Tukey's test for pair-wise comparisons of groups. P-values indicating significant differences between groups are symbolized by asterisks as follows: * p<0.05, ** p<0.01, *** p<0.001, **** p<0.0001). Asterisks on a treatment group mark difference to vehicle control, differences between treatment groups are shown with lines linking the respective data points in the graph. No significant changes in body weight were detected. (mpk means milligram drug per kilogram bodyweight; BW means body weight).

FIG. 16A-FIG. 16H show the results of in vivo combination of compound 1-1 and the mTOR inhibitor everolimus in tumor-bearing mice. Immunodeficient BALB/c nude mice bearing subcutaneous NCI-H1155 xenografts were treated with compound 1-1, everolimus, a combination of both agents, or vehicle control at a total of 15 different dose regimens as indicated, where “combination” means a combination of the two single agent regimens in the respective graph. Everolimus was generally dosed once daily. Compound 1-1 was either dosed once daily (qd) or in an intermittent regimen of 5 consecutive daily doses followed by 9 days of drug holiday (5 days on/9 days off). Treatments started when implanted tumor cells had formed tumors of approximately 200 mm3 and lasted until tumors had exceeded a volume of 2000 mm3 up to a maximal duration of 43 days. (Most groups were terminated earlier as described in Example 14). Tumor volumes were measured twice per week. Each group consisted of 5 mice. Given the rapid and heterogenous growth of NCI-H1155 tumors that quickly leads to unequal group sizes due to exceeding the maximal tumor volume, anti-tumor efficacy is displayed as Kaplan-Meier (Probability of Survival) curves, where survival was defined as tumor volume ≤800 mm3. Therefore, each drop of the curve by 20% represents one mouse in which tumor volume reached or exceeded 800 mm3. Log-rank (Mantel-Cox) tests were performed comparing the combination treatment group to the more efficacious of the two respective single agent groups. Statistically significant superiority of the combination regimen is symbolized by asterisks as follows: * p<0.05, ** p<0.01. For better readability, the efficacy data is displayed in 8 different panels (FIG. 16A-H) where each panels shows the vehicle group, one single agent regimen of compound 1-1 and everolimus, respectively, and the combination of those two single agent regimens. Therefore, vehicle as well as single agent data is repeated across multiple panels of FIG. 16. (mpk means milligram drug per kilogram bodyweight).

FIG. 17A-FIG. 17H shows the results of in vivo combination of compound 1-1 and the mTOR inhibitor everolimus in tumor-bearing mice. Immunodeficient BALB/c nude mice bearing subcutaneous NCI-H1770 xenografts were treated with compound 1-1, everolimus, a combination of both agents, or vehicle control at a total of 15 different dose regimens as indicated, where “combination” means a combination of the two single agent regimens in the respective graph. Everolimus was generally dosed once daily. Compound 1-1 was either dosed once daily (qd) or in an intermittent regimen of 5 consecutive daily doses followed by 9 days of drug holiday (5 days on/9 days off). Treatments started when implanted tumor cells had formed tumors of approximately 160 mm3 and lasted until tumors had exceeded a volume of 2000 mm3 up to a maximal duration of 49 days. A subset of groups was terminated earlier as described in Example 15. Tumor volumes were measured twice per week. Each group consisted of 5 mice. Given the rapid and heterogenous growth of NCI-H1770 tumors that quickly leads to unequal group sizes due to exceeding the maximal tumor volume, anti-tumor efficacy is displayed as Kaplan-Meier (Probability of Survival) curves, where survival was defined as tumor volume ≤800 mm3. Therefore, each drop of the curve by 20% represents one mouse in which tumor volume reached or exceeded 800 mm3. Log-rank (Mantel-Cox) tests were performed comparing the combination treatment group to the more efficacious of the two respective single agent groups. Statistically significant superiority of the combination regimen is symbolized by asterisks as follows: * p<0.05, ** p<0.01. For better readability, the efficacy data is displayed in 8 different panels (FIG. 17A-H) where each panels shows the vehicle group, one single agent regimen of compound 1-1 and everolimus, respectively, and the combination of those two single agent regimens. Therefore, vehicle as well as single agent data is repeated across multiple panels of FIG. 17. (mpk means milligram drug per kilogram bodyweight).

FIG. 18A-FIG. 18F shows the results of in vivo combination of compound 1-1 and the mTOR inhibitor everolimus in mice bearing patient-derived xenografts (PDX). Six different lung cancer PDX models named LU5188, LU5247, LU5236, LU1508, LU5215, and LU5137 were tested, tumor volume data are presented in 6 different panels labeled FIG. 18A-H. Subcutaneous tumors were grown in either immunodeficient BALB/c nude (LU1508) or immunodeficient NOD/SCID (all others) mice. Groups of 6 mice were treated with compound 1-1, everolimus, a combination of both agents, or vehicle control. Everolimus was generally dosed at 5 mg/kg (mpk) once daily (qd). Compound 1-1 was dosed in two different regimens that were chosen individually for each model and entailed either daily (qd) or intermittent dosing (5 days on/9 days off as in FIG. 16/17). Both compound 1-1 regimens were also combined with the constant everolimus dosing regimen. For better readability, the data for each model was split into an upper and lower graph where the respective lower compound 1-1 dose and its combination are in the upper graph and the higher dose in the lower graph. Vehicle and everolimus curves are thus repeated in those two graphs. Treatment groups were terminated individually based on observed anti-tumor efficacy when either a combination benefit became apparent (e.g., FIG. 18A) or when a combination benefit became unlikely (e.g., FIG. 18B upper graph). Vertical dotted lines indicate the last test article administration, after which tumor volumes were observed without further treatment. In graphs without a vertical dotted line, test article administration was continued until the last displayed data points.

### DETAILED DESCRIPTION

Described herein is a method of treating cancer including PIK3CA mutant tumors using a combination of a GSPT1 MGD and a PI3K/AKT/mTOR pathway inhibitor.

### GSPT1 MOLECULAR GLUE DEGRADER

Molecular glue degraders (MGDs) targeted to GSPT1 (GSPT1-directed MGDs) are described, for example, in: PCT/WO2021/069705, PCT/EP2022/050699 and PCT/US2022/24796 all of which are hereby incorporated by reference in its entirety. Other GSPT1-directed MGDs are described in WO 2021/069705, WO 2022/066835, and WO 2019/241271, all of which are hereby incorporated by reference in its entirety.

In some cases, the GSPT1-directed MGD is a compound shown in Table 1, or a pharmaceutically acceptable salt thereof, enantiomer thereof, or a stereoisomer thereof.

TABLE 1
GSPT1 molecular glue degraders

| Compound | | |
|---|---|---|
| No. | Structure | Compound IUPAC Name |
| 1-1 | 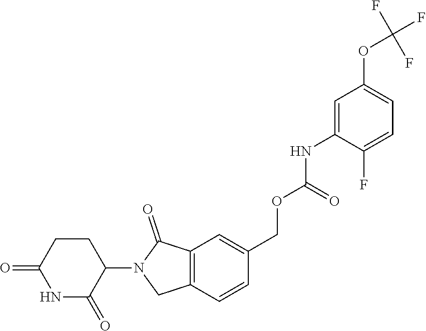 [SMILES: O=C1CCC(N2Cc3ccc(COC(=O)Nc4cc(OC(F)(F)F)ccc4F)cc3C2=O)C(=O)N1] | [2-(2,6-dioxopiperidin-3-yl)-3-oxo-2,3- dihydro-1H-isoindol-5-yl)methyl N-[2- fluoro-5- (trifluoromethoxy)phenyl]carbamate |
| 1-2 | 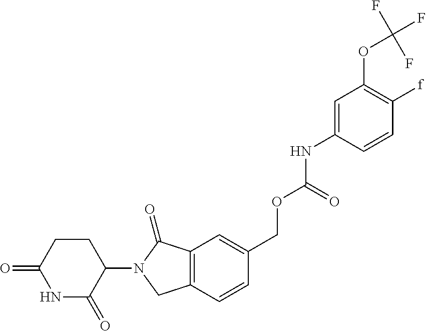 | [2-(2,6-dioxopiperidin-3-yl)-3-oxo-2,3- dihydro-1H-isoindol-5-yl]methyl N-[4- fluoro-3- (trifluoromethoxy)phenyl]carbamate |
| 1-3 | 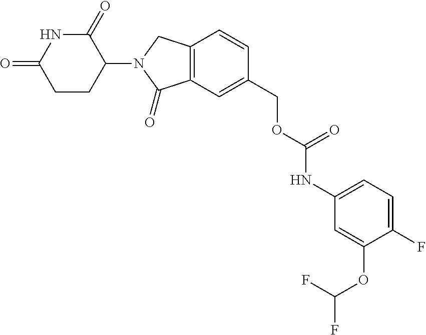 [SMILES: O=C1CCC(N2Cc3ccc(COC(=O)Nc4ccc(F)c(OC(F)F)c4)cc3C2=O)C(=O)N1] | [2-(2,6-dioxopiperidin-3-yl)-3-oxo-2,3- dihydro-1H-isoindol-5-yl]methyl N-[3- (difluoromethoxy)-4- fluorophenyl]carbamate |
| 1-4 | 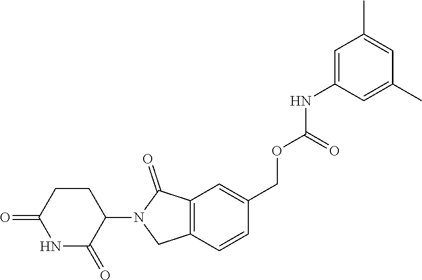 [SMILES: Cc1cc(C)cc(NC(=O)OCc2ccc3c(c2)C(=O)N(C2CCC(=O)NC2=O)C3)c1] | [2-(2,6-dioxopiperidin-3-yl)-3-oxo-2,3- dihydro-1H-isoindol-5-yl]methyl N-(3,5- dimethylphenyl)carbamate |
| 1-5 | 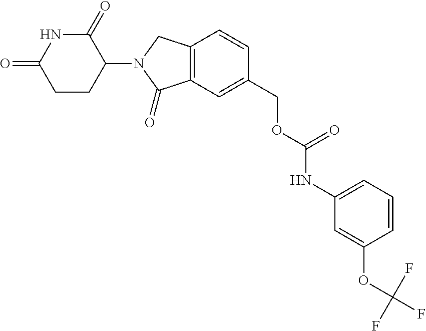 [SMILES: O=C1CCC(N2Cc3ccc(COC(=O)Nc4cccc(OC(F)(F)F)c4)cc3C2=O)C(=O)N1] | [2-(2,6-dioxopiperidin-3-yl)-3-oxo-2,3- dihydro-1H-isoindol-5-yl]methyl N-[3- (trifluoromethoxy)phenyl]carbamate |
| 1-6 | 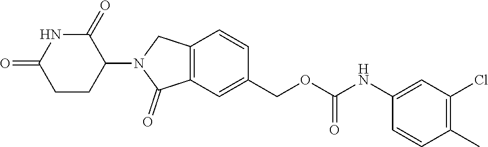 [SMILES: Cc1ccc(NC(=O)OCc2ccc3c(c2)C(=O)N(C2CCC(=O)NC2=O)C3)cc1Cl] | [2-(2,6-dioxopiperidin-3-yl)-3-oxo-2,3- dihydro-1H-isoindol-5-yl]methyl N-(3- chloro-4-methylphenyl)carbamate |
| 1-7 | 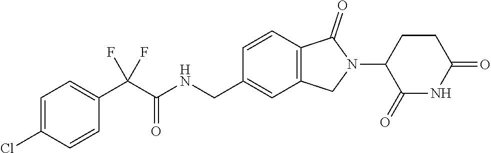 [SMILES: O=C1CCC(N2Cc3cc(CNC(=O)C(F)(F)c4ccc(Cl)cc4)ccc3C2=O)C(=O)N1] | 2-(4-chlorophenyl)-N-((2-(2,6- dioxopiperidin-3-yl)-1-oxoisoindolin-5- yl)methyl)-2,2-difluoroacetamide |
| 1-8 | 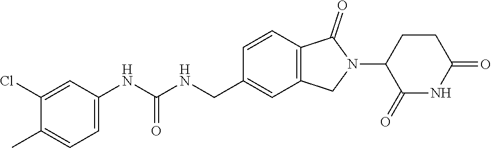 [SMILES: Cc1ccc(NC(=O)NCc2ccc3c(c2)CN(C2CCC(=O)NC2=O)C3=O)cc1Cl] | N-(3-chloro-4-methylphenyl)-N′-[[2- (2,6-dioxo-3-piperidinyl)-2,3-dihydro-1- oxo-1H-isoindol-5-yl]methyl]-urea |

### PT3K/AKT/mTOR INHIBITORS

In some cases, the PI3K/AKT/mTOR inhibitor is a compound shown in Table 2, or a pharmaceutically acceptable salt thereof, enantiomer thereof, or a stereoisomer thereof.

TABLE 2
PI3K, AKT, and mTOR pathway inhibitors

| Cpd | | Compound IUPAC | PubChem | |
|---|---|---|---|---|
| Name | Structure | Name | CID | Activity |
| Alpelisib | 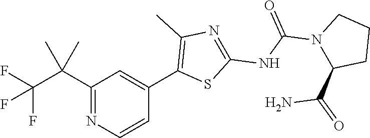 [SMILES: Cc1nc(NC(=O)N2CCC[C@H]2C(N)=O)sc1-c1ccnc(C(C)(C)C(F)(F)F)c1] | (2S)-1-N-[4-methyl-5- [2-(1,1,1-trifluoro-2- methylpropan-2- yl)pyridin-4-yl]-1,3- thiazol-2- yl]pyrrolidine-1,2- dicarboxamide | 56649450 | PI3KCA Inhibitor |
| GNE-477 | 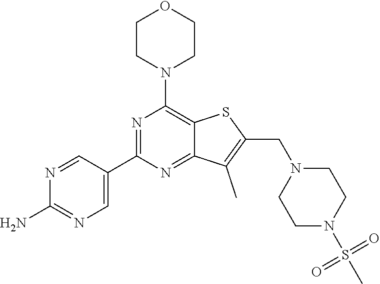 [SMILES: Cc1c(CN2CCN(S(C)(=O)=O)CC2)sc2c(N3CCOCC3)nc(-c3cnc(N)nc3)nc12] | 5-[7-methyl-6-[(4- methylsulfonylpiperazin- 1-yl)methyl]-4- morpholin-4- ylthieno[3,2- d]pyrimidin-2- yl]pyrimidin-2-amine | 25207689 | Pan- PI3K/ mTOR Inhibitor |
| HS-173 | 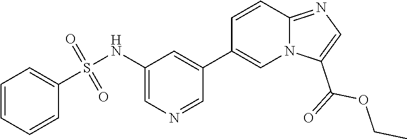 [SMILES: CCOC(=O)c1cnc2ccc(-c3cncc(NS(=O)(=O)c4ccccc4)c3)cn12] | ethyl 6-[5- (benzenesulfonamido) pyridin-3- yl]imidazo[1,2- a]pyridine-3- carboxylate | 52936849 | PI3KCA Inhibitor |
| Serabelisib | 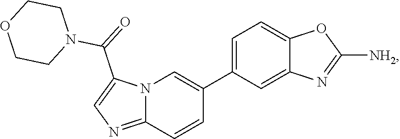 [SMILES: Nc1nc2cc(-c3ccc4ncc(C(=O)N5CCOCC5)n4c3)ccc2o1] | [6-(2-amino-1,3- benzoxazol-5- yl)imidazo[1,2- a]pyridin-3-yl]- morpholin-4- ylmethanone | 70798655 | PI3KCA Inhibitor |
| Capivasertib | 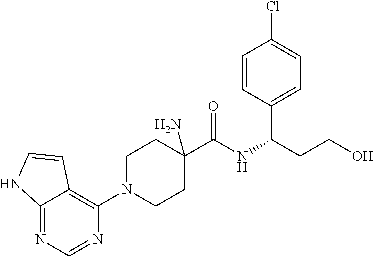 [SMILES: NC1(C(=O)N[C@@H](CCO)c2ccc(Cl)cc2)CCN(c2ncnc3[nH]ccc23)CC1] | 4-amino-N-[(1S)-1-(4- chlorophenyl)-3- hydroxypropyl]-1- (7H-pyrrolo[2,3- d]pyrimidin-4- yl)piperidine-4- carboxamide | 25227436 | AKT Inhibitor |
| M2698 | 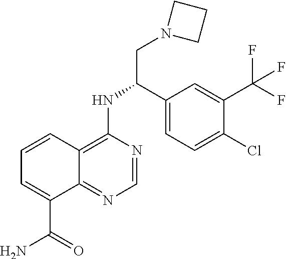 [SMILES: NC(=O)c1cccc2c(N[C@H](CN3CCC3)c3ccc(Cl)c(C(F)(F)F)c3)ncnc12] | 4-[[(1S)-2-(azetidin-1- yl)-1-[4-chloro-3- (trifluoromethyl)phenyl] ethyl]amino]quinazoline- 8-carboxamide | 89808643 | AKT Inhibitor |
| Everolimus | 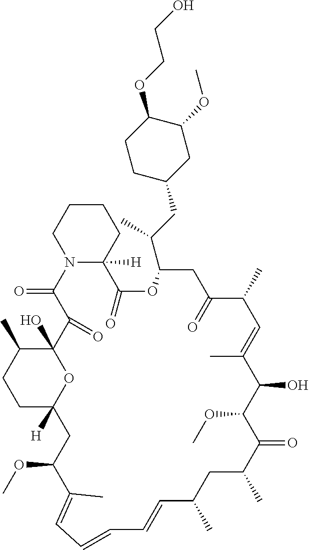 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C] | (1R,9S,12S,15R,16E,18R, 19R,21R,23S,24E,26E, 28E,30S,32S,35R)- 1,18-dihydroxy-12- [(2R)-1-[(1S,3R,4R)-4- (2-hydroxyethoxy)-3- methoxycyclohexyl]pro- pan-2-yl]-19,30- dimethoxy- 15,17,21,23,29,35- hexamethyl-11,36- dioxa-4- azatricyclo[30.3.1.0^{4,9}] hexatriaconta- 16,24,26,28-tetraene- 2,3,10,14,20-pentone | 6442177 | mTOR Inhibitor |
| KU- 0063794 | 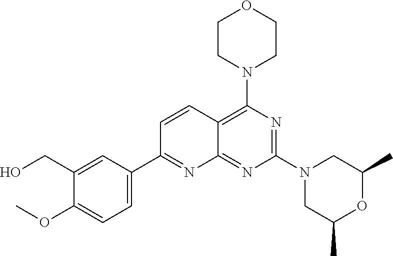 [SMILES: COc1ccc(-c2ccc3c(N4CCOCC4)nc(N4C[C@@H](C)O[C@@H](C)C4)nc3n2)cc1CO] | [5-[2-[(2R,6S)-2,6- dimethylmorpholin-4- yl]-4-morpholin-4- ylpyrido[2,3- d]pyrimidin-7-yl]-2- methoxyphenyl]metha- nol | 16736978 | mTOR Inhibitor |
| buparlisib | 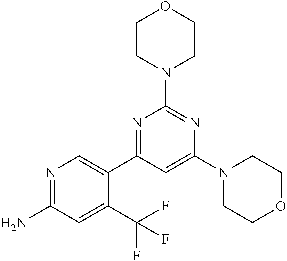 [SMILES: Nc1cc(C(F)(F)F)c(-c2cc(N3CCOCC3)nc(N3CCOCC3)n2)cn1] | 5-(2,6-dimorpholin-4- ylpyrimidin-4-yl)-4- (trifluoromethyl)pyridin- 2-amine | 16654980 | Pan-PI3K |
| idelalisib | 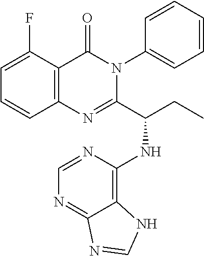 [SMILES: CC[C@H](Nc1ncnc2nc[nH]c12)c1nc2cccc(F)c2c(=O)n1-c1ccccc1] | 5-fluoro-3-phenyl-2- [(1S)-1-(7H-purin-6- ylamino)propyl]quinazo- lin-4-one | 11625818 | PI3K delta |

### Protocol for Assessing Cancer Cell Killing

A study was undertaken to determine whether the combination of a GSPT1-directed MGD and an inhibitor of the PI3K/AKT/mTOR pathway could be effective in killing cancer cells and whether certain combination could elicit a greater than additive effect on cell killing.

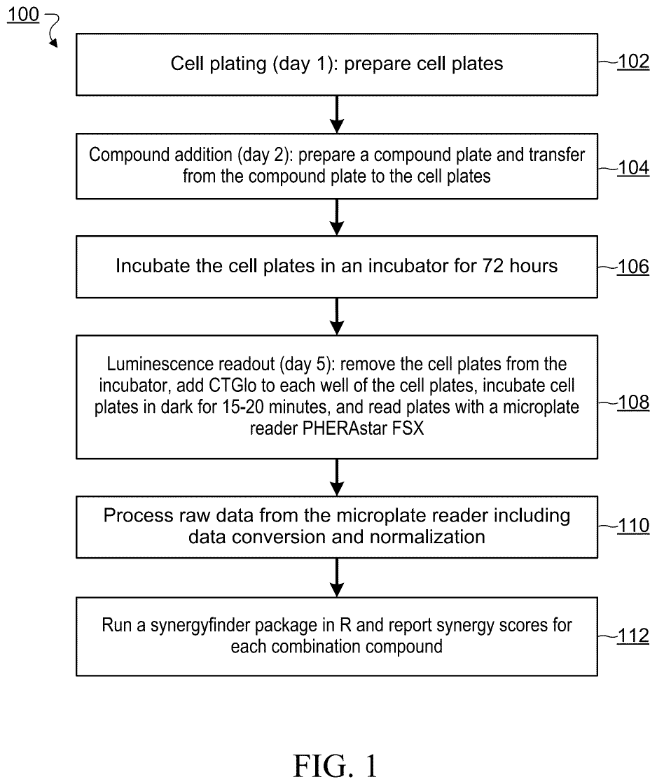

FIG. 1 is a flow diagram of the method used to assess cancer cell killing by a combination of a GSPT1-directed MGD and an inhibitor of the PI3K/AKT/mTOR pathway. Certain of the materials and equipment used in the studies are presented in Table 3 and the cell lines used are presented in Table 4.

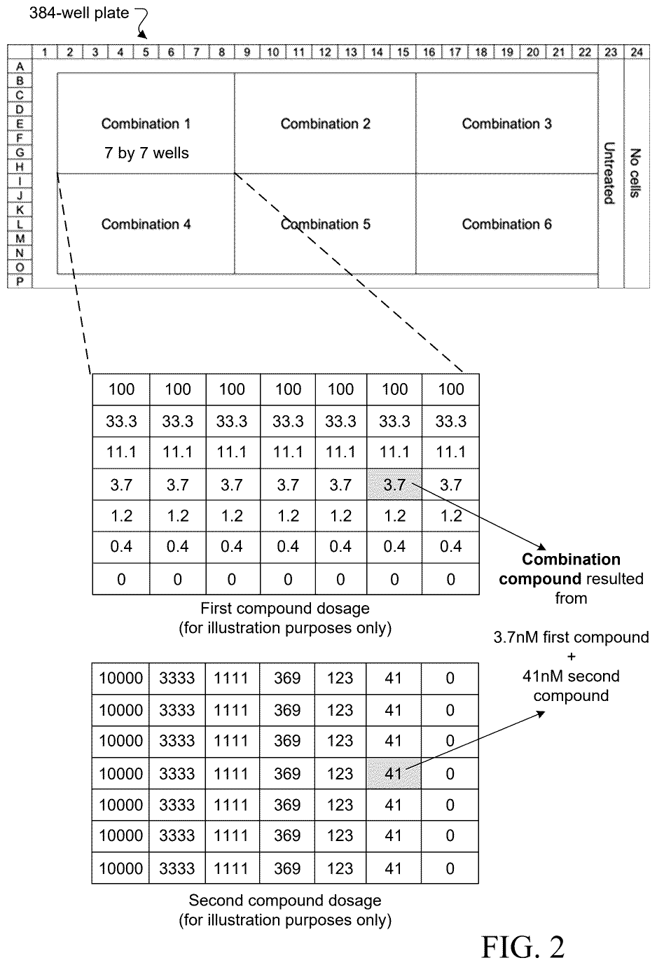

All assays were run in triplicate. Each 384-well plate accommodated six 7×7 dose response matrices (FIG. 2). All compound handling was performed using an Echo 650 Series Acoustic Liquid Handler, which enables fully automated, precise, contact-free acoustic transfer in volumes as low as 2.5 nL.

TABLE 3
Materials and equipment

| Material | Equipment |
|---|---|
| RPMI 1640 Medium, no phenol red | ThermoFisher Scientific, 11835030 |
| DMEM, high glucose, HEPES, no phenol red | ThermoFisher Scientific, 21063029 |
| Fetal Bovine Serum, Tetracycline Negative, Premium, | Corning, 35-075-CV |
| United States Origin | |
| Penicillin-Streptomycin, 10,000 U/mL | ThermoFisher Scientific, 15140-122 |
| 500 mL Vacuum Filter/Storage Bottle System, 0.22 μm Pore | Corning, 431097 |
| 33.2 cm² PES Membrane, Sterile | |
| PBS, pH 7.4 | ThermoFisher Scientific, 10010023 |
| Ethanol, 70% | VWR, 97065-058 |
| Trypsin-EDTA, 0.25%, phenol red | Thermo Fisher Scientific, 25200056 |
| Tissue Culture Flasks, Sterile, Corning | VWR, 353112 |
| Cellometer Auto T4 Bright Field Cell Counter | Nexcelom Bioscience |
| Trypan Blue Solution, 0.4% | ThermoFisher, 15250061 |
| Cell Strainers, DNase/RNase Free, Non-Pyrogenic, Sterile | VWR, 76327-098 |
| 384-well Flat Clear Bottom Black Polystyrene TC-treated | Corning, 3764BC |
| Microplates, Low Flange | |
| MultiFlo FX Multi-Mode Dispenser or Multidrop Combi | BioTek or Thermo Fisher Scientific, |
| Reagent Dispenser | 5840300 |
| Small tube, metal tip dispensing cassette (0.5-50 μL | Those compatible with liquid |
| dispensing range) | handler of choice |
| Echo 650 Series Acoustic Liquid Handlers | Beckman Coulter |
| Echo Qualified 384-well COC Source Microplate, Low Dead | Labcyte, LP-0200 |
| Volume | |
| Echo Qualified 384-Well Polypropylene Microplate 2.0 | Labcyte, PP-0200 |
| CellTiter-Glo 2.0 Cell Viability Assay | Promega, G9243 |
| PHERAstar FSX | BMG Labtech |

TABLE 4
Cell lines

| Cell line | Description |
|---|---|
| CAL51 | Triple negative breast cancer cell line (PIK3CA E542K mutation) |
| MCF7 | Estrogen receptor positive (ER+) breast cancer cell line (PIK3CA E545K |
| | mutation) |
| MDA-MB- | Triple negative breast cancer cell line (PIK3CA WT) |
| 157 | |
| MDA-MB- | Triple negative breast cancer cell line (PIK3CA WT) |
| 231 | |
| CAL51 | Engineered cell line, where GSPT1 molecular degraders can no longer effectively |
| mutant | recruit Cereblon to GSPT1 |
| ABC-1 | Non-small cell lung cancer cell line (N-MYC high expression) |
| NCI-H1155 | Non-small cell lung cancer cell line (N-MYC high expression, neuroendocrine) |
| NCI-H2023 | Non-small cell lung cancer cell line (N-MYC low expression) |
| EBC-1 | Non-small cell lung cancer cell line (N-MYC low expression) |

The following protocol was used for cell plating:
1. Warm media to room temperature or 37° C.
2. Prepare complete phenol-free media by adding 10% FBS+1% Pen-Strep to RPMI 1640 Medium or DMEM.
3. Aspirate media from T175 tissue culture flask and rinse with 10 ml PBS.
4. Aspirate PBS and add 2 mL 0.25% trypsin. Incubate the flask at 37° C. for 5 min or until cells are detached.
5. Add 10 mL complete media to the flask to neutralize the trypsin, then collect cells in a 50 mL conical tube.
6. Centrifuge cells at 300 g for 5 min. Aspirate media and suspend cell pellet in fresh media. Use cell strainer to strain cells into a new 50 mL conical tube.
7. Count cells using the Cellometer Auto T4 Bright Field Cell Counter or a comparable cell counter capable of distinguishing dead and live cells.
   a. Mix 100 uL cell suspension with 100 uL trypan blue (1:1) and pipette 20 μL of the mixture into each of 2 counting chambers on a slide. Insert slide.
   b. Adjust focus and record the live cell count. At least 2 independent measurements should be taken. If the measurements vary significantly, 2 additional measurements should be taken.
   c. Cell viability should be greater than 90%. If cell viability is below 90%, do not proceed with plating cells for the experiment.
8. Dilute cell suspension to the desired concentration (20000 cells/mL) using complete phenol-free media. Prepare 10 mL cell suspension per plate+an extra 10 mL for priming the liquid handler.
9. Dispense cells into 384-well plates using the liquid handler at 25 uL/well (500 cells/well). 3 plates should be prepared per compound plate to be treated.
10. Cells should be dispensed into columns 1-23 only. Media only (no cells) should be dispensed into column 24.
11. Allow plates to rest at RT for 30 minutes before transferring to the incubator. Incubate cells overnight at 37° C., 95% humidity, and 5% CO2.

Compound plates were prepared as follows using an Echo 650 Series Acoustic Liquid Handler.
1. A compound plate containing 7-point dose responses of each compound to be treated is first prepared from stock compounds.
2. The cell plates are treated with compounds according to the following maps, where the first compound is added across all six 7 by 7 dose response matrices, and its concentration varies by row (FIG. 2; top matrix), whereas the second compound varies for each dose response matrix, and its concentration varies by column (FIG. 2; bottom matrix). Each cell plate receives one Compound A and six different Compound Bs.
3. To prepare a compound plate, 384-well PP plate with stock compounds (highest concentration) is prepared. Compounds are diluted 1:1000 in the cell plates, e.g. 10 mM on compound plate will be 10 uM on cell plate. A 384-well LDV plate with pre-determined volumes of DMSO is prepared. Echo is used to dilute stock compounds from PP plate into LDV plate to prepare 7-point dose responses.

Compounds were added to the cells and cell viability was measured after 72 hour incubation using CellTiter-Glo 2.0 Cell Viability Assay (Promega):
1. For a compound addition, Echo is used to transfer 25 nL from the compound plate wells to the cell plates according to the plate maps shown in FIG. 2. Cell plates are treated in triplicate. Each well of the cell plate has 25 uL media (1:1000 dilution). Incubate cells post-treatment at 37° C., 95% humidity, and 5% CO2 for 72 hrs (step 106).
2. The cell plates are removed from incubator and left in hood for 10-20 minutes to equilibrate to RT. CTGlo is diluted with PBS with 1:1 ratio. 5 μL of CTGlo is added to each well of the cell plates using liquid handler. The cell plates are incubated at RT for 15-20 minutes. A microplate reader PHERAstar FSX is used to read plates at RT using the “LUM plus” optic module with the following setting: gain of 4095, measurement interval time of 0.2s, and 384-well aperture spoon.

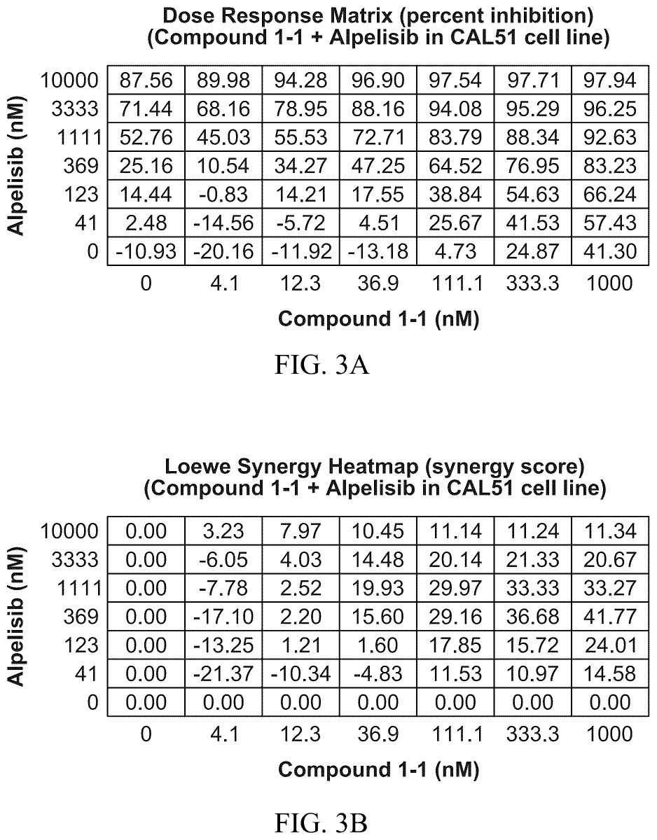

Raw data from the PHERAstar was first processed and converted into percent inhibition values using R. FIG. 3A shows an example dose response matrix including percent inhibition for each combination resulted from the first compound with a first dose and the second compound with a second dose. All values were normalized according to the negative controls (untreated cells, 0% response) and the positive controls (no cells, 100% response) on a plate-by-plate basis. The % inhibition response values are converted to a format adequate for quantifying synergy.

The synergyfinder package in R (Zheng et al. bioRxiv 2021, 10.1101/2021.06.01.446564) was used to perform synergy scoring. FIG. 3B shows an example synergy heatmap including a synergy score for each combination resulted from the first compound at a given concentration and the second compound at a given concentration. For each combination of the first and the second compound, synergy scores were computed using the Loewe or Bliss reference models. These two models are based on different definitions of the expected effect of a given drug combination under the assumption of no drug interaction, which ultimately leads to slightly different synergy scores. In brief, the Loewe additivity model defines the expected effect as if a compound was combined with itself. The Bliss independence model assumes a stochastic process in which two compounds exert effects independently, and the expected combination effect can be estimated based on the probability of independent events (e.g., Bliss score=effect of a first compound+effect of a second compound−effect of a first compound * effect of a second compound). Synergy scores quantify the deviation from the expected effect according to the respective reference model. For each combination, the mean synergy score across the entire dose response matrix (e.g., matrix shown in FIG. 3B) was computed as a convenient way to summarize the overall effect.

### EXAMPLES

The following examples are included for illustrative purposes only and are not intended to limit the scope of the invention.

Abbreviations:

| | |
|---|---|
| SCID | severe combined immunodeficiency |
| GSPT1 | G to S phase transition 1 |
| mTOR | mechanistic target of rapamycin kinase |
| TV | tumor volume |
| BW | body weight |
| Δ TV | differences between tumor volumes in the same animal on day 0 and at the |
| | time point of interest (e.g. end of the study) |
| Δ BW | differences between body weight of the same animal on day 0 and at the |
| | time point of interest (e.g. end of the study) |
| PI 3-kinase | collective term for a family of phosphoinositide 3-kinases |
| PIK3CA | phosphatidylinositol-4,5-bisphosphate 3-kinase catalytic subunit alpha, an |
| | oncogene frequently mutated in cancer |
| AKT | collective term for the serine/threonine kinases AKT1, AKT2, and AKT3 |
| ATCC | American Tissue Culture Collection |
| DSMZ | Deutsche Sammlung von Mikroorganismen und Zellkulturen |
| CRISPR | clustered regularly interspaced short palindromic repeats |
| CDX | cell line-derived xenograft |
| DMSO | dimethyl sulfoxide |
| HPβCD | 2 Hydroxypropyl Beta Cyclodextrin |
| MC | methylcellulose |
| cps | centipoise (a measure of viscosity) |
| RIKEN | Rikagaku Kenkyūjo (Institute of Physical and Chemical Research) |
| JCRB | Japanese Collection of Research Bioresources |
| CTG | CellTiter-Glo ® Luminescent Cell Viability Assay |

### Example 1: In Vitro Study of Compound 1-1 Combined with PI3K/Akt/mTOR Inhibitors in Breast Cancer Cell Lines

Five cell lines were used in this example, including CAL51, MCF7, MDA-MB-157, MDA-MB-231, and CAL51 mutant (see Table 4 for description). Compound 1-1 (Table 1) had varying concentrations from 4.1 nM to 1 uM. Compounds Alpelisib, GNE-477, HS-173, Serabelisib Everolimus, and KU-0063794 (Table 2) had varying concentrations from 41 nM to 10 uM.

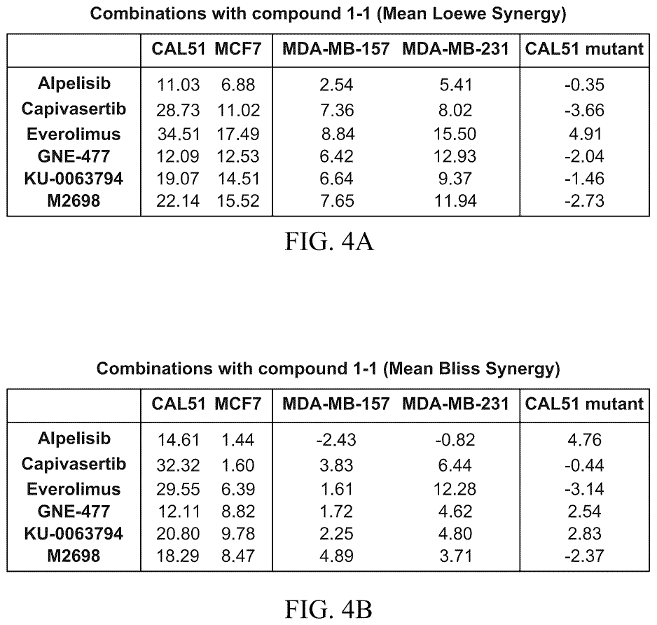

FIG. 4A shows mean Loewe synergy scores for combinations; as described above, the mean synergy score summarizes synergy scores across the entire dose response matrix. No synergistic interaction was been observed in CAL51 mutant cell line. In CAL51, MCF7, MDA-MB-157, and MDA-MB-231 cell lines, synergistic effects have been observed, e.g., compound 1-1 combined with Alpelisib (PI3K pathway inhibitors) in CAL51 cell line had the mean Loewe synergy score of 11.03 (p-value=6.39e-7). As a second example, compound 1-1 combined with Everolimus (mTOR pathway inhibitor) in CAL51 cell line had the mean Loewe synergy score of 34.51 (p-value=1.24e-12). Even after adjusting the multiple hypothesis testing, many significantly synergistic combinations were identified.

FIG. 4B shows mean Bliss synergy scores for combinations. No synergistic interaction was been observed in CAL51 mutant cell line. In CAL51, MCF7, MDA-MB-157, and MDA-MB-231 cell lines, synergistic effects were observed, e.g., compound 1-1 combined with M2698 (AKT inhibitor) in MDA-MB-231 cell line had the mean Bliss synergy score of 4.89 (p-value=1.58e-15). Many significantly synergistic combinations were identified.

### Example 2: In Vitro Study of Compounds 1-2, 1-3, 1-4, 1-5, and 1-6 Combined with PI3K/Akt/mTOR Inhibitors in Breast Cancer Cell Lines

Four cell lines were used in this example, including CAL51, MCF7, MDA-MB-157, and MDA-MB-231 (see Table 4 for description). Compounds 1-2, 1-3, 1-4, 1-5, and 1-6 (Table 1) had varying concentrations as shown in Table 5. Compounds Alpelisib, GNE-477, HS-173, Serabelisib, Capivasertib, and M2698 (Table 2) had varying concentrations from 41 nM to 10 uM.

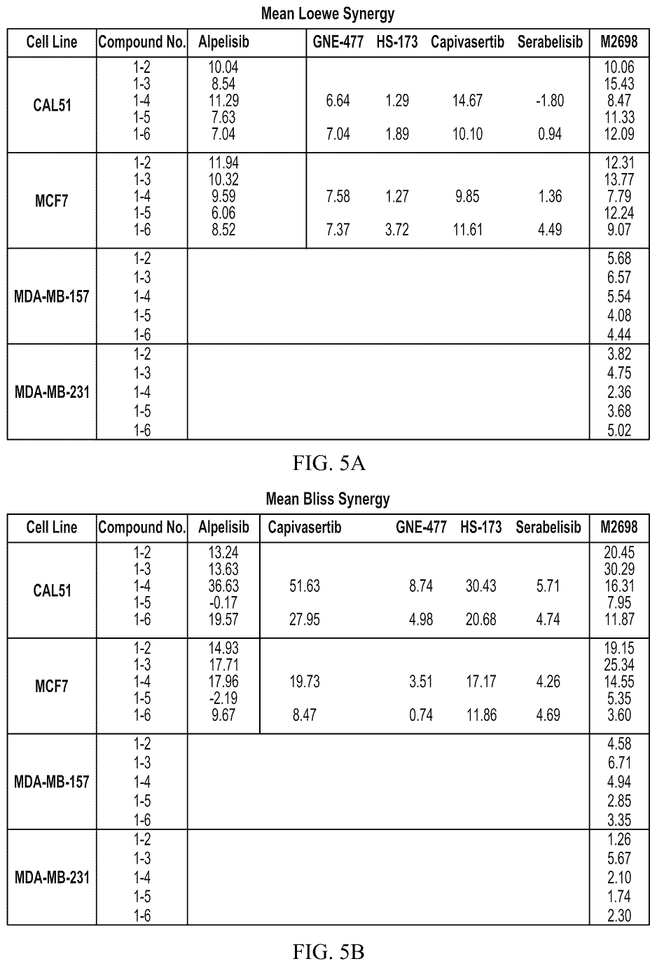

FIG. 5A shows mean Loewe synergy scores for combinations. Notable examples include the following. As an example, compound 1-4 combined with GNE-477 (PI3K inhibitors) in CAL51 cell line had the mean Loewe synergy score of 6.64 (p-value=1.19e-10). As a third example, compound 1-5 combined with M2698 (AKT inhibitor) in CAL51 cell line had the mean Loewe synergy score of 11.33 (p-value=2.59e-93)

FIG. 5B shows mean Bliss synergy scores for combinations. Notable examples include the following. Compound 1-4 combined with Alpelisib (PI3K inhibitor) in CAL51 cell line had the mean Bliss synergy score of 36.63 (p-value=1.37e-18). As a second example, compound 1-2 combined with M2698 (PI3K inhibitor) in CAL51 cell line had the mean Bliss synergy score of 20.45 (p-value=2.53e-70). As a third example, compound 1-3 combined with Alpelisib (PI3K inhibitor) in MCF7 cell line had the mean Bliss synergy score of 17.71 (p-value=6.95e-6).

### Example 3: In Vitro Study of Compound 1-1 Combined with PI3K/Akt/mTOR Inhibitors in Lung Cancer Cell Lines

Four cell lines were used in this example, including ABC-1, NCI-H1155, EBC-1, and NCI-H2023 (see Table 4 for description). Compound 1-1 (Table 1) had varying concentrations from 1.05 nM to 500 nM. Compounds GNE-477, M2698, and KU-0063794 (Table 2) had varying concentrations from 21 nM to 10 uM.

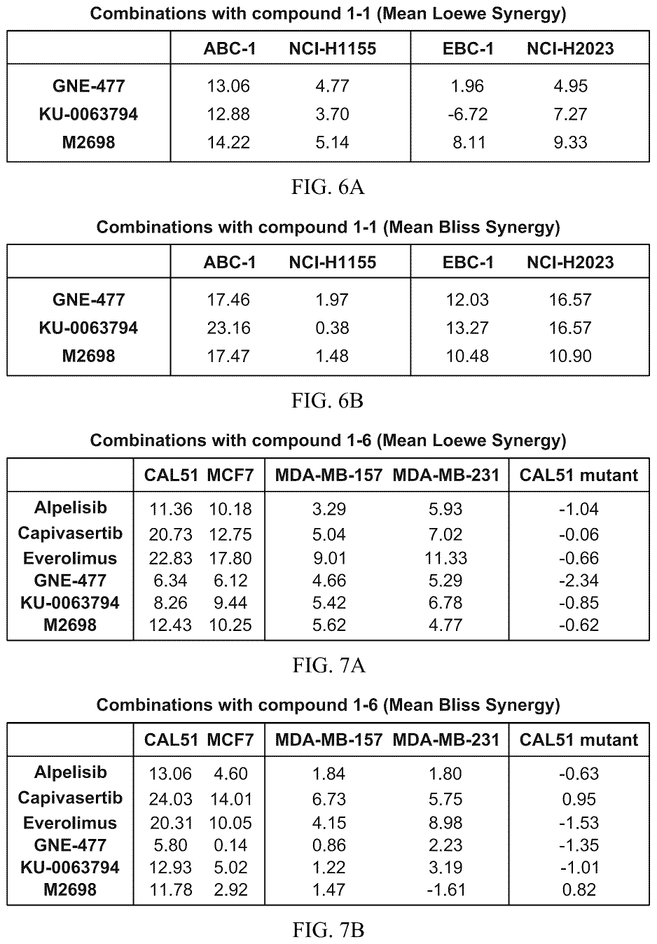

FIG. 6A shows mean Loewe synergy scores for combinations. In all four cell lines, synergistic effects have been observed.

FIG. 6B shows mean Bliss synergy scores for combinations. In all four cell lines, synergistic effects have been observed, e.g., compound 1-1 combined with GNE-477 (PI3K inhibitor) in EBC-1 cell line had the mean Bliss synergy score of 12.03 (p-value=6.15e-15). Many significantly synergistic combinations were identified.

### Example 4: In Vitro Study of Compound 1-6 Combined with PI3K/Akt/mTOR Inhibitors in Breast Cancer Cell Lines

Five cell lines were used in this example, including CAL51, MCF7, MDA-MB-157, MDA-MB-231, and CAL51 mutant (see Table 4 for description). Compound 1-6 (Table 1) had varying concentrations from 0.41 nM to 100 nM. Compounds Alpelisib, GNE-477, Capivasertib, M2698, Everolimus, and KU-0063794 (Table 2) had varying concentrations from 41 nM to 10 uM, with the exception that Everolimus in CAL51 mutant cell line had varying concentration from 4.1 nM to 1 uM.

FIG. 7A shows mean Loewe synergy scores for combinations. No synergistic interaction has been observed in CAL51 mutant cell line. In CAL51, MCF7, MDA-MB-157, and MDA-MB-231 cell lines, synergistic effects have been observed, e.g., compound 1-6 combined with M2698 (Akt inhibitor) in CAL51 cell line had the mean Loewe synergy score of 12.43 (p-value=1.13e-101). As a second example, compound 1-6 combined with compound KU-0063794 (mTOR inhibitor) in MCF7 cell line had the mean Loewe synergy score of 9.44 (p-value=3.76e-24).). As a third example, the composition 1-6 combined with compound Alpelisib (PI3K inhibitor) in CAL51 cell line had the mean Loewe synergy score of 11.36 (p-value=4.74e-21). Even after adjusting the multiple hypothesis testing, many significantly synergistic combinations were identified.

FIG. 7B shows mean Bliss synergy scores for combinations. No synergistic interaction has been observed in CAL51 mutant cell line. In CAL51, MCF7, MDA-MB-157, and MDA-MB-231 cell lines, synergistic effects have been observed, e.g., compound 1-6 combined with Everolimus (mTOR inhibitor) in MDA-MB-231 cell line had the mean Bliss synergy score of 8.98 (p-value=4.39e-14). Even after adjusting the multiple hypothesis testing, many significantly synergistic combinations were identified.

### Example 5: In Vitro Study of Compound 1-6 Combined with PI3K/Akt/mTOR Inhibitors in Lung Cancer Cell Lines

Four cell lines were used in this example, including ABC-1, NCI-H1155, EBC-1, and NCI-H2023 (see Table 4 for description). Compound 1-6 (Table 1) had varying concentrations from 0.21 nM to 100 nM. Compounds Alpelisib, GNE-477, Capivasertib, M2698, and KU-0063794 (Table 2) had varying concentrations from 21 nM to 10 uM. Everolimus (Table 2) had varying concentration from 2.1 nM to 1 uM.

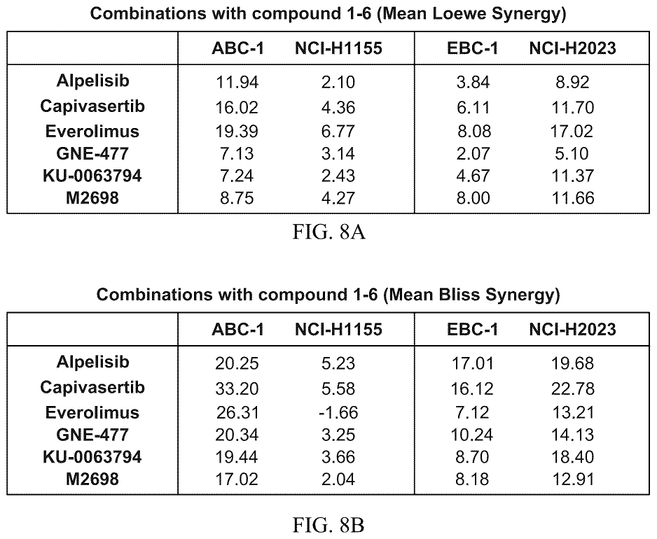

FIG. 8A shows mean Loewe synergy scores for combinations. In all four cell lines, synergistic effects have been observed, e.g., compound 1-6 combined with M2698 (AKT inhibitor) in NCI-H2023 cell line had the mean Loewe synergy score of 11.66 (p-value=3.03e-14). As a second example, compound 1-6 combined with Capivasertib (Akt inhibitor) in MCF7 cell line had the mean Loewe synergy score of 16.02 (p-value=4.89e-12). As a third example, compound 1-6 combined with Everolimus (mTOR inhibitor) in EBC-1 cell line had the mean Loewe synergy score of 8.08 (p-value=5.66e-12). Even after adjusting the multiple hypothesis testing, many significantly synergistic combinations were identified.

FIG. 8B shows mean Bliss synergy scores for combinations. In all four cell lines, synergistic effects have been observed, e.g., compound 1-6 combined with Everolimus (mTOR inhibitor) in ABC-1 cell line had the mean Bliss synergy score of 26.31 (p-value=1.60e-27). As a second example, the composition 1-6 combined with KU-0063794 (mTOR inhibitor) in NCI-H2023 cell line had the mean Bliss synergy score of 18.40 (p-value=2.52e-23). Even after adjusting the multiple hypothesis testing, many significantly synergistic combinations were identified.

TABLE 5
Numeric results

| | | | First | | Second | | | | | |
|---|---|---|---|---|---|---|---|---|---|---|
| | | | compound | | compound | | Mean Loewe | | Mean Bliss | |
| | First | Second | concentration | | concentration | | synergy score | | synergy score | |
| Cell Line | compound | compound | range (nM) | | range (nM) | | and p-value | | and p-value | |
| ABC-1 | 1-1 | GNE-477 | 1.05 | 500 | 21 | 10000 | 13.06 | 2.23e−06 | 17.46 | 4.34E−04 |
| ABC-1 | 1-1 | KU-0063794 | 1.05 | 500 | 21 | 10000 | 12.88 | 2.83e−03 | 23.16 | 1.72E−06 |
| ABC-1 | 1-1 | M2698 | 1.05 | 500 | 21 | 10000 | 14.22 | 2.07e−10 | 17.47 | 1.82E−09 |
| CAL51 | 1-1 | Alpelisib | 4.1 | 1000 | 41 | 10000 | 11.03 | 6.39e−07 | 14.61 | 1.40E−05 |
| CAL51 | 1-1 | Capivasertib | 4.1 | 1000 | 41 | 10000 | 28.73 | 4.88e−26 | 32.32 | 1.53E−21 |
| CAL51 | 1-1 | Everolimus | 4.1 | 1000 | 41 | 10000 | 34.51 | 1.24e−12 | 29.55 | 5.98E−17 |
| CAL51 | 1-1 | GNE-477 | 4.1 | 1000 | 41 | 10000 | 12.09 | 6.80e−12 | 12.11 | 3.08E−08 |
| CAL51 | 1-1 | KU-0063794 | 4.1 | 1000 | 41 | 10000 | 19.07 | 1.81e−41 | 20.80 | 3.87E−42 |
| CAL51 | 1-1 | M2698 | 4.1 | 1000 | 41 | 10000 | 22.14 | 1.73e−56 | 18.29 | 3.17E−24 |
| CAL51 mutant | 1-1 | Alpelisib | 4.1 | 1000 | 41 | 10000 | 0.35 | 8.01e−01 | 4.76 | 6.71E−04 |
| CAL51 mutant | 1-1 | Capivasertib | 4.1 | 1000 | 41 | 10000 | −3.66 | 2.48e−02 | −0.44 | 7.46E−01 |
| CAL51 mutant | 1-1 | Everolimus | 4.1 | 1000 | 4.1 | 1000 | 4.91 | 7.01e−01 | −3.14 | 2.28E−02 |
| CAL51 mutant | 1-1 | GNE-477 | 4.1 | 1000 | 41 | 10000 | −2.04 | 1.88e−01 | 2.54 | 3.36E−01 |
| CAL51 mutant | 1-1 | KU-0063794 | 4.1 | 1000 | 41 | 10000 | −1.46 | 2.63e−01 | 2.83 | 6.87E−02 |
| CAL51 mutant | 1-1 | M2698 | 4.1 | 1000 | 41 | 10000 | −2.73 | 2.57e−02 | −2.37 | 1.48E−01 |
| EBC-1 | 1-1 | GNE-477 | 1.05 | 500 | 21 | 10000 | 1.96 | 1.53e−01 | 12.03 | 6.15E−15 |
| EBC-1 | 1-1 | KU-0063794 | 1.05 | 500 | 21 | 10000 | −6.72 | 7.92e−01 | 13.27 | 3.03E−05 |
| EBC-1 | 1-1 | M2698 | 1.05 | 500 | 21 | 10000 | 8.11 | 5.59e−05 | 10.48 | 5.59E−04 |
| MCF7 | 1-1 | Alpelisib | 4.1 | 1000 | 41 | 10000 | 6.88 | 5.71e−01 | 1.44 | 8.82E−01 |
| MCF7 | 1-1 | Capivasertib | 4.1 | 1000 | 41 | 10000 | 11.02 | 4.44e−01 | 1.60 | 8.80E−01 |
| MCF7 | 1-1 | Everolimus | 4.1 | 1000 | 41 | 10000 | 17.49 | 2.17e−01 | 6.39 | 4.13E−01 |
| MCF7 | 1-1 | GNE-477 | 4.1 | 1000 | 41 | 10000 | 12.53 | 2.03e−01 | 8.82 | 2.68E−01 |
| MCF7 | 1-1 | KU-0063794 | 4.1 | 1000 | 41 | 10000 | 14.51 | 2.50e−01 | 9.78 | 3.41E−01 |
| MCF7 | 1-1 | M2698 | 4.1 | 1000 | 41 | 10000 | 15.52 | 3.23e−01 | 8.47 | 4.59E−01 |
| MDA-MB-157 | 1-1 | Alpelisib | 4.1 | 1000 | 41 | 10000 | 2.54 | 2.70e−02 | −2.43 | 2.24E−02 |
| MDA-MB-157 | 1-1 | Capivasertib | 4.1 | 1000 | 41 | 10000 | 7.36 | 9.79e−17 | 3.83 | 8.75E−02 |
| MDA-MB-157 | 1-1 | Everolimus | 4.1 | 1000 | 41 | 10000 | 8.84 | 2.45e−44 | 1.61 | 1.55E−02 |
| MDA-MB-157 | 1-1 | GNE-477 | 4.1 | 1000 | 41 | 10000 | 6.42 | 1.83e−22 | 1.72 | 2.23E−03 |
| MDA-MB-157 | 1-1 | KU-0063794 | 4.1 | 1000 | 41 | 10000 | 6.64 | 1.22e−46 | 2.25 | 3.77E−03 |
| MDA-MB-157 | 1-1 | M2698 | 4.1 | 1000 | 41 | 10000 | 7.65 | 4.96e−21 | 4.89 | 1.58E−15 |
| MDA-MB-231 | 1-1 | Alpelisib | 4.1 | 1000 | 41 | 10000 | 5.41 | 5.66e−03 | −0.82 | 7.28E−01 |
| MDA-MB-231 | 1-1 | Capivasertib | 4.1 | 1000 | 41 | 10000 | 8.02 | 2.58e−02 | 6.44 | 1.08E−01 |
| MDA-MB-231 | 1-1 | Everolimus | 4.1 | 1000 | 41 | 10000 | 15.50 | 9.01e−29 | 12.28 | 2.53E−15 |
| MDA-MB-231 | 1-1 | GNE-477 | 4.1 | 1000 | 41 | 10000 | 12.93 | 1.16e−20 | 4.62 | 1.44E−02 |
| MDA-MB-231 | 1-1 | KU-0063794 | 4.1 | 1000 | 41 | 10000 | 9.37 | 1.52e−11 | 4.80 | 1.49E−03 |
| MDA-MB-231 | 1-1 | M2698 | 4.1 | 1000 | 41 | 10000 | 11.94 | 1.40e−17 | 3.71 | 8.10E−03 |
| NCI-H1155 | 1-1 | GNE-477 | 1.05 | 500 | 21 | 10000 | 4.77 | 6.02e−10 | 1.97 | 1.00E−05 |
| NCI-H1155 | 1-1 | KU-0063794 | 1.05 | 500 | 21 | 10000 | 3.70 | 3.26e−32 | 0.38 | 7.40E−01 |
| NCI-H1155 | 1-1 | M2698 | 1.05 | 500 | 21 | 10000 | 5.14 | 3.99e−44 | 1.48 | 7.17E−03 |
| NCI-H2023 | 1-1 | GNE-477 | 1.05 | 500 | 21 | 10000 | 4.95 | 1.80e−03 | 16.57 | 1.20E−05 |
| NCI-H2023 | 1-1 | KU-0063794 | 1.05 | 500 | 21 | 10000 | 7.27 | 9.95e−10 | 16.57 | 3.41E−05 |
| NCI-H2023 | 1-1 | M2698 | 1.05 | 500 | 21 | 10000 | 9.33 | 1.09e−09 | 10.90 | 2.92E−11 |
| CAL51 | 1-2 | Alpelisib | 0.41 | 33.33 | 41 | 10000 | 10.04 | 2.45e−01 | 13.24 | 3.08E−01 |
| CAL51 | 1-2 | M2698 | 0.41 | 100 | 41 | 10000 | 10.06 | 4.05e−75 | 20.45 | 2.53E−70 |
| MCF7 | 1-2 | Alpelisib | 0.41 | 33.33 | 41 | 10000 | 11.94 | 7.61e−07 | 14.93 | 4.09E−06 |
| MCF7 | 1-2 | M2698 | 0.41 | 100 | 41 | 10000 | 12.31 | 1.23e−29 | 19.15 | 4.82E−25 |
| MDA-MB-157 | 1-2 | M2698 | 0.41 | 100 | 41 | 10000 | 5.68 | 2.19e−11 | 4.58 | 2.61E−05 |
| MDA-MB-231 | 1-2 | M2698 | 0.41 | 100 | 41 | 10000 | 3.82 | 2.70e−02 | 1.26 | 6.22E−01 |
| CAL51 | 1-3 | Alpelisib | 0.41 | 33.33 | 41 | 10000 | 8.54 | 4.57e−01 | 13.63 | 4.57E−01 |
| CAL51 | 1-3 | M2698 | 0.41 | 100 | 41 | 10000 | 15.43 | 5.14e−136 | 30.29 | 1.78E−81 |
| MCF7 | 1-3 | Alpelisib | 0.41 | 33.33 | 41 | 10000 | 10.32 | 2.96e−06 | 17.71 | 6.95E−06 |
| MCF7 | 1-3 | M2698 | 0.41 | 100 | 41 | 10000 | 13.77 | 1.13e−13 | 25.34 | 5.66E−25 |
| MDA-MB-157 | 1-3 | M2698 | 0.41 | 100 | 41 | 10000 | 6.57 | 1.41e−11 | 6.71 | 1.11E−06 |
| MDA-MB-231 | 1-3 | M2698 | 0.41 | 100 | 41 | 10000 | 4.75 | 6.27e−05 | 5.67 | 6.65E−05 |
| CAL51 | 1-4 | Alpelisib | 0.41 | 100 | 41 | 10000 | 11.29 | 1.24e−07 | 36.63 | 1.37E−18 |
| CAL51 | 1-4 | Alpelisib | 0.41 | 33.33 | 41 | 10000 | 7.63 | 4.75e−01 | 10.76 | 2.24E−01 |
| CAL51 | 1-4 | Capivasertib | 0.41 | 100 | 41 | 10000 | 14.67 | 7.98e−05 | 30.43 | 3.44E−23 |
| CAL51 | 1-4 | GNE-477 | 0.41 | 100 | 41 | 10000 | 6.64 | 1.19e−10 | 15.82 | 3.27E−16 |
| CAL51 | 1-4 | HS173 | 0.41 | 100 | 41 | 10000 | 1.29 | 1.44e−01 | 8.74 | 1.46E−31 |
| CAL51 | 1-4 | M2698 | 0.41 | 100 | 41 | 10000 | 8.47 | 1.38e−18 | 16.31 | 1.18E−22 |
| CAL51 | 1-4 | Serabelisib | 0.41 | 100 | 41 | 10000 | −1.80 | 2.97e−01 | 5.71 | 3.67E−03 |
| MCF7 | 1-4 | Alpelisib | 0.41 | 33.33 | 41 | 10000 | 7.90 | 1.27e−04 | 9.41 | 4.28E−04 |
| MCF7 | 1-4 | Alpelisib | 0.41 | 100 | 41 | 10000 | 9.59 | 6.97e−09 | 17.96 | 1.01E−08 |
| MCF7 | 1-4 | Capivasertib | 0.41 | 100 | 41 | 10000 | 9.85 | 7.44e−06 | 17.17 | 1.71E−08 |
| MCF7 | 1-4 | GNE-477 | 0.41 | 100 | 41 | 10000 | 7.58 | 7.55e−07 | 9.39 | 3.55E−14 |
| MCF7 | 1-4 | HS173 | 0.41 | 100 | 41 | 10000 | 1.27 | 2.83e−01 | 3.51 | 9.07E−03 |
| MCF7 | 1-4 | M2698 | 0.41 | 100 | 41 | 10000 | 7.79 | 2.38e−07 | 14.55 | 9.26E−13 |
| MCF7 | 1-4 | Serabelisib | 0.41 | 100 | 41 | 10000 | 1.36 | 1.88e−01 | 4.26 | 2.33E−05 |
| MDA-MB-157 | 1-4 | M2698 | 0.41 | 100 | 41 | 10000 | 5.54 | 5.32e−06 | 4.94 | 1.09E−03 |
| MDA-MB-231 | 1-4 | M2698 | 0.41 | 100 | 41 | 10000 | 2.36 | 1.64e−01 | 2.10 | 4.77E−01 |
| CAL51 | 1-5 | Alpelisib | 0.41 | 33.33 | 41 | 10000 | 7.63 | 5.10e−01 | −0.17 | 9.88E−01 |
| CAL51 | 1-5 | M2698 | 0.41 | 100 | 41 | 10000 | 11.33 | 2.59e−93 | 7.95 | 2.19E−05 |
| MCF7 | 1-5 | Alpelisib | 0.41 | 33.33 | 41 | 10000 | 6.06 | 2.83e−02 | −2.19 | 5.04E−01 |
| MCF7 | 1-5 | M2698 | 0.41 | 100 | 41 | 10000 | 12.24 | 1.63e−11 | 5.35 | 4.90E−03 |
| MDA-MB-157 | 1-5 | M2698 | 0.41 | 100 | 41 | 10000 | 4.08 | 4.85e−04 | 2.85 | 5.85E−02 |
| MDA-MB-231 | 1-5 | M2698 | 0.41 | 100 | 41 | 10000 | 3.68 | 9.90e−03 | 1.74 | 4.86E−01 |
| ABC-1 | 1-6 | Alpelisib | 0.21 | 100 | 21 | 10000 | 11.94 | 2.51e−09 | 20.25 | 2.91E−11 |
| ABC-1 | 1-6 | Capivasertib | 0.21 | 100 | 21 | 10000 | 16.0 | 4.89e−12 | 33.20 | 1.91E−28 |
| ABC-1 | 1-6 | Everolimus | 0.21 | 100 | 2.1 | 1000 | 19.39 | 6.18e−26 | 26.31 | 1.60E−27 |
| ABC-1 | 1-6 | GNE-477 | 0.21 | 100 | 21 | 10000 | 7.13 | 1.40e−09 | 20.34 | 4.42E−14 |
| ABC-1 | 1-6 | KU-0063794 | 0.21 | 100 | 21 | 10000 | 7.24 | 8.27e−04 | 19.44 | 2.39E−06 |
| ABC-1 | 1-6 | M2698 | 0.21 | 100 | 21 | 10000 | 8.75 | 2.05e−03 | 17.02 | 9.35E−04 |
| CAL51 | 1-6 | Alpelisib | 0.41 | 100 | 41 | 10000 | 11.36 | 4.74e−21 | 13.06 | 4.89E−21 |
| CAL51 | 1-6 | Alpelisib | 0.41 | 100 | 41 | 10000 | 7.04 | 1.01e−07 | 19.57 | 4.29E−16 |
| CAL51 | 1-6 | Alpelisib | 0.41 | 33.33 | 41 | 10000 | 7.89 | 3.73e−01 | 4.15 | 7.22E−01 |
| CAL51 | 1-6 | Capivasertib | 0.41 | 100 | 41 | 10000 | 20.73 | 2.92e−10 | 24.03 | 6.66E−61 |
| CAL51 | 1-6 | Capivasertib | 0.41 | 100 | 41 | 10000 | 10.10 | 7.46e−19 | 20.68 | 7.22E−63 |
| CAL51 | 1-6 | Everolimus | 0.41 | 100 | 41 | 10000 | 22.83 | 7.97e−08 | 20.31 | 7.19E−71 |
| CAL51 | 1-6 | GNE-477 | 0.41 | 100 | 41 | 10000 | 6.34 | 9.86e−05 | 5.80 | 5.13E−03 |
| CAL51 | 1-6 | HS173 | 0.41 | 100 | 41 | 10000 | 1.89 | 1.22e−05 | 4.98 | 1.31E−21 |
| CAL51 | 1-6 | KU-0063794 | 0.41 | 100 | 41 | 10000 | 8.26 | 3.16e−06 | 12.93 | 1.17E−14 |
| CAL51 | 1-6 | M2698 | 0.41 | 100 | 41 | 10000 | 12.43 | 1.13e−101 | 11.78 | 2.97E−41 |
| CAL51 | 1-6 | M2698 | 0.41 | 100 | 41 | 10000 | 12.09 | 6.95e−62 | 11.87 | 2.39E−34 |
| CAL51 | 1-6 | Serabelisib | 0.41 | 100 | 41 | 10000 | 0.94 | 5.48e−01 | 4.74 | 1.40E−03 |
| CAL51 mutant | 1-6 | Alpelisib | 0.41 | 100 | 41 | 10000 | −1.04 | 6.46e−01 | −0.63 | 8.24E−01 |
| CAL51 mutant | 1-6 | Capivasertib | 0.41 | 100 | 41 | 10000 | −0.06 | 9.73e−01 | 0.95 | 4.86E−01 |
| CAL51 mutant | 1-6 | Everolimus | 0.41 | 100 | 4.1 | 1000 | −0.66 | 6.06e−01 | −1.53 | 3.62E−01 |
| CAL51 mutant | 1-6 | GNE-477 | 0.41 | 100 | 41 | 10000 | −2.34 | 1.55e−02 | −1.35 | 1.26E−01 |
| CAL51 mutant | 1-6 | KU-0063794 | 0.41 | 100 | 41 | 10000 | −0.85 | 5.92e−01 | −1.01 | 5.90E−01 |
| CAL51 mutant | 1-6 | M2698 | 0.41 | 100 | 41 | 10000 | −0.62 | 6.36e−01 | 0.82 | 3.82E−01 |
| EBC-1 | 1-6 | Alpelisib | 0.21 | 100 | 21 | 10000 | 3.84 | 4.10e−04 | 17.01 | 2.81E−12 |
| EBC-1 | 1-6 | Capivasertib | 0.21 | 100 | 21 | 10000 | 6.11 | 3.50e−03 | 16.12 | 7.94E−14 |
| EBC-1 | 1-6 | Everolimus | 0.21 | 100 | 2.1 | 1000 | 8.08 | 5.66e−12 | 7.12 | 3.45E−05 |
| EBC-1 | 1-6 | GNE-477 | 0.21 | 100 | 21 | 10000 | 2.07 | 2.04e−01 | 10.24 | 2.85E−06 |
| EBC-1 | 1-6 | KU-0063794 | 0.21 | 100 | 21 | 10000 | 4.67 | 1.63e−06 | 8.70 | 1.92E−07 |
| EBC-1 | 1-6 | M2698 | 0.21 | 100 | 21 | 10000 | 8.00 | 6.90e−09 | 8.18 | 1.60E−10 |
| MCF7 | 1-6 | Alpelisib | 0.41 | 100 | 41 | 10000 | 10.18 | 3.61e−25 | 4.60 | 2.85E−04 |
| MCF7 | 1-6 | Alpelisib | 0.41 | 100 | 41 | 10000 | 8.52 | 1.02e−07 | 9.67 | 7.37E−04 |
| MCF7 | 1-6 | Alpelisib | 0.41 | 33.33 | 41 | 10000 | 9.38 | 5.40e−05 | 3.23 | 1.44E−01 |
| MCF7 | 1-6 | Capivasertib | 0.41 | 100 | 41 | 10000 | 12.75 | 1.30e−04 | 14.01 | 2.91E−07 |
| MCF7 | 1-6 | Capivasertib | 0.41 | 100 | 41 | 10000 | 11.61 | 1.37e−05 | 11.86 | 1.63E−05 |
| MCF7 | 1-6 | Everolimus | 0.41 | 100 | 41 | 10000 | 17.80 | 1.14e−20 | 10.05 | 5.84E−06 |
| MCF7 | 1-6 | GNE-477 | 0.41 | 100 | 41 | 10000 | 6.12 | 6.60e−20 | 0.14 | 8.48E−01 |
| MCF7 | 1-6 | HS173 | 0.41 | 100 | 41 | 10000 | 3.72 | 6.55e−04 | 0.74 | 5.35E−01 |
| MCF7 | 1-6 | KU-0063794 | 0.41 | 100 | 41 | 10000 | 9.44 | 3.76e−24 | 5.02 | 7.04E−11 |
| MCF7 | 1-6 | M2698 | 0.41 | 100 | 41 | 10000 | 10.25 | 2.34e−33 | 2.92 | 6.73E−03 |
| MCF7 | 1-6 | M2698 | 0.41 | 100 | 41 | 10000 | 9.07 | 3.44e−08 | 3.60 | 5.97E−02 |
| MCF7 | 1-6 | Serabelisib | 0.41 | 100 | 41 | 10000 | 4.49 | 9.17e−07 | 4.69 | 8.36E−03 |
| MDA-MB-157 | 1-6 | Alpelisib | 0.41 | 100 | 41 | 10000 | 3.29 | 2.17e−02 | 1.84 | 1.85E−01 |
| MDA-MB-157 | 1-6 | Capivasertib | 0.41 | 100 | 41 | 10000 | 5.04 | 9.39e−03 | 6.73 | 1.50E−05 |
| MDA-MB-157 | 1-6 | Everolimus | 0.41 | 100 | 41 | 10000 | 9.01 | 1.97e−08 | 4.15 | 2.37E−03 |
| MDA-MB-157 | 1-6 | GNE-477 | 0.41 | 100 | 41 | 10000 | 4.66 | 3.87e−13 | 0.86 | 2.72E−01 |
| MDA-MB-157 | 1-6 | KU-0063794 | 0.41 | 100 | 41 | 10000 | 5.42 | 6.10e−14 | 1.22 | 2.42E−02 |
| MDA-MB-157 | 1-6 | M2698 | 0.41 | 100 | 41 | 10000 | 5.62 | 1.56e−21 | 1.47 | 1.43E−01 |
| MDA-MB-157 | 1-6 | M2698 | 0.41 | 100 | 41 | 10000 | 4.44 | 2.79e−02 | 3.35 | 1.17E−01 |
| MDA-MB-231 | 1-6 | Alpelisib | 0.41 | 100 | 41 | 10000 | 5.93 | 3.31e−16 | 1.80 | 1.69E−01 |
| MDA-MB-231 | 1-6 | Capivasertib | 0.41 | 100 | 41 | 10000 | 7.02 | 1.49e−06 | 5.75 | 6.30E−03 |
| MDA-MB-231 | 1-6 | Everolimus | 0.41 | 100 | 41 | 10000 | 11.33 | 9.07e−16 | 8.98 | 4.39E−14 |
| MDA-MB-231 | 1-6 | GNE-477 | 0.41 | 100 | 41 | 10000 | 5.29 | 3.46e−04 | 2.23 | 4.73E−03 |
| MDA-MB-231 | 1-6 | KU-0063794 | 0.41 | 100 | 41 | 10000 | 6.78 | 3.67e−08 | 3.19 | 1.07E−02 |
| MDA-MB-231 | 1-6 | M2698 | 0.41 | 100 | 41 | 10000 | 4.77 | 3.22e−28 | −1.61 | 1.21E−01 |
| MDA-MB-231 | 1-6 | M2698 | 0.41 | 100 | 41 | 10000 | 5.02 | 3.75e−02 | 2.30 | 3.09E−01 |
| NCI-H1155 | 1-6 | Alpelisib | 0.21 | 100 | 21 | 10000 | 2.10 | 1.79e−01 | 5.23 | 1.01E−02 |
| NCI-H1155 | 1-6 | Capivasertib | 0.21 | 100 | 21 | 10000 | 4.36 | 1.43e−08 | 5.58 | 4.14E−11 |
| NCI-H1155 | 1-6 | Everolimus | 0.21 | 100 | 2.1 | 1000 | 6.77 | 7.84e−03 | −1.66 | 1.08E−02 |
| NCI-H1155 | 1-6 | GNE-477 | 0.21 | 100 | 21 | 10000 | 3.14 | 5.25e−04 | 3.25 | 2.68E−02 |
| NCI-H1155 | 1-6 | KU-0063794 | 0.21 | 100 | 21 | 10000 | 2.43 | 3.73e−02 | 3.66 | 4.54E−02 |
| NCI-H1155 | 1-6 | M2698 | 0.21 | 100 | 21 | 10000 | 4.27 | 1.74e−12 | 2.04 | 6.95E−03 |
| NCI-H2023 | 1-6 | Alpelisib | 0.21 | 100 | 21 | 10000 | 8.92 | 1.09e−08 | 19.68 | 1.02E−10 |
| NCI-H2023 | 1-6 | Capivasertib | 0.21 | 100 | 21 | 10000 | 11.70 | 8.71e−10 | 22.78 | 8.30E−11 |
| NCI-H2023 | 1-6 | Everolimus | 0.21 | 100 | 2.1 | 1000 | 17.02 | 4.35e−18 | 13.21 | 2.35E−11 |
| NCI-H2023 | 1-6 | GNE-477 | 0.21 | 100 | 21 | 10000 | 5.10 | 4.75e−03 | 14.13 | 3.84E−05 |
| NCI-H2023 | 1-6 | KU-0063794 | 0.21 | 100 | 21 | 10000 | 11.37 | 1.29e−01 | 18.40 | 2.52E−23 |
| NCI-H2023 | 1-6 | M2698 | 0.21 | 100 | 21 | 10000 | 11.66 | 3.03e−14 | 12.91 | 7.15E−09 |

### Example 6. In Vitro Study of Compound 1-1 in Combination with PI3K/Akt/mTOR Inhibitors in Non-Small Cell Lung Cancer (NSCLC) Cell Lines

### Methods:

The study was conducted by Shanghai ChemPartner Co., Ltd (Study Number CPB-P21-25329). Details on the 6 NSCLC cell lines that were part of the experiment are listed in Table 6A.

TABLE 6A
Information on the 6 NSCLC cell lines used in the study. Biomarker
status indicates whether cells are characterized by high N-MYC
mRNA expression and/or a neuroendocrine (NE) phenotype. High N-
MYC mRNA expression is associated with heightened sensitivity to
compound 1-1 as single agent. Seeding density indicates the number
of cells seeded per well on the day before compound addition.

| | | Catalog | | Seeding |
|---|---|---|---|---|
| Cell line | Supplier | number | Biomarker status | density |
| NCI-H2106 | ATCC | CRL-5923 | N-MYC high expression, | 6000 |
| | | | neuroendocrine | |
| ABC-1 | JCRB | JCRB0815 | N-MYC high expression | 1500 |
| NCI-H1770 | ATCC | CRL-5893 | N-MYC high expression, | 4000 |
| | | | neuroendocrine | |
| NCI-H441 | ATCC | HTB-174 | | 1800 |
| EBC-1 | RIKEN | RBRC- | | 1200 |
| | | RCB1965 | | |
| NCI-H2023 | ATCC | CRL-5912 | | 1000 |

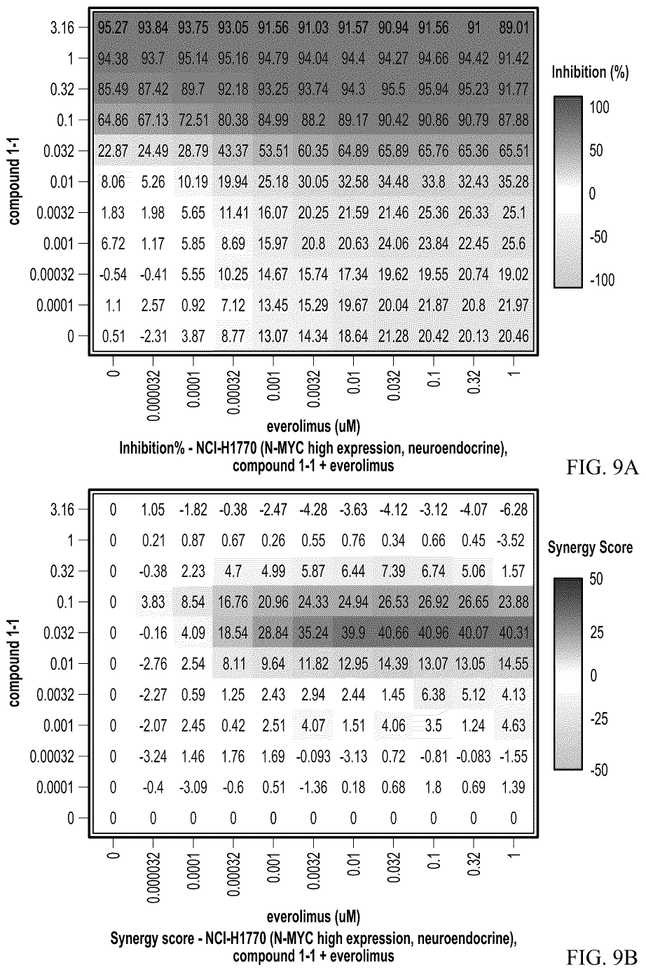

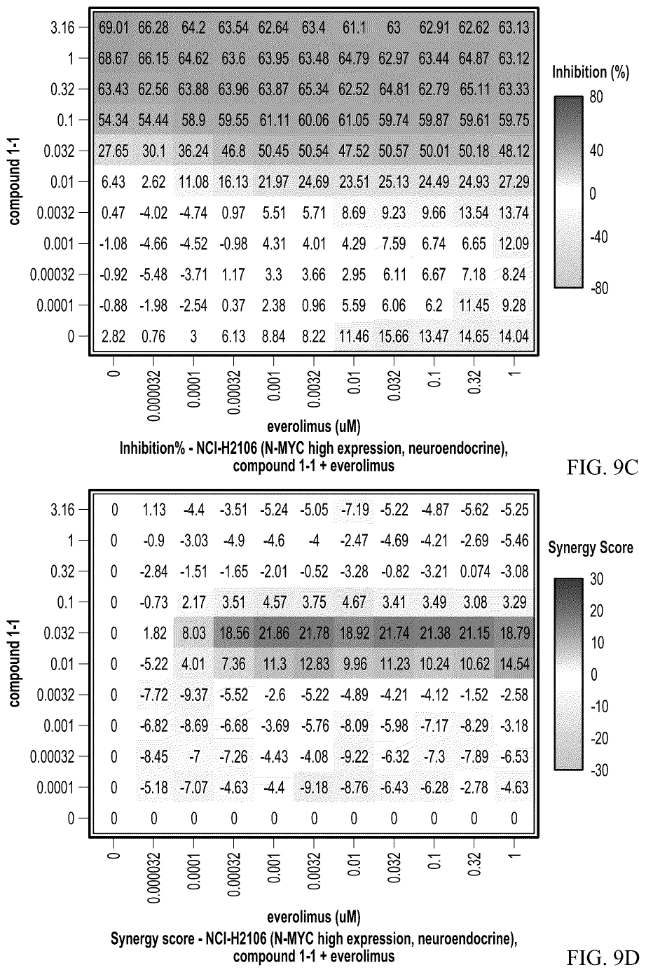

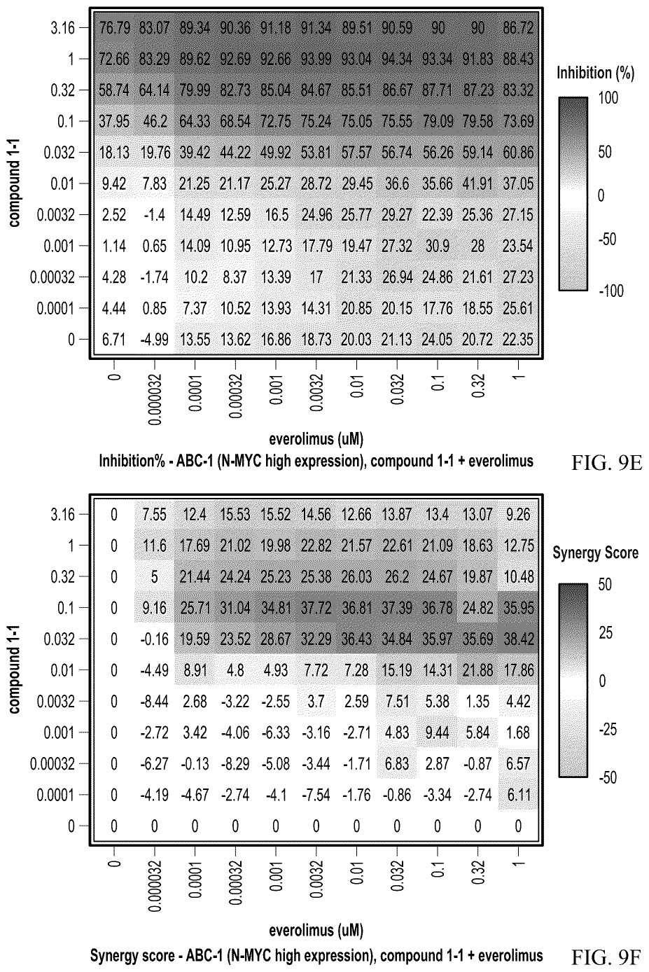

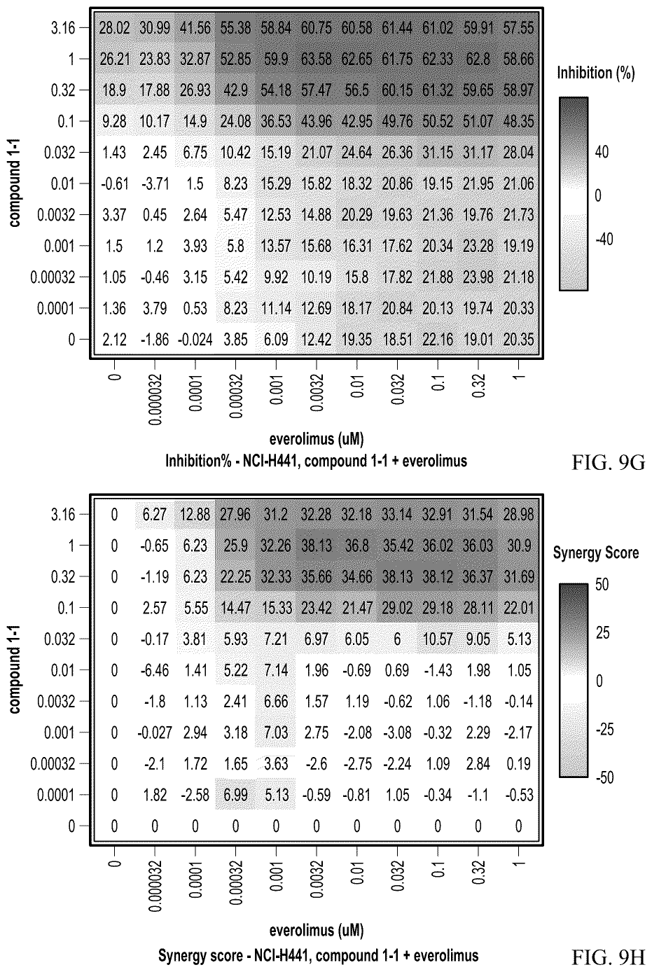

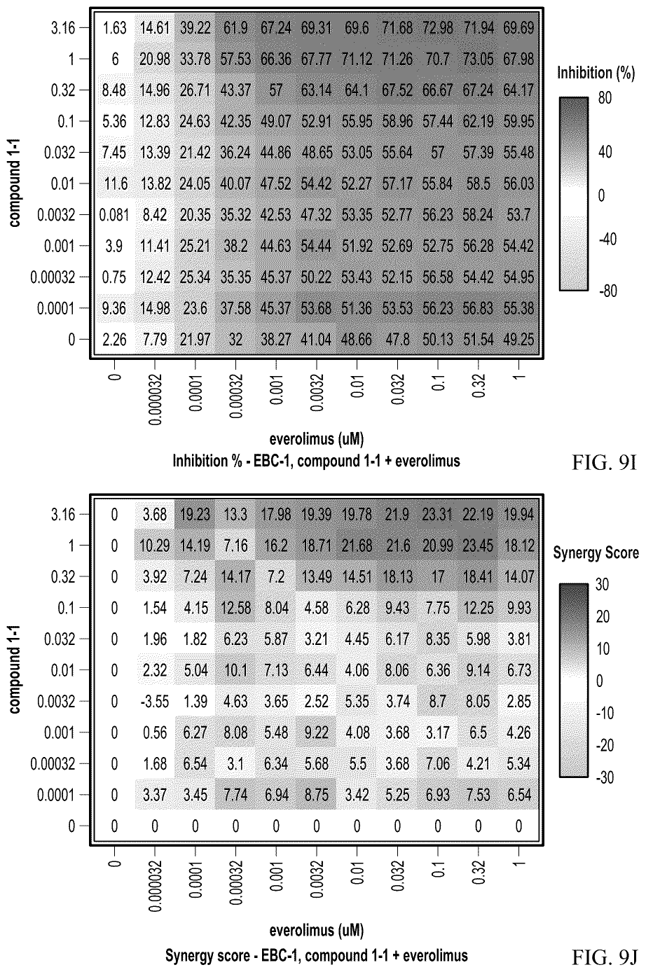

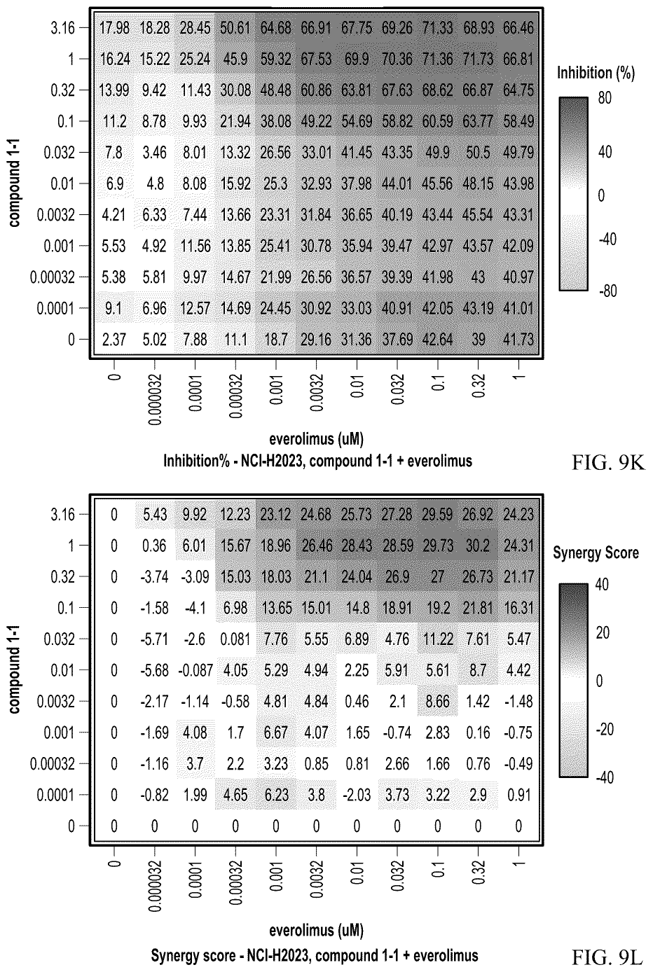

Cells were cultured in the media recommended by the supplier. Combination experiments were conducted in a similar way as described before, but with some changes in the protocol. In brief, the 6 non-small cell lung cancer cell lines were exposed to a concentration matrix of compound 1-1 and each of the following compounds in pairwise combinations: everolimus, capivasertib, buparlisib, alpelisib (see Table 6B and FIG. 9A-9L). Each concentrations matrix consisted of 11×11 points (DMSO and a 10-point half-log dilution series of compound 1-1 in one dimension and the respective combination partner in the other dimension, as exemplified in FIG. 9A-9L). Compound 1-1 concentrations ranged from 0.1 nM to 3.16 uM (micromolar). Everolimus concentrations ranged from 0.032 nM to 1 μM. Capivasertib, buparlisib and alpelisib concentrations ranged from 1 nM to 31.62 μM.

On the day before compound addition, cells were seeded in 384-well-plates (white with clear flat bottom) at the densities indicated in Table 6 in 40 μL of growth medium. The next day, compounds were dispensed from 10 mM stocks using a HP D300 dispenser according to the concentration matrices described above. For each cell line, the compound concentration matrices were dispensed twice as side-by-side duplicates on one 384-well-plate. In addition, each plate contained 14 wells with only growth medium and 44 wells with cells treated with DMSO alone to calculate the average background and maximal signals, respectively. The final DMSO concentration was normalized to 0.2% across the plates. Plates were then incubated for 72 hours. CellTiter-Glo® Luminescent Cell Viability Assay (CTG, Promega G7570) reagent was prepared according to the manufacturer's instructions. Plates were equilibrated to room temperature for approximately 30 minutes, 25 μL CTG reagent was added to each well and plates were mixed on an orbital shaker for 10 minutes to allow for cell lysis. After further incubation for 20 minutes at room temperature, the clear bottoms of the plates were pasted with white back seal and luminescence was read on an EnVision plate reader (measurement time 0.1 seconds).

Data were analyzed at Monte Rosa Therapeutics Inc using the R package “synergyfinder” as before using “Loewe additivity” as a reference model and applying the baseline correction. % inhibition was calculated as follows: (a) the average background CTG signal observed in wells with only growth medium was subtracted from the CTG signal of all other wells, (b) “vehicle” was defined as the average CTG signal across the 44 extra wells outside the matrix that contained cells only treated with DMSO, (c) for each well inside the matrix with a CTG signal X the “% inhibition” was calculated as 100*(1−X/vehicle). The “% inhibition” values displayed in FIGS. 9 to 11 are the average of 2 replicates.

### Results:

In vitro concentration matrix experiments were conducted to test whether combinations of the GSPT1 molecular glue degrader compound 1-1 and agents targeting the PI3K/AKT/mTOR pathway can synergistically reduce viability of NSCLC cells (Table 6A). The full set of data is summarized in Table 6B, where the average Loewe synergy scores are listed.

TABLE 6B
Mean Loewe synergy scores of compound 1-1 combined with PI3K/Akt/mTOR
inhibitors in treating non-small cell lung cancer (NSCLC) cell lines

| | | NSCLC cell lines | | | | | |
|---|---|---|---|---|---|---|---|
| | PI3K/Akt/ | NCI- | NCI- | | NCI- | | NCI- |
| target | mTOR inhibitor | H1770 | H2106 | ABC1 | H441 | EBC1 | H2023 |
| mTOR | everolimus | 6.49 | −0.49 | 12.04 | 11.14 | 8.69 | 8.62 |
| AKT | capivasertib | 3.31 | 3.77 | 5.4 | 4.4 | 4.03 | 3.86 |
| Pan-PI3K | buparlisib | 0.21 | −0.48 | 1.14 | 0.24 | 3.27 | 2.5 |
| PI3K alpha | alpelisib | 1.7 | −0.51 | 4.95 | 5.23 | 0.38 | 1.1 |

In most cell lines, the combination of compound 1-1 with the mTOR inhibitor everolimus resulted in the highest mean synergy scores (Table 6B). Of note, high synergy scores for this combination were sometimes only detected in a relatively narrow range of concentrations of compound 1-1 (FIG. 9A-9L), which is not always reflected in the mean synergy score across the matrix.

FIG. 9A-9F indicate that the combination of compound 1-1 and everolimus is synergistic in all three tested high N-MYC expressing cell lines within a certain range of concentrations that is centered around 0.032 to 0.1 μM of compound 1-1.

The single agent effect of compound 1-1 was smaller in cell lines without high N-MYC expression (FIG. 9G-9L). A region of synergy with everolimus was observed in these three cell lines as well but shifted towards higher compound 1-1 concentrations centering around 0.32 to 1 μM.

### Example 7. In Vitro Study of Compound 1-1 Combined with PI3K/Akt/mTOR Inhibitors in Small Cell Lung Cancer (SCLC) Cell Lines

### Methods:

The study was conducted by Shanghai ChemPartner Co., Ltd (Study Number CPB-P21-25329). Details on the 5 SCLC cell lines that were part of the experiment are listed in Table 7A.

TABLE 7A
Information on the 5 SCLC cell lines used in the study. Biomarker
status indicates whether cells are characterized by high
L-MYC or N-MYC mRNA expression and/or neuroendocrine phenotype.
High L-MYC or N-MYC mRNA expression are associated with
heightened sensitivity to compound 1-1 as single agent.
Seeding density indicates the number of cells seeded per
well on the day before compound addition.

| | | Catalog | | Seeding |
|---|---|---|---|---|
| Cell line | Supplier | number | Biomarker status | density |
| NCI-H1836 | ATCC | CRL-5898 | L-MYC high expression, | 6000 |
| | | | neuroendocrine | |
| NCI-H1876 | ATCC | CRL-5902 | L-MYC high expression, | 8000 |
| | | | neuroendocrine | |
| NCI-H209 | ATCC | HTB-172 | L-MYC high expression, | 4000 |
| | | | neuroendocrine | |
| NCI-H526 | ATCC | CRL-5811 | N-MYC high expression, | 3000 |
| | | | neuroendocrine | |
| NCI-H69 | ATCC | HTB-119 | N-MYC high expression, | 3000 |
| | | | neuroendocrine | |

Combination experiments were performed as described in Example 6.

### Results:

In vitro concentration matrix experiments were conducted to test whether combinations of the GSPT1 molecular glue degrader compound 1-1 and agents targeting the PI3K/AKT/mTOR pathway can synergistically reduce viability of SCLC cells (Table 7A). The full set of data is summarized in Table 7B, where the mean Loewe synergy scores are listed.

TABLE 7B
Mean Loewe synergy scores of compound 1-1
combined with PI3K/Akt/mTOR inhibitors in
small cell lung cancer (SCLC) cell lines

| | | SCLC cell lines | | | | |
|---|---|---|---|---|---|---|
| | PI3K/Akt/mTOR | NCI- | NCI- | NCI- | NCI- | |
| target | inhibitor | H1836 | H1876 | H209 | H526 | NCI-H69 |
| mTOR | everolimus | 1.58 | −0.29 | 6.14 | 6.49 | 9.46 |
| AKT | capivasertib | 1.9 | 0.7 | 0.85 | 4.3 | 7.38 |
| Pan- | buparlisib | 1.64 | 0.017 | 0.78 | −0.21 | 4.09 |
| PI3K | | | | | | |
| PI3K | alpelisib | 1.69 | 1.64 | 1.49 | 3.7 | 5.05 |
| alpha | | | | | | |

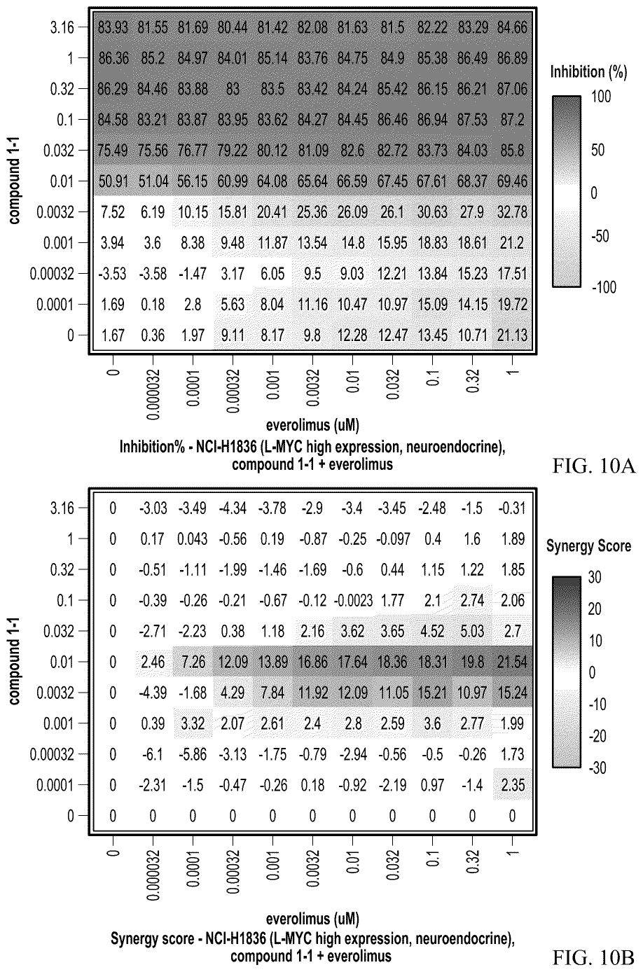

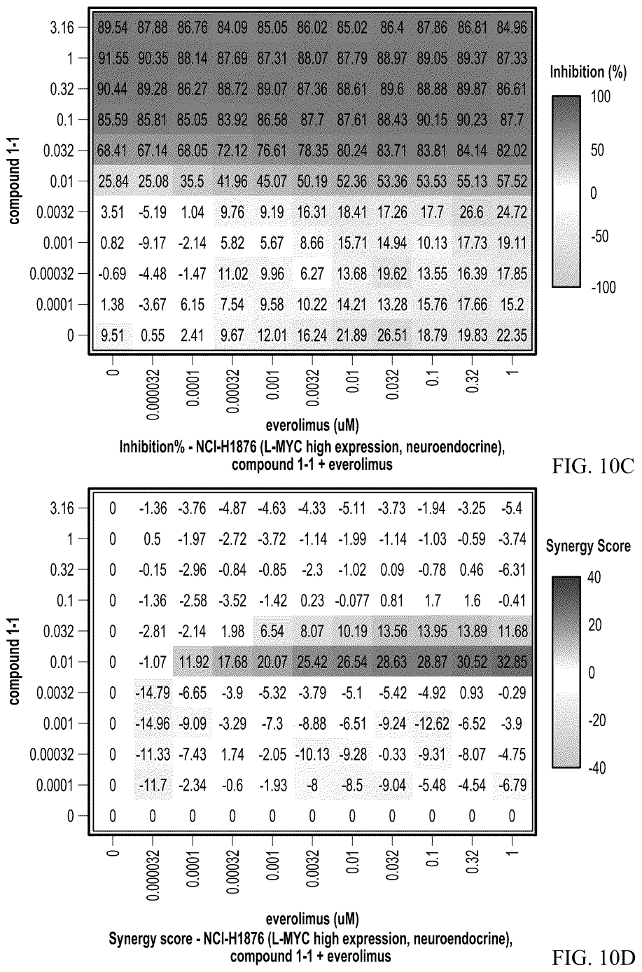

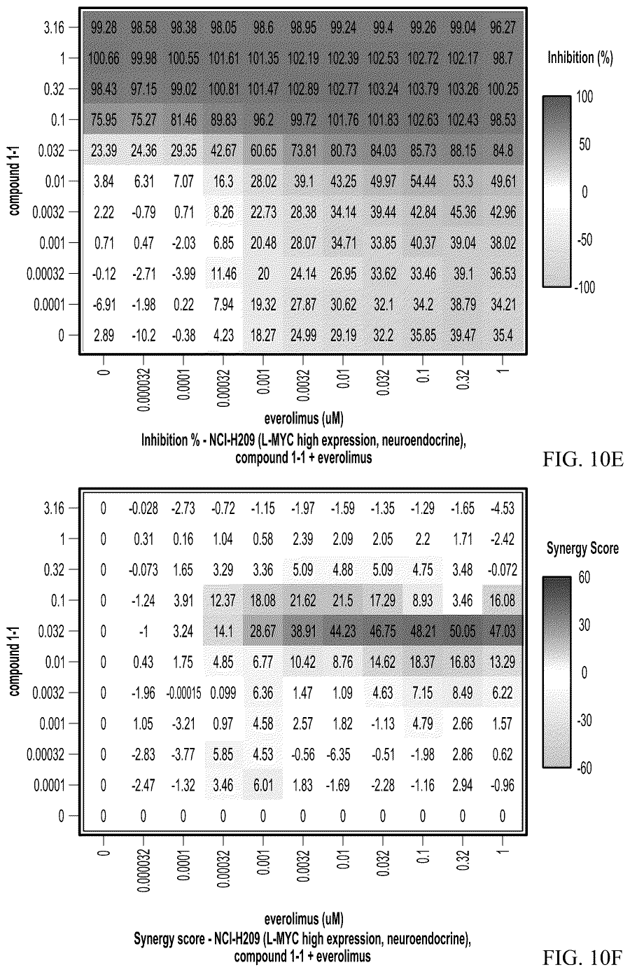

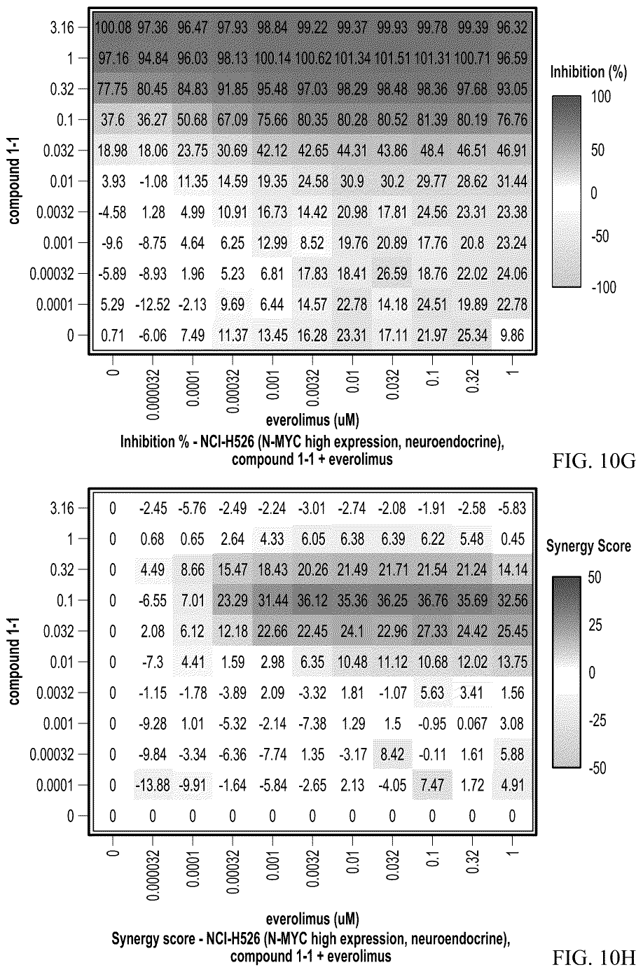

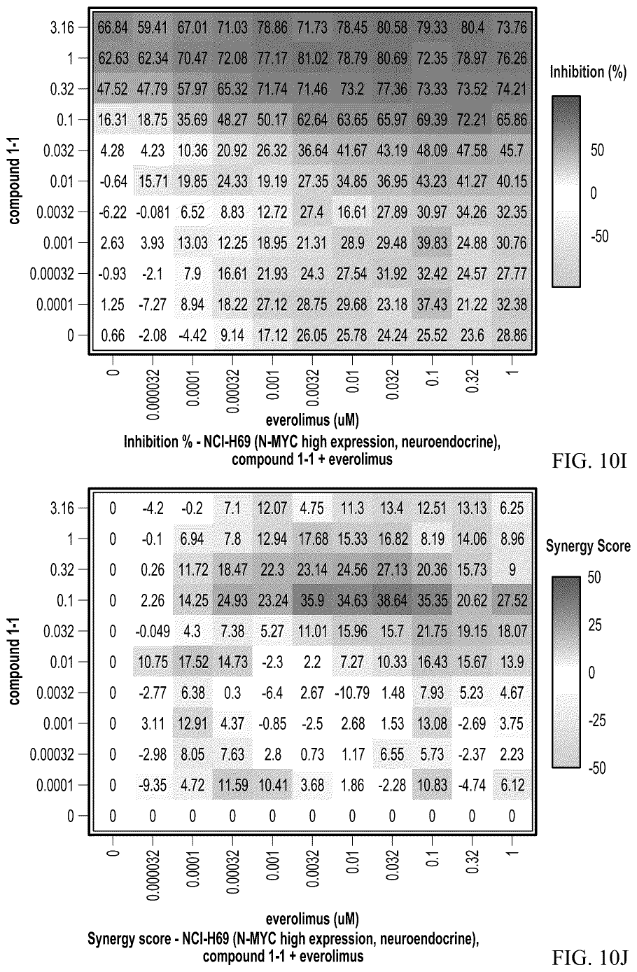

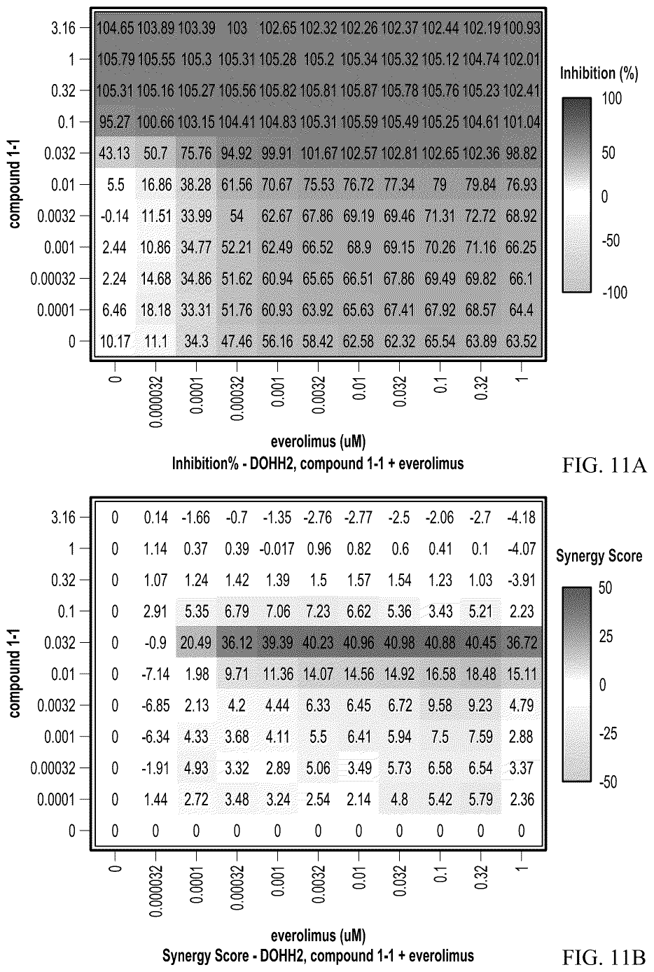

The combination of compound 1-1 with the mTOR inhibitor everolimus resulted in the highest mean synergy scores in 3 out of 5 cell lines (Table 7B), but synergy was on average less pronounced than in NSCLC (Table 6B). High synergy scores for this combination were only detected in a relatively narrow range of concentrations of compound 1-1 for the majority of tested SCLC cell lines, which was largely a consequence of the marked single agent activity of compound 1-1 (FIG. 10A-10J).

### Example 8. In Vitro Study of Compound 1-1 Combined with PI3K/Akt/mTOR Inhibitors in Lymphoma Cell Lines

### Methods:

The study was conducted by Shanghai ChemPartner Co., Ltd (Study Number CPB-P21-25329). Details on the 5 lymphoma cell lines that were part of the experiment are listed in Table 8A.

TABLE 8A
Information on the 5 lymphoma cancer cell lines used in
the study. Seeding density indicates the number of cells
seeded per well on the day before compound addition.

| Cell line | Supplier | Catalog number | Seeding density |
|---|---|---|---|
| DOHH-2 | DSMZ | ACC-47 | 4000 |
| DB | ATCC | CRL-2289 | 2000 |
| WSU-DLCL2 | DSMZ | ACC-575 | 4000 |
| RAJI | ATCC | CCL-86 | 2000 |
| SU-DHL-6 | ATCC | CRL-2959 | 4000 |

Combination experiments were performed as described in Example 6. In addition to the compounds that were part of Example 6, compound 1-1 was also combined with idelalisib (concentration range: 1 nM to 3.16 uM).

### Results:

In vitro concentration matrix experiments were conducted to test whether combinations of the GSPT1 molecular glue degrader compound 1-1 and agents targeting the PI3K/AKT/mTOR pathway can synergistically reduce viability of lymphoma cells (Table 8A). The full set of data is summarized in Table 8B, where the mean Loewe synergy scores are listed.

TABLE 8B
Mean Loewe synergy scores of compound 1-1 combined with
PI3K/Akt/mTOR inhibitors in lymphoma cell lines

| | PI3K/Akt/ | Lymphoma cell lines | | | | |
|---|---|---|---|---|---|---|
| | mTOR | | | WSU- | | |
| target | inhibitor | DOHH2 | DB | DLCL2 | RAJI | SUDHL6 |
| mTOR | everolimus | 6.48 | 5.81 | 8.29 | 14.66 | 12.05 |
| AKT | capivasertib | 2.89 | 0.29 | 0.95 | 2.97 | −1.92 |
| Pan-PI3K | buparlisib | 1.11 | 1.26 | −3.1 | 1.34 | −2.5 |
| PI3K alpha | alpelisib | 1.59 | −2.27 | −2.69 | 2.05 | −6.28 |
| PI3K delta | Idelaisib | 4.04 | −3.25 | −2.08 | 3.47 | 5.75 |

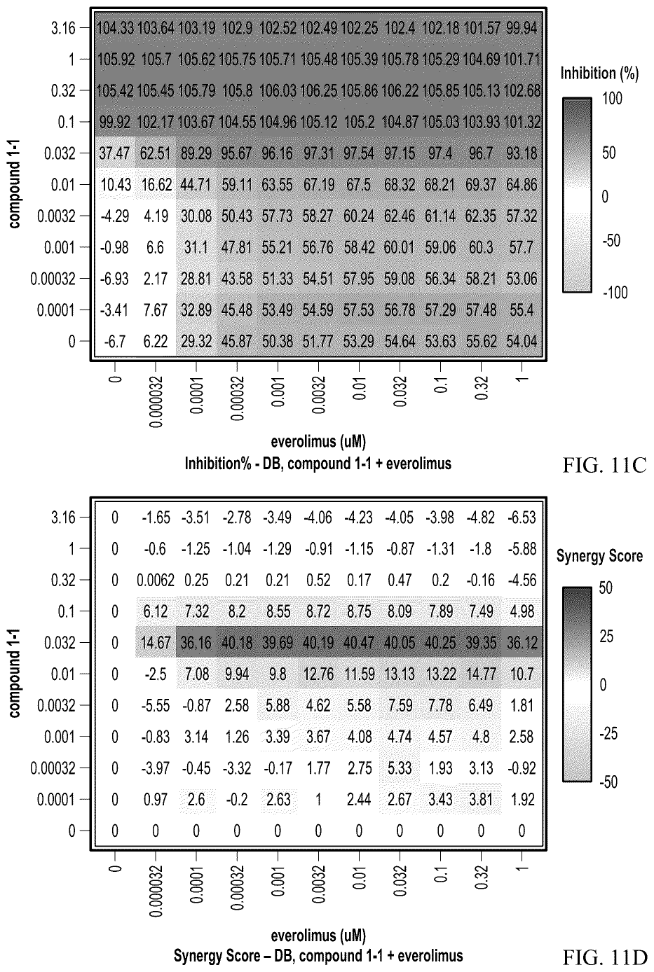

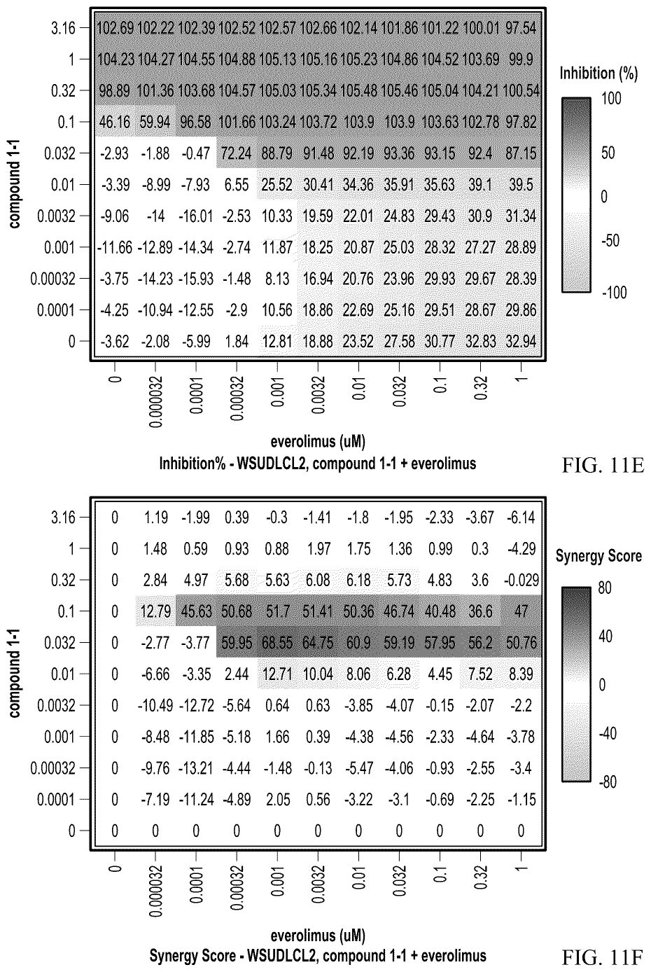

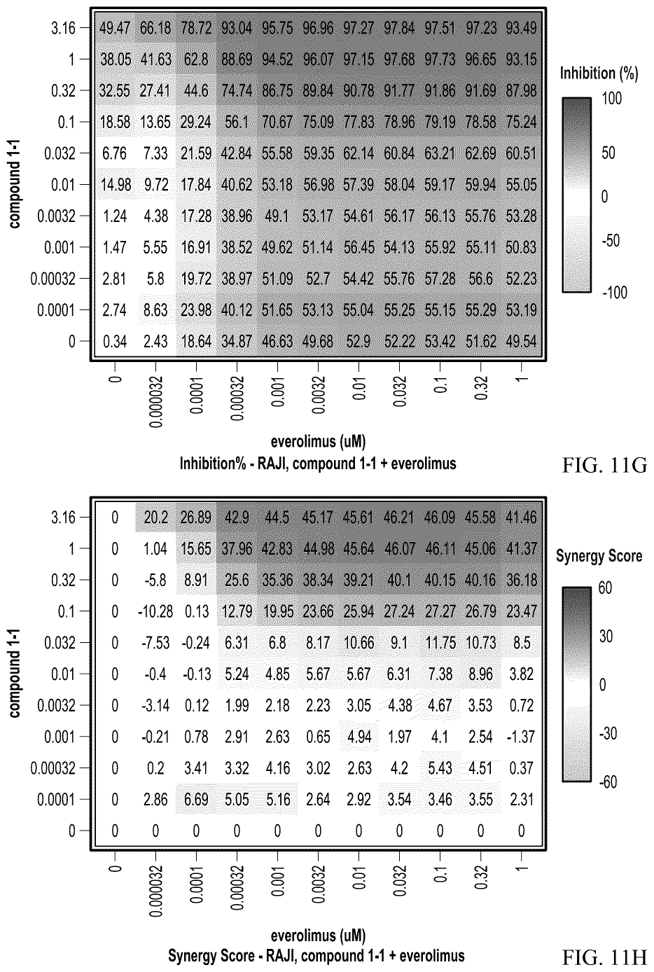

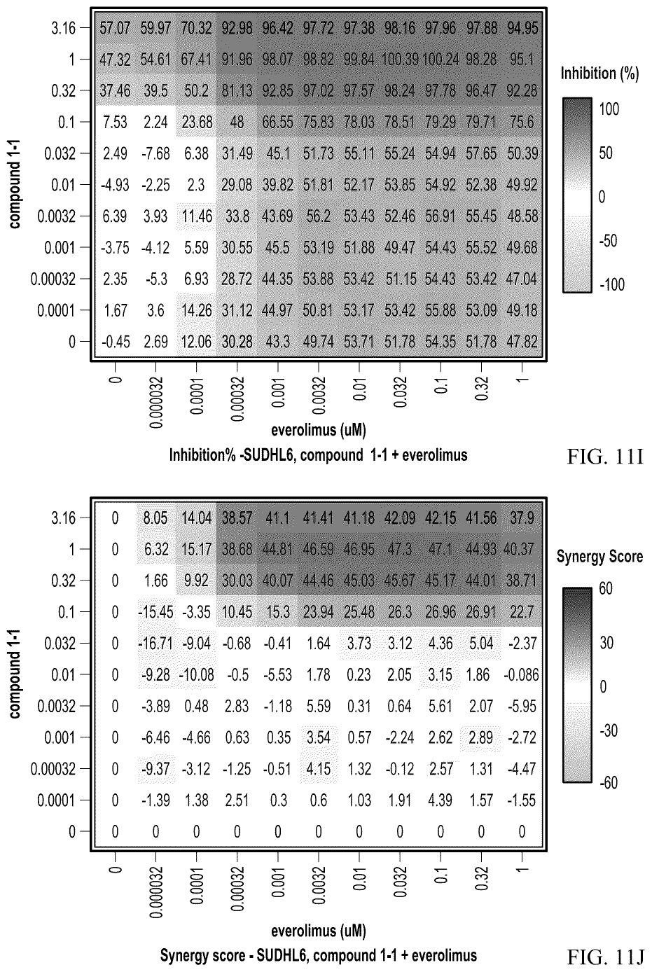

As in the lung cancer cell lines, the highest synergy scores were observed when combining compound 1-1 with the mTOR inhibitor everolimus (Table 8B). Again, high synergy scores for this combination were only detected in a relatively narrow range of concentrations of compound 1-1 for some but not all of the tested lymphoma cell lines, which largely depended on the activity of compound 1-1 as single agent (FIG. 11A-11J).

### Example 9. In Vitro Study of Compound 1-1 Combined with PI3K/Akt/mTOR Inhibitors in Multiple Myeloma (MM) Cell Lines

### Methods:

The study was conducted by Shanghai ChemPartner Co., Ltd (Study Number CPB-P21-25329). Details on the 5 multiple myeloma cell lines that were part of the experiment are listed in Table 9A.

TABLE 9A
Information on the 5 multiple myeloma cell lines used in
the study. Seeding density indicates the number of cells
seeded per well on the day before compound addition.

| Cell line | Supplier | Catalog number | Seeding density |
|---|---|---|---|
| MM.1S | ATCC | CRL-2974 | 5000 |
| OPM-2 | DSMZ | ACC 50 | 2500 |
| U266B1 | ATCC | TIB-196 | 5000 |
| RPMI 8226 | ATCC | CCL-155 | 2000 |
| KMS-34 | JCRB | JCRB1195 | 1500 |

Combination experiments were performed as described in Example 6.

### Results:

In vitro concentration matrix experiments were conducted to test whether combinations of the GSPT1 molecular glue degrader compound 1-1 and agents targeting the PI3K/AKT/mTOR pathway can synergistically reduce viability of multiple myeloma cells. The full set of data is summarized in Table 9B, where the mean Loewe synergy scores are presented.

TABLE 9B
Mean Loewe synergy scores of compound 1-1 combined with
PI3K/Akt/mTOR inhibitors in multiple myeloma cell lines

| | PI3K/Akt/ | MM cell lines | | | | |
|---|---|---|---|---|---|---|
| | mTOR | | | | RPMI | |
| target | inhibitor | MM.1S | OPM-2 | U266B1 | 8226 | KMS-34 |
| mTOR | everolimus | 6.7 | 5.3 | 4.16 | 20.75 | 16.19 |
| AKT | capivasertib | 2.63 | −1 | 1.76 | 4.81 | 7.48 |
| Pan- | buparlisib | 1.48 | −1.38 | −0.19 | 3.57 | 5.94 |
| PI3K | | | | | | |
| PI3K | alpelisib | 3.44 | −2.51 | 0.13 | 0.9 | 7.85 |
| alpha | | | | | | |

As before, the highest synergy scores were observed when combining compound 1-1 with the mTOR inhibitor everolimus (Table 9B), but additional less pronounced synergies were noted, for example between compound 1-1- and capiversertib, buparlisib, or alpelisib in the cell line KMS-34.

### Example 10. In Vivo Study of Compound 1-1 Combined with Everolimus in Estrogen Receptor-Positive Breast Cancer MCF7 Cell-Derived Xenografts (CDX)

### Methods:

Animals: BALB/c nude or SCID Beige mice, female, 6-8 weeks, weighing approximately 18-22 g, were purchased from certified vendors.

Tumor Inoculation for MCF-7 CDX model: Each mouse was inoculated subcutaneously at the right flank with MCF-7 tumor cells (10×10⁶) in 0.2 mL of PBS mixed with Matrigel (50:50) for tumor development. 170-Estradiol (0.36 mg) pellets (Innovative Research of America Cat. No.: SE-121, pellet size: 3.0 mm) were implanted 2-3 days before cell inoculation.

Mice were randomized and treatment was started when the average tumor volume reached approximately 150-200 mm³. The test article administration and the animal numbers in each group are shown in Table 10A. Compound 1-1 was formulated every day freshly as a solution in 5% DMSO/95% (30% w/v HPβCD in water). This formulation was also used as vehicle control. Everolimus was formulated as a suspension in 1% MC (400 cps), which was prepared every 3 days.

Tumor sizes were measured twice per week in two dimensions using a caliper, and the volume will be expressed in mm3 using the formula: V=0.5 a×b² where a and b are the long and short diameters of the tumor, respectively. Body weights were measured at the same time.

TABLE 10A
In vivo study design. (p.o., per os (oral). qd, quaque die (once daily))

| | | | | dose | dosing volume | dosing | |
|---|---|---|---|---|---|---|---|
| CDX | group | n | treatment | (mg/kg) | (μL/g) | route | schedule |
| MCF7 | 1 | 4 | vehicle | — | 10 | p.o. | qd for 28 days |
| MCF7 | 2 | 4 | everolimus | 2 | 10 | p.o. | qd for 28 days |
| MCF7 | 3 | 4 | compound | 10 | 10 | p.o. | qd for 28 days |
| | | | 1-1 | | | | |
| MCF7 | 4 | 4 | combination | 2 + 10 | 10 + 10 | p.o. | qd for 28 days |

### Results:

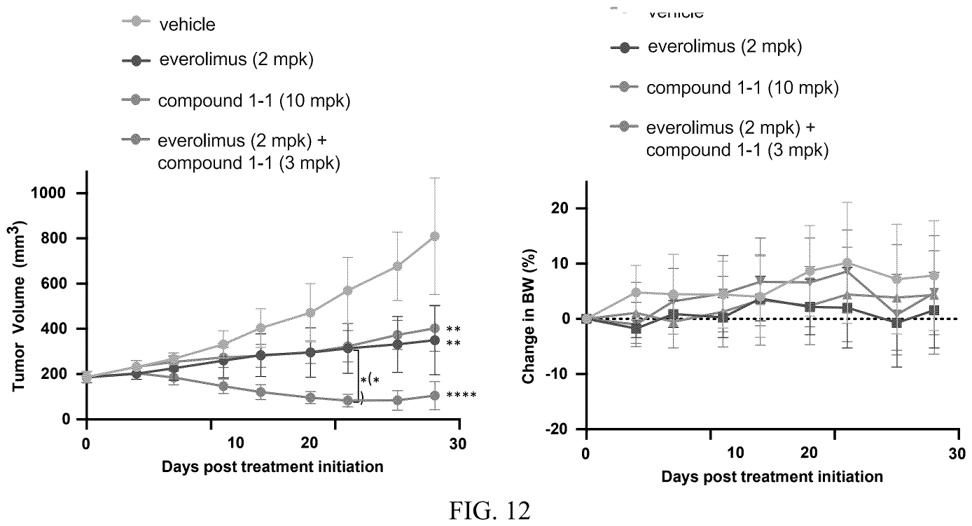

BALB/c nude mice bearing MCF7 xenografts were treated orally once a day with everolimus, compound 1-1, or a combination thereof. Vehicle controls consisted of animals receiving a daily oral administration of 5% DMSO/95% (30% w/v HPβCD in water). Details are listed in Table 10A and results for tumor volume and body weight over time are shown in FIG. 12. When administered as single agents, both everolimus and compound 1-1 produced a statistically significant antitumor effects (p<0.01, ANOVA with post hoc Tukey's test at day 28), albeit without inducing tumor regression. The combination of both agents led to tumor regression, which was highly significant (p<0.0001 vs vehicle). The combination-treated tumor volumes were not significantly different from tumor volumes in the single agent groups on day 28 (everolimus vs combination, p=0.13; compound 1-1 vs combination, p=0.06). However, these differences were significant on day 21, where variability of tumor volumes was lower (FIG. 12).

An approximation of drug interactions was made using the method described by Clarke (Clarke et al. 1997. Breast Cancer Res Treat. 46(2-3):255-78). This was applied to A TV and can estimate interactions from limited data. In short, for compound A, B, or the combination AB (with control group C), antagonism is predicted when (AB)/C>(A/C)×(B/C), an additive effect is predicted when (AB)/C=(A/C)×(B/C), and synergistic interaction is predicted to occur when (AB)/C<(A/C)×(B/C). Calculations in Table 10B indicate that the interaction of everolimus and compound 1-1 in the MCF7 CDX model is synergistic.

TABLE 10B
Assessment of synergy between everolimus and compound
1-1 in the MCF7 CDX model on day 28 by the Clarke method.
Related to tumor volumes displayed in FIG. 12. Average
values from four tumors per group are listed. Δ
TV, delta tumor volume. C = vehicle group, A =
everolimus-treated group, B = compound 1-1-treated
group, AB = combination-treated group.

| | | | | | | | (A/C) × | |
|---|---|---|---|---|---|---|---|---|
| | C | A | B | AB | A/C | B/C | (B/C) | (AB)/C |
| Δ TV | 623 | 164 | 216 | −82 | 0.26 | 0.35 | 0.09 | −0.13 |

No significant changes in body weight of the mice were noted on day 28.

### Example 11. In Vivo Study of Compound 1-1 Combined with Alpelisib in Treating Estrogen Receptor-Positive Breast Cancer in MCF7 Cell-Derived Xenografts (CDX)

### Methods:

Animals: BALB/c nude or SCID Beige mice, female, 6-8 weeks, weighing approximately 18-22 g, were purchased from certified vendors.

Tumor Inoculation for MCF-7 CDX model: Each mouse was inoculated subcutaneously at the right flank with MCF-7 tumor cells (10×10⁶) in 0.2 mL of PBS mixed with Matrigel (50:50) for tumor development. 17β-Estradiol (0.36 mg) pellets (Innovative Research of America Cat. No.: SE-121, pellet size: 3.0 mm) were implanted 2-3 days before cell inoculation.

Mice were randomized and treatment was started when the average tumor volume reached approximately 150-200 mm³. The test article administration and the animal numbers in each group are shown in Table 11A. Compound 1-1 was formulated every day freshly as a solution in 5% DMSO/95% (30% w/v HPβCD in water). This formulation was also used as vehicle control. Alpelisib was formulated as a solution in 20% captisol+80% water (pH adjusted to 4), which was prepared every week.

Tumor sizes were measured twice per week in two dimensions using a caliper, and the volume was expressed in mm³ using the formula: V=0.5 a×b² where a and b are the long and short diameters of the tumor, respectively. Body weights were measured at the same time.

TABLE 11A
In vivo study design. (p.o., per os (oral). qd, quaque die (once daily))

| | | | | dose | dosing volume | dosing | |
|---|---|---|---|---|---|---|---|
| CDX | group | n | treatment | (mg/kg) | (μL/g) | route | schedule |
| MCF7 | 1 | 4 | vehicle | — | 10 | p.o. | qd for 28 days |
| MCF7 | 2 | 4 | alpelisib | 50 | 10 | p.o. | qd for 28 days |
| MCF7 | 3 | 4 | compound 1- | 10 | 10 | p.o. | qd for 28 days |
| | | | 1 | | | | |
| MCF7 | 4 | 4 | combination | 50 + 10 | 10 + 10 | p.o. | qd for 28 days |

### Results:

BALB/c nude mice bearing MCF7 xenografts were treated orally once a day with alpelisib, compound 1-1, or a combination thereof. Vehicle controls consisted of animals receiving a daily oral administration of 5% DMSO/95% (30% w/v HPβCD in water). Details are listed in Table 11A and results for tumor volume and body weight over time are shown in FIG. 13. All treatments produced a significant anti-tumor effect compared to vehicle (p<0.001 for alpelisib, p<0.01 for compound 1-1, and p<0.0001 for combination, ANOVA with post hoc Tukey's test at day 28). Tumor volumes in the compound 1-1 treated arm were also significantly different from combination (p<0.05).

An approximation of drug interactions was again made using the method described by Clarke (Clarke 1997) as described above. Calculations in Table 11B indicate that the interaction of alpelisib and compound 1-1 in the MCF7 CDX model is synergistic.

TABLE 11B
Assessment of synergy between alpelisib and compound
1-1 in the MCF7 CDX model on day 28 by the Clarke method.
Related to tumor volumes displayed in FIG. 13. Average
values from four tumors per group are listed. Δ
TV, delta tumor volume. C = vehicle group, A =
alpelisib-treated group, B = compound 1-1-treated
group, AB = combination-treated group.

| | | | | | | | (A/C) × | |
|---|---|---|---|---|---|---|---|---|
| | C | A | B | AB | A/C | B/C | (B/C) | (AB)/C |
| Δ TV | 623 | 91 | 216 | −84 | 0.15 | 0.35 | 0.05 | −0.13 |

No significant changes in body weight of the mice were noted on day 28.

### Example 12. In Vivo Study of Compound 1-1 Combined with Everolimus in Triple Negative Breast Cancer CAL51 Cell-Derived Xenografts (CDX)

### Methods:

Animals: BALB/c nude or SCID Beige mice, female, 6-8 weeks, weighing approximately 18-22 g, were purchased from certified vendors.

Tumor Inoculation for CAL-51 CDX model: Each mouse was inoculated subcutaneously at the right flank with CAL51 cells (5*10⁶) cells in 0.2 mL of PBS mixed with Matrigel (50:50) for tumor development.

Mice were randomized and treatment was started when the average tumor volume reached approximately 150-200 mm3. The test article administration and the animal numbers in each group are shown in Table 12A. Compound 1-1 was formulated every day freshly as a solution in 5% DMSO/95% (30% w/v HPβCD in water). This formulation was also used as vehicle control. Everolimus was formulated as a suspension in 1% MC (400 cps), which was prepared every 3 days.

Tumor sizes were measured twice per week in two dimensions using a caliper, and the volume will be expressed in mm3 using the formula: V=0.5 a×b2 where a and b are the long and short diameters of the tumor, respectively. Body weights were measured at the same time.

TABLE 12A
In vivo study design. (p.o., per os (oral). qd, quaque die (once daily))

| | | | | dose | dosing volume | dosing | |
|---|---|---|---|---|---|---|---|
| CDX | group | n | treatment | (mg/kg) | (μL/g) | route | schedule |
| CAL51 | 1 | 4 | vehicle | — | 10 | p.o. | qd for 21 days |
| CAL51 | 2 | 4 | everolimus | 2 | 10 | p.o. | qd for 21 days |
| CAL51 | 3 | 4 | compound | 3 | 10 | p.o. | qd for 21 days |
| | | | 1-1 | | | | |
| CAL51 | 4 | 4 | combination | 2 + 3 | 10 + 10 | p.o. | qd for 21 days |

### Results

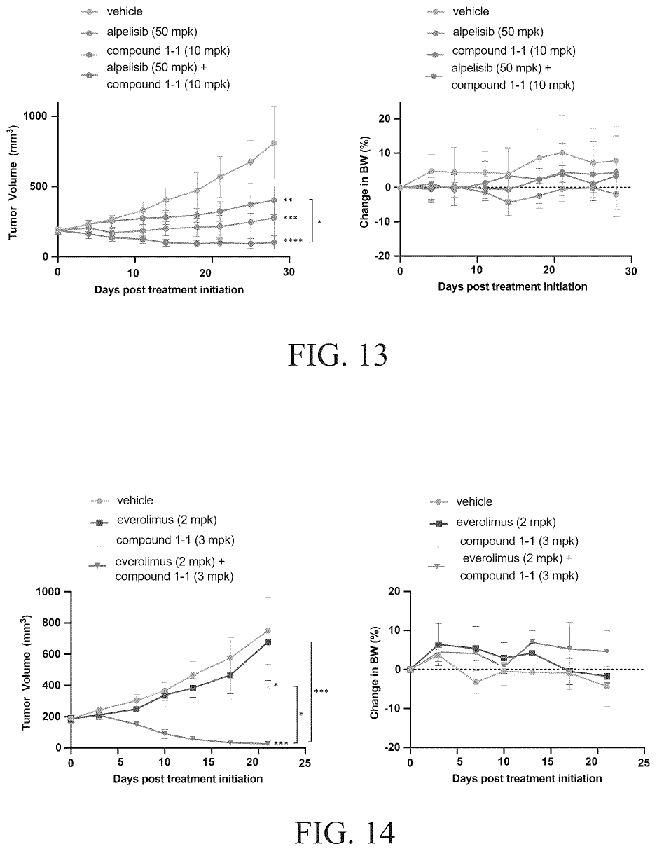

Female SCID Beige mice bearing CAL51 xenografts were treated orally once a day with everolimus, compound 1-1, or a combination thereof. Vehicle controls consisted of animals receiving a daily oral administration of 5% DMSO/95% (30% w/v HPβCD in water). Details are listed in Table 12B and results for tumor volume and body weight over time are shown in FIG. 14. When administered as single agent, compound 1-1 produced a statistically significant antitumor effect (p<0.05, ANOVA with post hoc Tukey's test at day 21), albeit without inducing tumor regression. Everolimus as single agent did not significantly impede tumor growth. In contrast, the combination of both agents led to marked tumor regression, which was highly significant (p<0.001 vs vehicle). The combination-treated tumor volumes were also significantly different from tumor volumes in the everolimus (p<0.001) or compound 1-1 (p<0.05) single agent groups.

An approximation of drug interactions was made using the method described by Clarke (Clarke 1997) as described above. Calculations in Table 12B indicate that the interaction of everolimus and compound 1-1 in the CAL51 CDX model is synergistic.

TABLE 12B
Assessment of synergy between everolimus and compound 1-1 in
the CAL51 CDX model on day 21 by the Clarke method. Related
to tumor volumes displayed in FIG. 14. Average values from
four tumors per group are listed. Δ TV, delta tumor volume.
C = vehicle group, A = everolimus-treated group,
B = compound 1-1-treated group, AB = combination-treated group.

| | | | | | | | (A/C) × | |
|---|---|---|---|---|---|---|---|---|
| | C | A | B | AB | A/C | B/C | (B/C) | (AB)/C |
| Δ TV | 562 | 491 | 215 | −162 | 0.87 | 0.38 | 0.33 | −0.29 |

No significant changes in body weight of the mice were noted on day 21.

### Example 13. In Vivo Study of Compound 1-1 Combined with Alpelisib in Triple Negative Breast Cancer in CAL51 Cell-Derived Xenografts (CDX)

### Methods:

Animals: BALB/c nude or SCID Beige mice, female, 6-8 weeks, weighing approximately 18-22 g, were purchased from certified vendors.

Tumor Inoculation for CAL-51 CDX model: Each mouse was inoculated subcutaneously at the right flank with CAL51 cells (5*10⁶) cells in 0.2 mL of PBS mixed with Matrigel (50:50) for tumor development.

Mice were randomized and treatment was started when the average tumor volume reached approximately 150-200 mm³. The test article administration and the animal numbers in each group are shown in Table 13A. Compound 1-1 was formulated every day freshly as a solution in 5% DMSO/95% (30% w/v HPβCD in water). This formulation was also used as vehicle control. Alpelisib was formulated as a solution in 20% captisol+80% water (pH adjusted to 4), which was prepared every week.

Tumor sizes were measured twice per week in two dimensions using a caliper, and the volume was expressed in mm3 using the formula: V=0.5 a×b2 where a and b are the long and short diameters of the tumor, respectively. Body weights were measured at the same time.

TABLE 13A
In vivo study design. (p.o., per os (oral). qd, quaque die (once daily))

| | | | | dose | dosing volume | dosing | |
|---|---|---|---|---|---|---|---|
| CDX | group | n | treatment | (mg/kg) | (μL/g) | route | schedule |
| CAL51 | 1 | 4 | vehicle | — | 10 | p.o. | qd for 21 days |
| CAL51 | 2 | 4 | alpelisib | 20 | 10 | p.o. | qd for 21 days |
| CAL51 | 3 | 4 | compound 1-1 | 3 | 10 | p.o. | qd for 21 days |
| CAL51 | 4 | 4 | combination | 20 + 3 | 10 + 10 | p.o. | qd for 21 days |

### Results

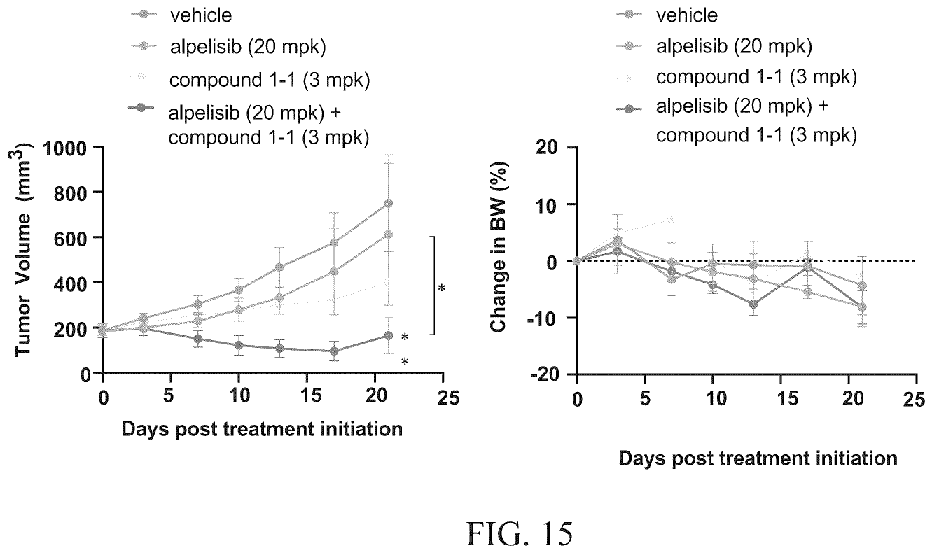

Female SCID Beige mice bearing CAL51 xenografts were treated orally once a day with alpelisib, compound 1-1, or a combination thereof. Vehicle controls consisted of animals receiving a daily oral administration of 5% DMSO/95% (30% w/v HPβCD in water). Details are listed in Table 13A and results for tumor volume and body weight over time are shown in FIG. 15. When administered as single agent, neither alpelisib nor compound 1-1 produced a statistically significant antitumor effect, whereas the combination did (p<0.01 compared to vehicle, ANOVA with post hoc Tukey's test at day 21). Tumor volumes in the alpelisib single agent arm were also significantly different from combination (p<0.05).

An approximation of drug interactions was made using the method described by Clarke (Clarke 1997) as described above. Calculations in Table 13B indicate that the interaction of alpelisib and compound 1-1 in the CAL51 CDX model is synergistic.

TABLE 13B
Assessment of synergy between alpelisib and compound
1-1 in the CAL51 CDX model on day 21 by the Clarke method.
Related to tumor volumes displayed in FIG. 15. Average
values from four tumors per group are listed. Δ
TV, delta tumor volume. C = vehicle group, A =
alpelisib-treated group, B = compound 1-1-treated
group, AB = combination-treated group.

| | | | | | | | (A/C) × | |
|---|---|---|---|---|---|---|---|---|
| | C | A | B | AB | A/C | B/C | (B/C) | (AB)/C |
| Δ TV | 562 | 425 | 215 | −22 | 0.76 | 0.38 | 0.29 | −0.04 |

No significant changes in body weight of the mice were noted on day 21.

### Example 14. In Vivo Study of Compound 1-1 Combined with Everolimus in Non-Small Cell Lung Cancer NCI-H1155 Cell-Derived Xenografts (CDX)

### Methods:

Animals: BALB/c nude mice, female, 6-8 weeks, weighing approximately 18-24 g, were purchased from certified vendors.

Tumor Inoculation for NCI-H1155 CDX model: NCI-H1155 cells were obtained from ATCC and cultured in vitro according to the supplier's instructions. Each mouse was inoculated subcutaneously at the right flank with NCI-H1155 cells (5*10⁵) cells in 0.2 ml mixture of cell culture medium with Matrigel (medium: Matrigel=1:1) for tumor development.

Mice were randomized and treatment was started when the average tumor volume reached approximately 200 (range approximately 100-350) mm³. The test article administration and the animal numbers in each group are shown in Table 14. The maximal duration of test article administration was 43 days (groups 5 and 15), but most groups were terminated earlier, either because all tumors had exceeded the maximal tumor volume of 2000 mm³ (groups 1, 2, 4, 6, 7), or a few days after all mice had progressed according to our definition for Kaplan-Meier analysis (groups 3, 8, 9, 10, 11, 12, 13, 14). Compound 1-1 powder, produced as a 20% spray-dried dispersion, was formulated every day freshly as a suspension in vehicle 1: 0.5% (weight per volume) methylcellulose (400 cps) in water. Everolimus was formulated every day freshly as a solution in vehicle 2: 30% propylene glycol, 5% Tween-80 and 65% water. Both compounds were administered orally.

Tumor sizes were measured twice per week in two dimensions using a caliper, and the volume was expressed in mm³ using the formula: V=0.5 a×b² where a and b are the long and short diameters of the tumor, respectively. Body weights were measured at the same time.

TABLE 14
In vivo study design for NCI-H1155 (example 15) and
NCI-H1770 (example 15) CDX models. (p.o., per os
(oral). qd, quaque die (once daily). 5 on/9 off,
5 daily doses followed by 9 days without dosing)

| | | | | dosing | | |
|---|---|---|---|---|---|---|
| | | | dose | volume | dosing | |
| group | n | treatment | (mg/kg) | (μL/g) | route | schedule |
| 1 | 5 | vehicle 1 + | — | 10 + 10 | p.o. | qd + qd |
| | | vehicle 2 | | | | |
| 2 | 5 | compound | 1 | 10 + 10 | p.o. | qd + qd |
| | | 1-1 + vehicle 2 | | | | |
| 3 | 5 | compound | 3 | 10 + 10 | p.o. | qd + qd |
| | | 1-1 + vehicle 2 | | | | |
| 4 | 5 | compound | 3 | 10 + 10 | p.o. | 5 on/9 |
| | | 1-1 + vehicle 2 | | | | off + qd |
| 5 | 5 | compound | 10 | 10 + 10 | p.o. | 5 on/9 |
| | | 1-1 + vehicle 2 | | | | off + qd |
| 6 | 5 | vehicle 1 + | 2 | 10 + 10 | p.o. | qd + qd |
| | | everolimus | | | | |
| 7 | 5 | vehicle 1 + | 5 | 10 + 10 | p.o. | qd + qd |
| | | everolimus | | | | |
| 8 | 5 | compound 1-1 + | 1 + 2 | 10 + 10 | p.o. | qd + qd |
| | | everolimus | | | | |
| 9 | 5 | compound 1-1 + | 3 + 2 | 10 + 10 | p.o. | qd + qd |
| | | everolimus | | | | |
| 10 | 5 | compound 1-1 + | 3 + 2 | 10 + 10 | p.o. | 5 on/9 |
| | | everolimus | | | | off + qd |
| 11 | 5 | compound 1-1 + | 10 + 2 | 10 + 10 | p.o. | 5 on/9 |
| | | everolimus | | | | off + qd |
| 12 | 5 | compound 1-1 + | 1 + 5 | 10 + 10 | p.o. | qd + qd |
| | | everolimus | | | | |
| 13 | 5 | compound 1-1 + | 3 + 5 | 10 + 10 | p.o. | qd + qd |
| | | everolimus | | | | |
| 14 | 5 | compound 1-1 + | 3 + 5 | 10 + 10 | p.o. | 5 on/9 |
| | | everolimus | | | | off + qd |
| 15 | 5 | compound 1-1 + | 10 + 5 | 10 + 10 | p.o. | 5 on/9 |
| | | everolimus | | | | off + qd |

### Results

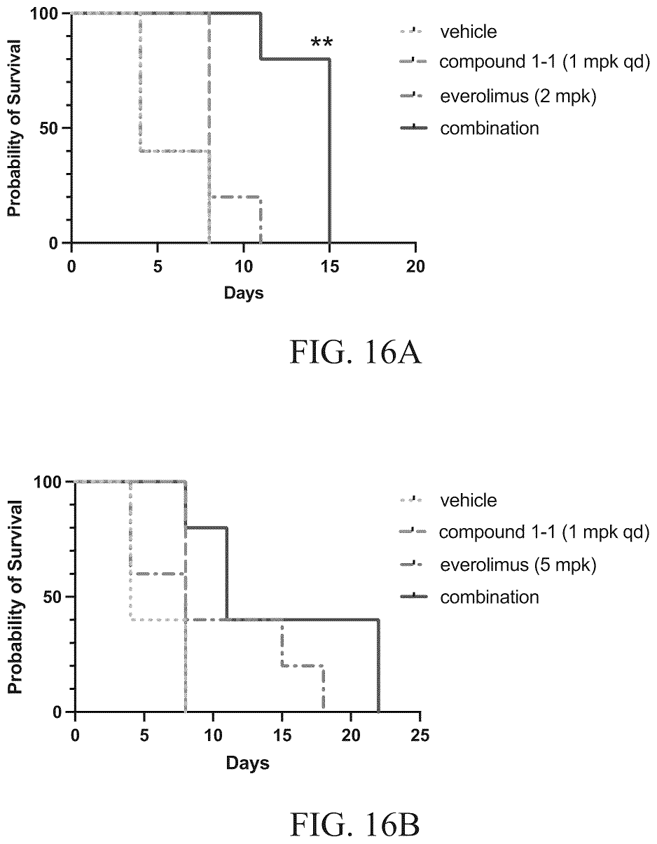

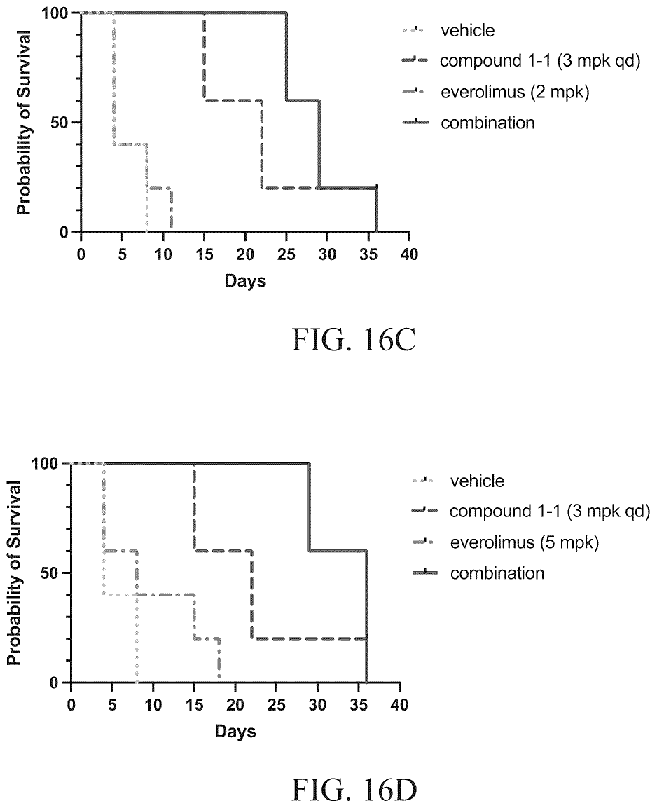

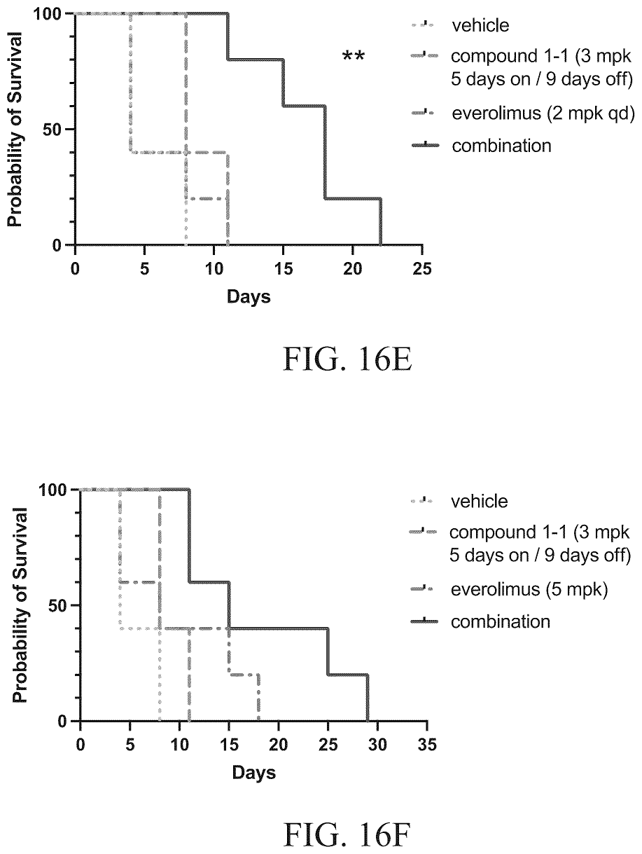

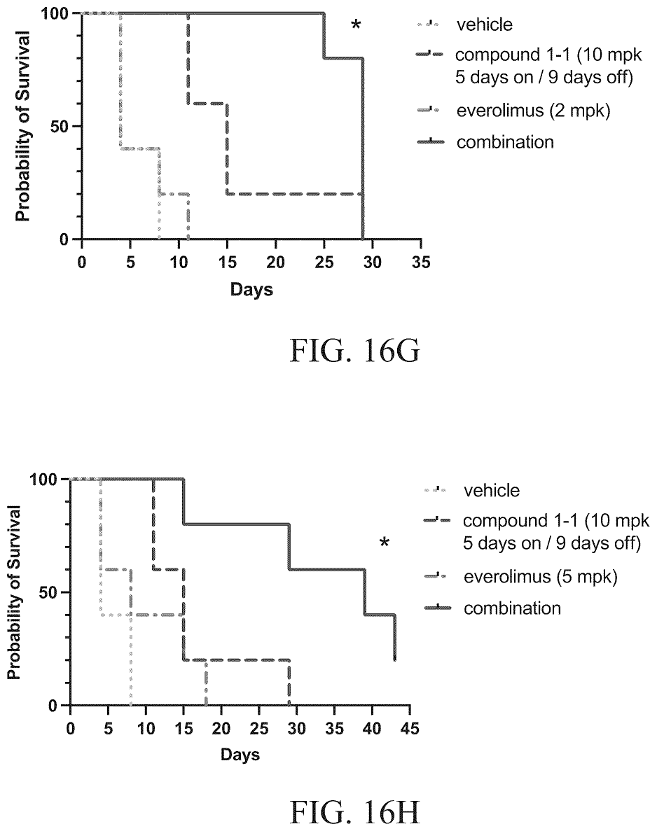

Female BALB/c nude mice bearing NCI-H1155 neuroendocrine lung cancer xenografts were treated orally with vehicle controls, compound 1-1, everolimus, or combinations of compound 1-1 and everolimus as indicated in Table 14. Results for anti-tumor efficacy are shown in FIG. 16. Given the rapid and heterogenous growth of the NCI-H1155 model, individual tumors in groups treated with less efficacious regimens quickly exceeded the maximal volume of 2000 mm3 that was defined as a termination criterion for animal welfare reasons. As a consequence, group sizes became unequal over time, precluding a meaningful representation of the anti-tumor efficacy results by displaying tumor volumes. (Drop-out of single tumors due to exceeding the maximal volume leads to a selection of better responders over time, therefore the average tumor volume as a measure of anti-tumor efficacy becomes misleading.) To mitigate this analytical challenge, we chose to display anti-tumor efficacy as Kaplan-Meier (Probability of Survival) curves, where survival was arbitrarily defined as tumor volume 800 mm³. Therefore, each drop of the curve by 20% represents one mouse in which tumor volume reached or exceeded 800 mm³. We believe that this way of displaying the data makes groups more comparable.

Each of the 8 graphs in FIG. 16A-FIG. 16H displays vehicle control, one single agent dose regimen each of compound 1-1 and everolimus, and the respective combination. Vehicle and single agent groups are repeated across graphs in order to display results in a systematic manner. While longer progression-free survival of the combination group is apparent in all graphs, the advantage was not always statistically significant. We used log-rank (Mantel-Cox) tests to compare the combination treatment group to the more efficacious of the two respective single agent groups. Statistically significant superiority of the combination regimen is symbolized by asterisks.

Body weight loss was observed in a minority of mice with some apparent enrichment in the combination groups but did not require intervention because it remained below 15% in all cases.

### Example 15. In Vivo Study of Compound 1-1 Combined with Everolimus in Neuroendocrine Non-Small Cell Lung Cancer NCI-111770 Cell-Derived Xenografts (CDX)

### Methods:

Animals: BALB/c nude mice, female, 6-8 weeks, weighing approximately 18-22 g, were purchased from certified vendors.

Tumor Inoculation for NCI-H1770 CDX model: NCI-H1770 cells were obtained from ATCC and cultured in vitro according to the supplier's instructions. Each mouse was inoculated subcutaneously at the right flank with NCI-H1770 cells (5*10⁷) cells in 0.2 ml mixture of cell culture medium with Matrigel (medium: Matrigel=1:1) for tumor development.

Mice were randomized and treatment was started when the average tumor volume reached approximately 160 (range approximately 90 to 280) mm³. The test article administration and the animal numbers in each group are shown in Table 14. The maximal duration of test article administration was 49 days (groups 3, 4, 5, 7, 9, 11, 13, 14, 15). A subset of groups was terminated after 40 days (groups 1, 2, 6, 8, 12) after all or most tumors had progressed according to our definition for Kaplan-Meier analysis. Compound 1-1 powder, produced as a 20% spray-dried dispersion, was formulated every day freshly as a suspension in vehicle 1: 0.5% (weight per volume) methylcellulose (400 cps) in water. Everolimus was formulated every day freshly as a solution in vehicle 2: 30% propylene glycol, 5% Tween-80 and 65% water. Both compounds were administered orally.

Tumor sizes were measured twice per week in two dimensions using a caliper, and the volume was expressed in mm³ using the formula: V=0.5 a×b² where a and b are the long and short diameters of the tumor, respectively. Body weights were measured at the same time.

### Results

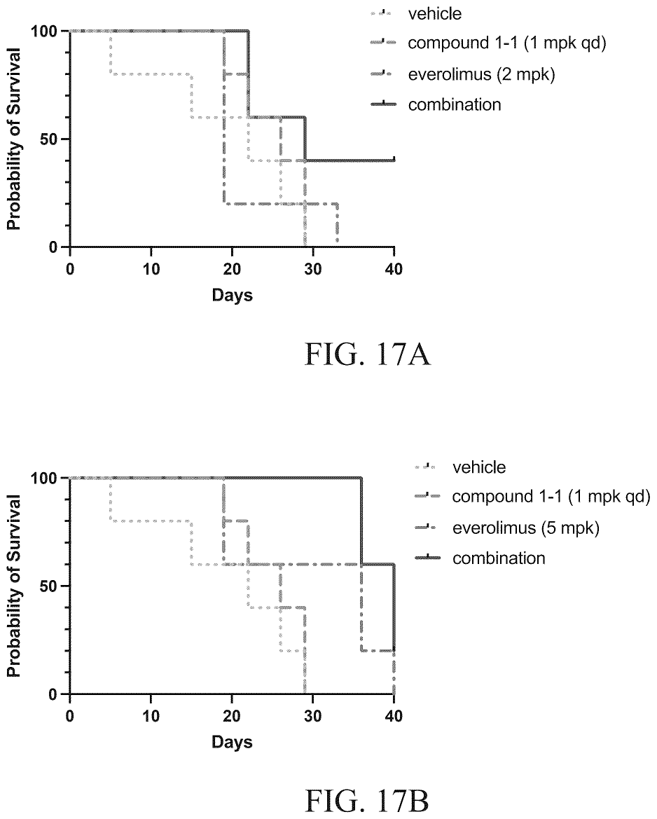

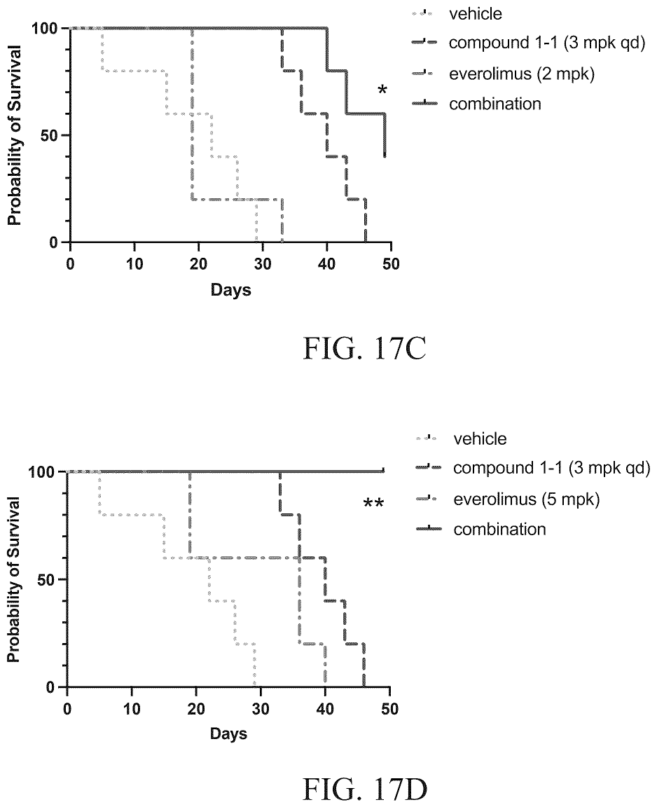

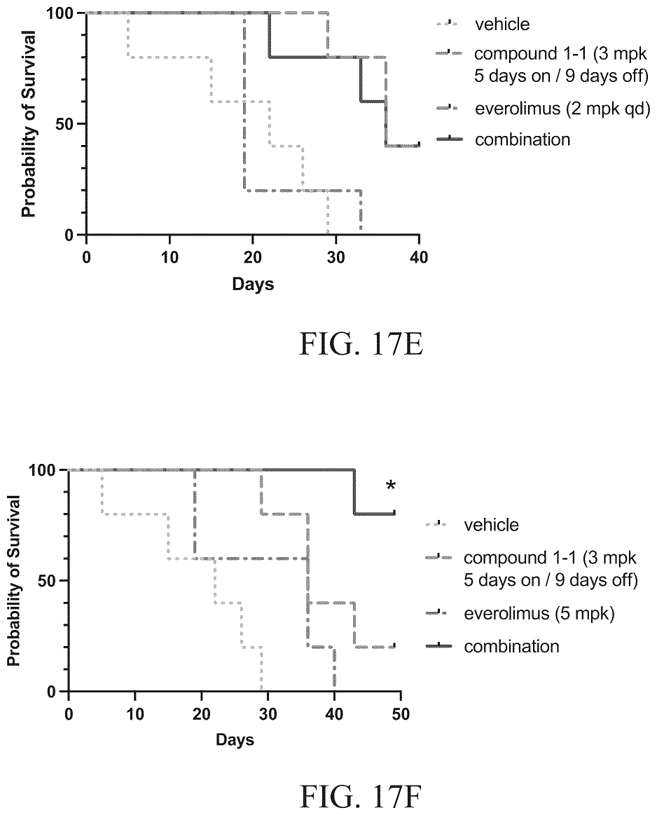

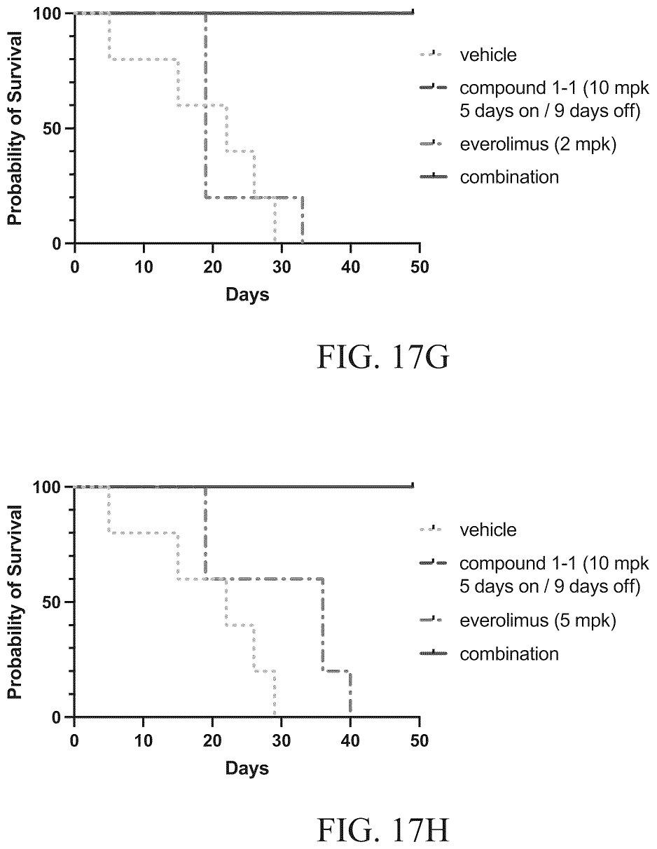

Female BALB/c nude mice bearing NCI-H1770 neuroendocrine lung cancer xenografts were treated orally with vehicle controls, compound 1-1, everolimus, or combinations of compound 1-1 and everolimus as indicated in Table 14. Results for anti-tumor efficacy are shown in FIG. 17. Anti-tumor efficacy is displayed as Kaplan-Meier (Probability of Survival) curves, where survival was arbitrarily defined as tumor volume 800 mm³, for the same reasons as explained in Example 14.

Each of the 8 graphs in FIG. 17A-FIG. 17H represents vehicle control, one dose regimen each of compound 1-1 and everolimus, and the respective combination. Vehicle and single agent groups are repeated across graphs in order to display results in a systematic manner. Longer progression-free survival of the combination group is apparent in a subset of the graphs. To assess statistical significance, we used log-rank (Mantel-Cox) tests to compare the combination treatment group to the more efficacious of the two respective single agent groups. Statistically significant superiority of the combination regimen is symbolized by asterisks.

Minor body weight loss was observed in a small subset of mice that did not require any intervention.

### Example 16. In Vivo Study of Compound 1-1 Combined with Everolimus in Six Different Patient-Derived Xenograft (PDX) Models of Lung Cancer

### Methods:

Models: The study was conducted at Crown Bioscience with the PDX models LU5188 (small cell lung neuroendocrine carcinoma), LU5247 (non-small cell lung adenocarcinoma), LU5236 (small cell lung neuroendocrine carcinoma), LU1508 (small cell lung neuroendocrine carcinoma), LU5215 (small cell lung neuroendocrine carcinoma), and LU5137 (large cell neuroendocrine carcinoma).
Animals: BALB/c nude (LU1508) or NOD/SCID (all other models) female mice, 5-7 weeks old, weighing approximately 22-25 g, were purchased from certified vendors.

Tumor Inoculation: Fresh tumor tissues was harvest from tumor bearing mice, cut into small pieces (approximately 2-3 mm in diameter) and was kept in RPMI1640 cell culture medium at 4° C.). Lidocaine cream was applied to the PDX fragment inoculation site 1 hour before the operation. In addition, hair was removed from NOD/SCID mice before fragment implantation. Each animal was disinfected at the inoculation site with alcohol swap, PDX tumor fragments were inserted subcutaneously at the upper right dorsal flank with a trocar. The trocar was withdrawn any air bubbles under the skin were extruded.

Mice were randomized and treatment was started when the average tumor volume reached approximately 100 (range 60-170) mm³. The test article administration and the animal numbers in each group are shown in Tables 15A, 15B and 15C. Compound 1-1 powder, produced as a 20% spray-dried dispersion, was formulated every day freshly as a suspension in vehicle 1: 0.5% (weight per volume) methylcellulose (400 cps) in water. Everolimus was formulated every day freshly as a solution in vehicle 2: 30% propylene glycol, 5% Tween-80 and 65% water. Both compounds were administered orally.

Tumor sizes were measured twice per week in two dimensions using a caliper, and the volume was expressed in mm³ using the formula: V=0.5 a×b² where a and b are the long and short diameters of the tumor, respectively. Body weights were measured at the same time.

TABLE 15A
In vivo study design for PDX model LU5188. (p.o., per
os (oral). qd, quaque die (once daily). 5 on/9 off,
5 daily doses followed by 9 days without dosing)

| | | | | dosing | | |
|---|---|---|---|---|---|---|
| | | | dose | volume | dosing | |
| group | n | treatment | (mg/kg) | (μL/g) | route | schedule |
| 1 | 6 | vehicle 1 | — | 10 | p.o. | qd |
| 2 | 6 | compound 1-1 | 3 | 10 | p.o. | qd |
| 3 | 6 | compound 1-1 | 10 | 10 | p.o. | 5 on/9 off |
| 4 | 6 | everolimus | 5 | 10 | p.o. | qd |
| 5 | 6 | compound 1-1 + | 3 + 5 | 10 + 10 | p.o. | qd + qd |
| | | everolimus | | | | |
| 6 | 6 | compound 1-1 + | 10 + 5 | 10 + 10 | p.o. | 5 on/9 |
| | | everolimus | | | | off + qd |

TABLE 15B
In vivo study design for PDX models LU5247, LU1508, LU5215 and
LU5137. (p.o., per os (oral). qd, quaque die (once daily). 5
on/9 off, 5 daily doses followed by 9 days without dosing)

| | | | | dosing | | |
|---|---|---|---|---|---|---|
| | | | dose | volume | dosing | |
| group | n | treatment | (mg/kg) | (μL/g) | route | schedule |
| 1 | 6 | vehicle 1 | — | 10 | p.o. | qd |
| 2 | 6 | compound 1-1 | 3 | 10 | p.o. | 5 on/9 off |
| 3 | 6 | compound 1-1 | 10 | 10 | p.o. | 5 on/9 off |
| 4 | 6 | everolimus | 5 | 10 | p.o. | qd |
| 5 | 6 | compound 1-1 + | 3 + 5 | 10 + 10 | p.o. | 5 on/9 |
| | | everolimus | | | | off + qd |
| 6 | 6 | compound 1-1 + | 10 + 5 | 10 + 10 | p.o. | 5 on/9 |
| | | everolimus | | | | off + qd |

TABLE 15C
In vivo study design for PDX model LU5236. (p.o., per
os (oral). qd, quaque die (once daily). 5 on/9 off,
5 daily doses followed by 9 days without dosing)

| | | | | dosing | | |
|---|---|---|---|---|---|---|
| | | | dose | volume | dosing | |
| group | n | treatment | (mg/kg) | (μL/g) | route | schedule |
| 1 | 6 | vehicle 1 | — | 10 | p.o. | qd |
| 2 | 6 | compound 1-1 | 3 | 10 | p.o. | 5 on/9 off |
| 3 | 6 | compound 1-1 | 10 | 10 | p.o. | 5 on/9 off |
| 4 | 6 | everolimus | 5 | 10 | p.o. | qd |
| 5 | 6 | compound 1-1 + | 3 + 5 | 10 + 10 | p.o. | 5 on/9 |
| | | everolimus | | | | off + qd |
| 6 | 6 | compound 1-1 + | 10 + 5 | 10 + 10 | p.o. | 5 on/9 |
| | | everolimus | | | | off + qd |

### Results

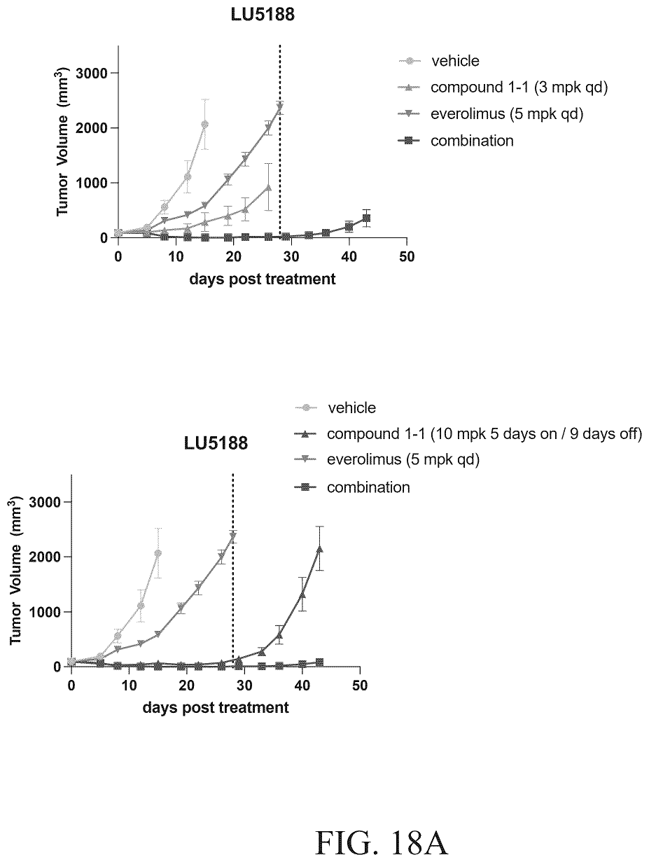

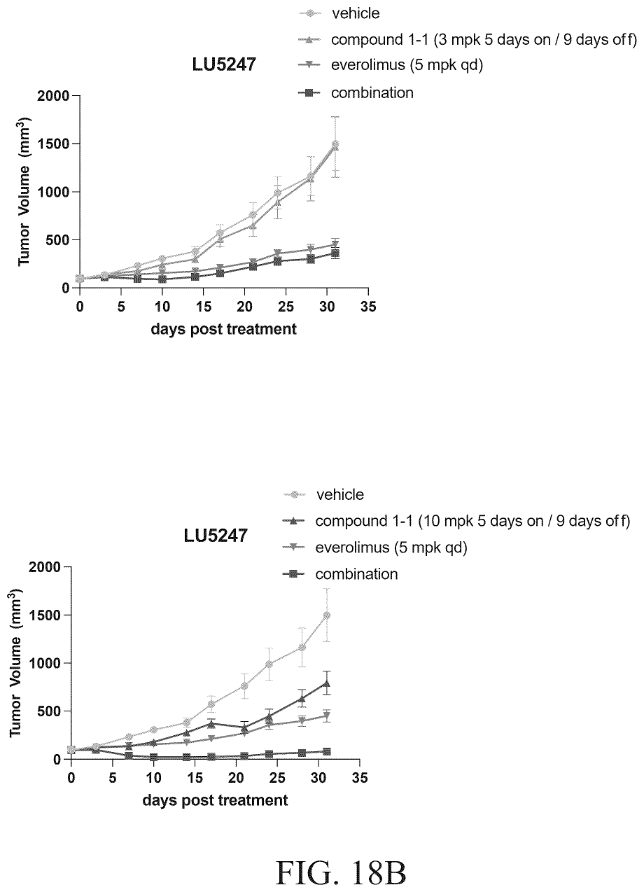

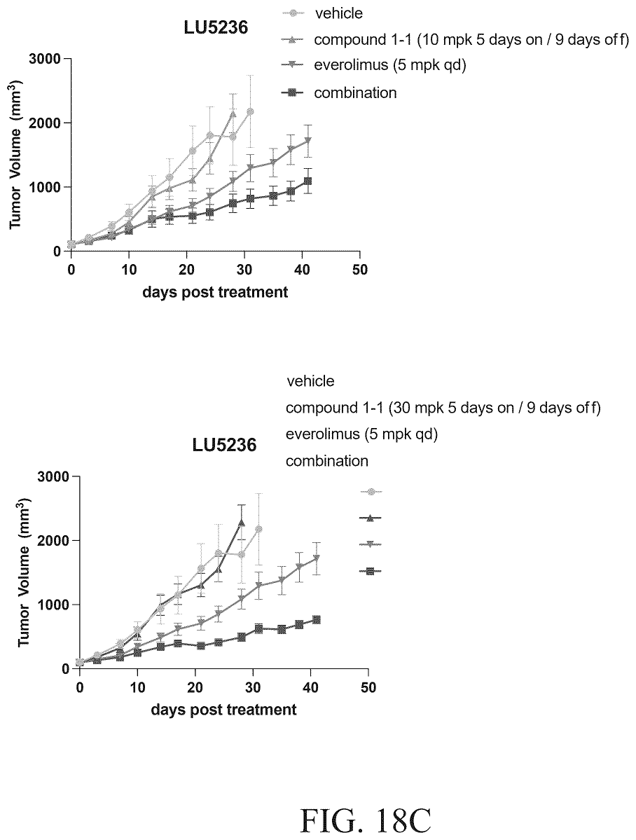

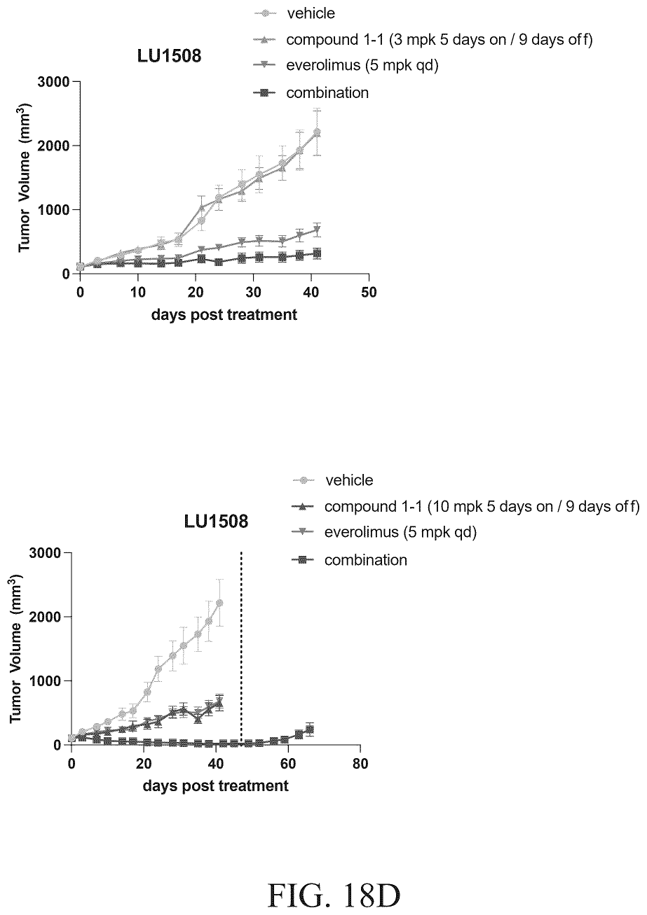

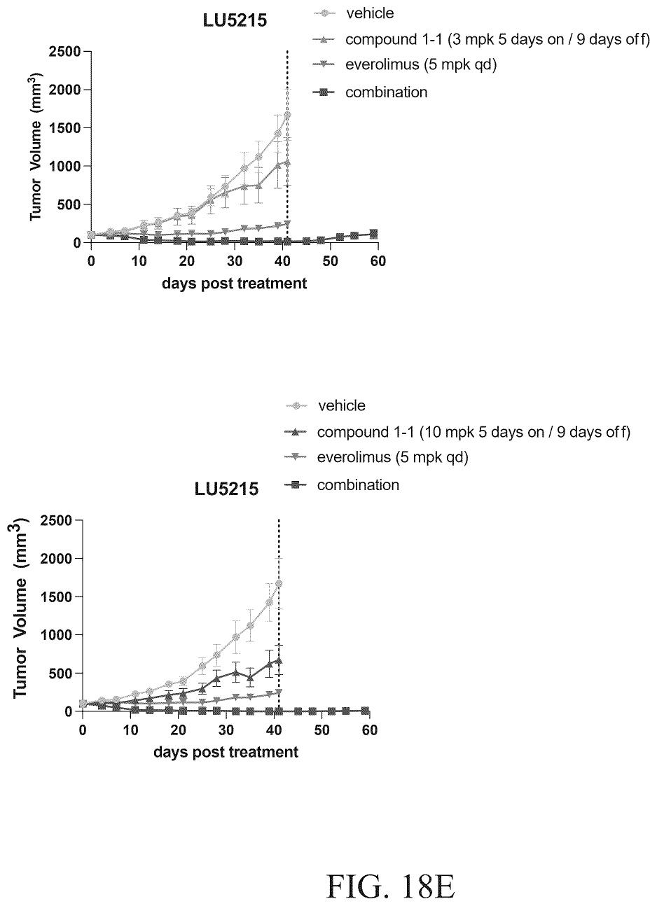

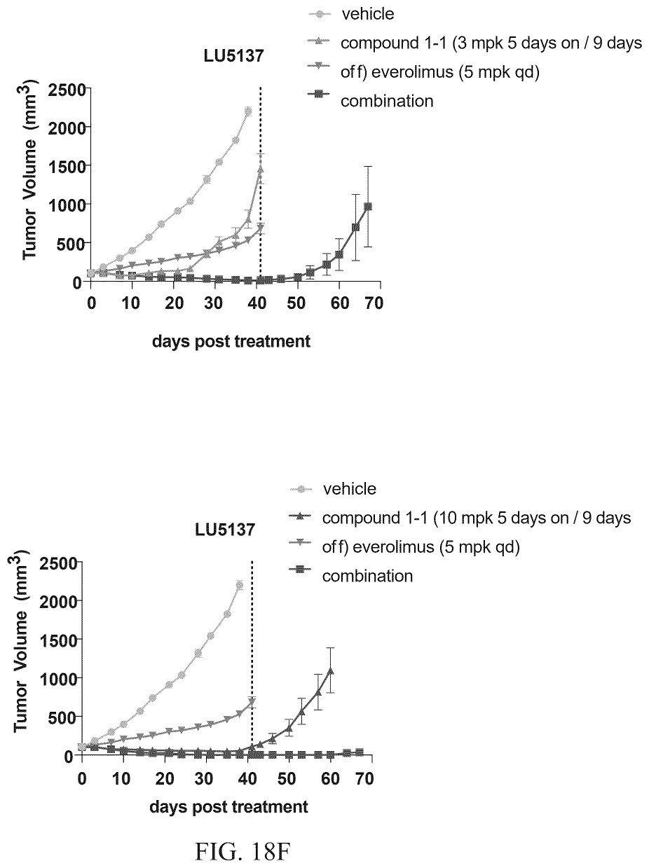

Six different PDX models grown in immunodeficient mice were treated with vehicle control, compound 1-1 at two different doses and/or schedules, everolimus at a dose of 5 mg/kg once daily, or combinations of the two compound 1-1 regimens with everolimus, respectively. Different compound 1-1 doses and schedules were applied to different PDX models as indicated in Tables 15A, 15B and 15C.
Tumor volumes over time are shown in FIG. 18A-FIG. 18F. For better readability, the data for each model is split into two graphs where the respective lower compound 1-1 dose and its combination are displayed in the upper graph and the higher dose in the lower. Vehicle and everolimus curves are thus repeated in the two graphs for each model. No predetermined study durations were applied, but instead treatment durations were adapted individually per group based on observed anti-tumor efficacy with the aim to either demonstrate a clear combination benefit, i.e. better anti-tumor efficacy of combination than either of the two constituting single agent regimens, or to terminate the study when there is a clear trend for absence of a combination benefit, i.e. anti-tumor efficacy of combination was similar to at least one of the constituting single agent regimens for a prolonged period. A combination benefit is apparent in the majority of displayed graphs in FIG. 18A-FIG. 18F. In some groups with very pronounced anti-tumor efficacy, the measurement of tumor volume was continued after cessation of treatment to assess the kinetics of re-growth. In these cases, the end of treatment is marked with a vertical dotted line.
Body weight loss was caused by everolimus dosing and in some of the models by the tumor itself (tumor-induced cachexia). It was mitigated individually for each mouse individually as needed by providing additional nutrition (nutrigel) and by drug holidays. With both these measures together, body weight loss of more than 20% was avoided in the vast majority of mice.

```
SEQUENCES
>PIK3CA [organism = Homo sapiens]
Sequence ID NO. 1
                10 20 30 40 50
MPPRPSSGEL WGIHLMPPRI LVECLLPNGM IVTLECLREA TLITIKHELF
                60 70 80 90 100
KEARKYPLHQ LLQDESSYIF VSVTQEAERE EFFDETRRLC DLRLFQPFLK
              110 120 130 140 150
VIEPVGNREE KILNREIGFA IGMPVCEFDM VKDPEVQDFR RNILNVCKEA
              160 170 180 190 200
VDLRDLNSPH SRAMYVYPPN VESSPELPKH IYNKLDKGQI IVVIWVIVSP
              210 220 230 240 250
NNDKQKYTLK INHDCVPEQV IAEAIRKKTR SMLLSSEQLK LCVLEYQGKY
              260 270 280 290 300
ILKVCGCDEY FLEKYPLSQY KYIRSCIMLG RMPNLMLMAK ESLYSQLPMD
              310 320 330 340 350
CFTMPSYSRR ISTATPYMNG ETSTKSLWVI NSALRIKILC ATYVNVNIRD
              360 370 380 390 400
IDKIYVRTGI YHGGEPLCDN VNTQRVPCSN PRWNEWLNYD IYIPDLPRAA
              410 420 430 440 450
RLCLSICSVK GRKGAKEEHC PLAWGNINLF DYTDTLVSGK MALNLWPVPH
              460 470 480 490 500
GLEDLLNPIG VTGSNPNKET PCLELEFDWF SSVVKFPDMS VIEEHANWSV
              510 520 530 540 550
SREAGFSYSH AGLSNRLARD NELRENDKEQ LKAISTRDPL SEITEQEKDE
              560 570 580 590 600
LWSHRHYCVT IPEILPKLLL SVKWNSRDEV AQMYCLVKDW PPIKPEQAME
              610 620 630 640 650
LLDCNYPDPM VRGFAVRCLE KYLTDDKLSQ YLIQLVQVLK YEQYLDNLLV
              660 670 680 690 700
RFLLKKALTN QRIGHFFFWH LKSEMHNKTV SQRFGLLLES YCRACGMYLK
              710 720 730 740 750
HLNRQVEAME KLINLTDILK QEKKDETQKV QMKFLVEQMR RPDFMDALQG
              760 770 780 790 800
FLSPLNPAHQ LGNLRLEECR IMSSAKRPLW LNWENPDIMS ELLFQNNEII
              810 820 830 840 850
FKNGDDLRQD MLTLQIIRIM ENIWQNQGLD LRMLPYGCLS IGDCVGLIEV
              860 870 880 890 900
VRNSHTIMQI QCKGGLKGAL QFNSHTLHQW LKDKNKGEIY DAAIDLFTRS
              910 920 930 940 950
CAGYCVATFI LGIGDRHNSN IMVKDDGQLF HIDFGHFLDH KKKKFGYKRE
              960 970 980 990 1000
RVPFVLTQDF LIVISKGAQE CTKTREFERF QEMCYKAYLA IRQHANLFIN
            1010 1020 1030 1040 1050
LFSMMLGSGM PELQSFDDIA YIRKTLALDK TEQEALEYFM KQMNDAHHGG
            1060
WTTKMDWIFH TIKQHALN
```

### NUMBERED EMBODIMENTS

1. A method of treating a patient suffering from cancer comprising administrating
   (iii) compound 1-1 and

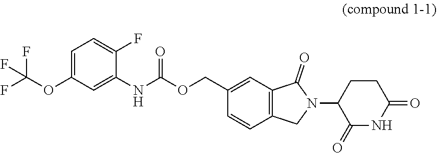 [SMILES: O=C1CCC(N2Cc3ccc(COC(=O)Nc4cc(OC(F)(F)F)ccc4F)cc3C2=O)C(=O)N1]

(iv) everolimus.
2. The method of embodiment 1, wherein the cancer is selected from the group consisting of breast cancer, lung cancer and multiple myeloma.
3. The method of embodiment 1 or 2, wherein the cancer is selected from the group consisting of breast cancer, non-small lung cancer, small lung cancer, B cell lymphoma and multiple myeloma.
4. The method of any one of embodiments 1-3, wherein the cancer has elevated expression of one or more Myc transcription factor biomarkers.
5. The method of any one of embodiments 1-4, wherein the one or more Myc transcription factor biomarkers are selected from the group consisting of: L-Myc, N-Myc, c-Myc, EIF4EBP1 and EIF4EBP2.
6. The method of any one of embodiments 1-5, wherein the one of more Myc transcription factor biomarkers are selected from the group consisting of: L-Myc and N-Myc.
7. The method of any one of embodiments 1-6, wherein the cancer exhibits a PIK3CA mutation.
8. A method of treating a patient suffering from cancer comprising administrating:
   (i) a GSPT1 degrader; and
   (ii) a compound selected from the group consisting of: a PI3K inhibitor, an Akt inhibitor, and an mTOR inhibitor.
9. The method of embodiment 1 or embodiment 8, wherein the cancer is associated with dysregulated translation.
10. The method of embodiment 8, wherein the cancer exhibits one or more PI3K-AKT-mTOR pathway gene mutations.
11. The method of embodiment 10, wherein the cancer exhibits one or more mutations selected from the group consisting of: a PIK3CA mutation, a PTEN mutation, an AKT1 mutation, a PIK3R1 mutation, and a PIK3CG mutation.
12. The method of embodiment 11, wherein the cancer exhibits a PIK3CA mutation.
13. The method of embodiment 1 or embodiment 8, wherein the cancer is selected from the group consisting of: renal angiomyolipoma, renal cell carcinoma, subependymal giant cell astrocytoma (SEGA), breast cancer, lung cancer, pancreatic cancer, and gastrointestinal (GI) cancer.
14. The method of embodiment 1 or embodiment 8, wherein the cancer is selected from the group consisting of carcinoid tumor, large cell carcinoma, uterine cancer, astrocytoma, acute myeloid leukemia, arrhythmia rhabdomyosarcoma, biliary cancer, salivary gland cancer, non-hodgkin lymphoma, B-cell lymphoma and diffuse large B-cell lymphoma.
15. The method of embodiment 8 or embodiment 13, wherein the cancer is breast cancer.
16. The method of embodiment 15, wherein the breast cancer is estrogen receptor positive breast cancer.
17. The method of embodiment 15, wherein the breast cancer a triple-negative breast cancer.
18. The method of embodiment 8 or embodiment 13, wherein the cancer is lung cancer.
19. The method of embodiment 18, wherein the lung cancer is non-small cell lung cancer.
20. The method of embodiment 19, wherein the lung cancer is a small cell lung cancer.
21. The method of any one of embodiments 8-20, wherein the cancer is a neuroendocrine cancer.
22. The method of embodiment 8 or embodiment 21, wherein the cancer is selected from the group consisting of lung neuroendocrine cancer and pancreatic neuroendocrine cancer.
23. The method of any one of embodiments 8-22, wherein the cancer is associated with tuberous sclerosis.
24. The method of embodiment 23, wherein the cancer is selected from the group consisting of renal angiomyolipoma associated with tuberous sclerosis and subependymal giant cell astrocytoma associated with tuberous sclerosis.
25. The method of embodiment 7 or embodiment 12, wherein the PIK3CA mutation is selected from the group consisting of: R38C, R38H, R88Q, P104R, G106V, R108P, de1K111, G118D, G122D, P124T, N345K, D350H, C₃₇₈R, C420R, E453Q, P539R, E542K, E542G, E542V, E545K, E545G, E545D, Q546K, Q546P, Q661K, H701P, C901F, F909L, 51008P, T1025A, T1025N, M1043I, H1047Y, H1047R, H1047L, and G1049S.
26. The method of embodiment 25, wherein the PIK3CA mutation is selected from the group consisting of E542K, E545K H1047R, H1047L.
27. The method of embodiment 8, wherein the cancer has elevated expression of one or more Myc transcription factor biomarkers selected from the group consisting of: L-Myc, N-Myc, c-Myc, EIF4EBP1 and EIF4EBP2.
28. The method of embodiment 8, wherein the GSPT1 degrader is a compound or a pharmaceutically acceptable salt or stereoisomer thereof of formula I:

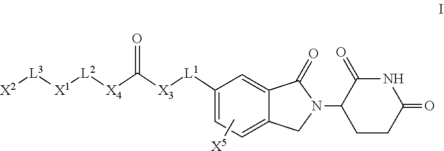

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy; or
X¹ forms together with X⁴ a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
X⁵ is H, linear or branched C₁₋₆ alkyl, —C₁₋₄ alkoxy, —CN, halogen, CF₃, CHF₂, CMeF₂, OCF₃, OCHF₂;
L¹ is a covalent bond, C₁₋₆ alkyl, which is unsubstituted or substituted with one or more of C₁₋₄ alkyl, halogen;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen.
29. The method of embodiment 8, wherein the GSPT1 degrader is a compound or a pharmaceutically acceptable salt or stereoisomer thereof of formula II,

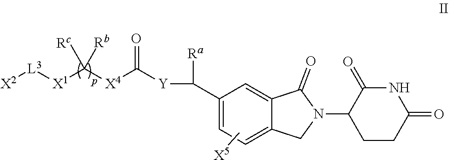

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X⁴ is —NH—;
X⁵ is H, linear or branched C₁₋₆ alkyl, —C₁₋₄ alkoxy, —CN, halogen, CF₃, CHF₂, CMeF₂, OCF₃, OCHF₂;
Y is O;
R^{a} is a H or C₁₋₄ alkyl;
R^{b}, R^{c} are independently of each other H, C₁₋₄ alkyl, preferably methyl, ethyl, or halogen, preferably F;
L³ is a covalent bond, —O—, —C₁₋₄ alkoxy or C₁₋₆ alkyl, which is unsubstituted or substituted with one or more of C₁₋₄ alkyl, halogen; and
p is 0, 1, 2.
30. The method of embodiment 8, wherein the GSPT1 degrader is a compound or a pharmaceutically acceptable salt or stereoisomer thereof of formula Va:

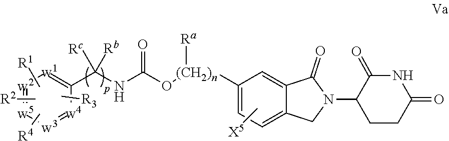

wherein
w¹, w², w³, w⁴, w⁵ are independently of each other selected from C and N, with the proviso that at least three of w¹, w², w³, w⁴, w⁵ are C;
X⁵ is H, linear or branched C₁₋₆ alkyl, —C₁₋₄ alkoxy, —CN, halogen, CF₃, CHF₂, CMeF₂, OCF₃, OCHF₂;
R¹, R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
R^{a} is H, linear or branched C₁₋₄ alkyl, R^{b}, R^{c} are independently of each other H, linear or branched C₁₋₄ alkyl; n is 1, or 2; and p is 0 or 1.
31. The method of embodiment 8, wherein the GSPT1 degrader is selected from the group consisting of
   [2-(2,6-dioxopiperidin-3-yl)-3-oxo-2,3-dihydro-1H-isoindol-5-yl]methyl N-[2-fluoro-5-(trifluoromethoxy)phenyl]carbamate or a pharmaceutically acceptable salt thereof,
   [2-(2,6-dioxopiperidin-3-yl)-3-oxo-2,3-dihydro-1H-isoindol-5-yl]methyl N-[4-fluoro-3-(trifluoromethoxy)phenyl]carbamate or a pharmaceutically acceptable salt thereof,
   [2-(2,6-dioxopiperidin-3-yl)-3-oxo-2,3-dihydro-1H-isoindol-5-yl]methyl N-[3-(difluoromethoxy)-4-fluorophenyl]carbamate or a pharmaceutically acceptable salt thereof,
   [2-(2,6-dioxopiperidin-3-yl)-3-oxo-2,3-dihydro-1H-isoindol-5-yl]methyl N-(3,5-dimethylphenyl)carbamate or a pharmaceutically acceptable salt thereof,
   [2-(2,6-dioxopiperidin-3-yl)-3-oxo-2,3-dihydro-1H-isoindol-5-yl]methyl N-[3-(trifluoromethoxy)phenyl]carbamate or a pharmaceutically acceptable salt thereof,
   [2-(2,6-dioxopiperidin-3-yl)-3-oxo-2,3-dihydro-1H-isoindol-5-yl]methyl N-(3-chloro-4-methylphenyl)carbamate or a pharmaceutically acceptable salt thereof, and
32. The method of embodiment 8, wherein the compound of (ii) is an mTOR inhibitor.
33. The method of embodiment 32, wherein the mTOR inhibitor is selected from the group consisting of:
   (1R,9S,12S,15R,16E,18R,19R,21R,23S,24E,26E,28E,30S,32S,35R)-1,18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone or a pharmaceutically acceptable salt thereof, and
   [5-[2-[(2R,6S)-2,6-dimethylmorpholin-4-yl]-4-morpholin-4-ylpyrido[2,3-d]pyrimidin-7-yl]-2-methoxyphenyl]methanol or a pharmaceutically acceptable salt thereof.
34. The method of embodiment 33, wherein the mTOR inhibitor is (1R,9S,12S,15R,16E,18R,19R,21R,23S,24E,26E,28E,30S,32S,35R)-1,18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone or a pharmaceutically acceptable salt (everolimus).
35. The method of embodiment 8, wherein the compound of (ii) is a PI3K inhibitor.
36. The method of embodiment 8, wherein the compound of (ii) is a PIK3CA inhibitor.
37. The method of embodiment 36, wherein the PIK3CA inhibitor is selected from the group consisting of:
   (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide or a pharmaceutically acceptable salt thereof,
   5-[7-methyl-6-[(4-methylsulfonylpiperazin-1-yl)methyl]-4-morpholin-4-ylthieno[3,2-d]pyrimidin-2-yl]pyrimidin-2-amine or a pharmaceutically acceptable salt thereof,
   ethyl 6-[5-(benzenesulfonamido)pyridin-3-yl]imidazo[1,2-a]pyridine-3-carboxylate or a pharmaceutically acceptable salt thereof, and
   [6-(2-amino-1,3-benzoxazol-5-yl)imidazo[1,2-a]pyridin-3-yl]-morpholin-4-ylmethanone or a pharmaceutically acceptable salt thereof.
38. The method of embodiment 37, wherein the PIK3CA inhibitor is (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide (alpelisib).
39. The method of embodiment 8, wherein the compound of (ii) is an Akt inhibitor.
40. The method of embodiment 8 or 39, wherein the Akt inhibitor is selected from the group consisting of
   4-amino-N-[(1S)-1-(4-chlorophenyl)-3-hydroxypropyl]-1-(7H-pyrrolo[2,3-d]pyrimidin-4-yl)piperidine-4-carboxamide or a pharmaceutically acceptable salt thereof, and
   4-[[(1S)-2-(azetidin-1-yl)-1-[4-chloro-3-(trifluoromethyl)phenyl]ethyl]amino]quinazoline-8-carboxamide or a pharmaceutically acceptable salt thereof.
41. The method of embodiment 8, wherein the GSPT1 degrader is [2-(2,6-dioxopiperidin-3-yl)-3-oxo-2,3-dihydro-1H-isoindol-5-yl]methyl N-[2-fluoro-5-(trifluoromethoxy)phenyl]carbamate or a pharmaceutically acceptable salt thereof, and the compound of (ii) is (1R,9S,12S,15R,16E,18R,19R,21R,23S,24E,26E,28E,30S,32S,35R)-1,18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone or a pharmaceutically acceptable salt thereof.
42. The method of embodiment 8, wherein the GSPT1 degrader is [2-(2,6-dioxopiperidin-3-yl)-3-oxo-2,3-dihydro-1H-isoindol-5-yl]methyl N-[2-fluoro-5-(trifluoromethoxy)phenyl]carbamate or a pharmaceutically acceptable salt thereof, and the compound of (ii) is (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide or a pharmaceutical acceptable salt thereof (Alpelisib).
43. The method of embodiment 8, wherein the GSPT1 degrader and the compound of (ii) are administrated together.
44. The method of embodiment 8, wherein the GSPT1 degrader is administrated prior to administration of the compound of (ii).
45. The method of embodiment 8, wherein the compound of (ii) is administrated prior to administration of the GSPT1 degrader.
46. The method of embodiment 8, wherein the method further comprises administrating a calcium supplement to the patient.
47. The method of embodiment 8, wherein the cancer has elevated expression of one or more Myc transcription factor biomarkers.
48. The method of embodiment 47, wherein the one or more Myc transcription factor biomarkers are selected from the group consisting of: L-Myc, N-Myc, c-Myc, EIF4EBP1 and EIF4EBP2.
49. The method of embodiment 48, wherein the one of more Myc transcription factor biomarkers are selected from the group consisting of: L-Myc and N-Myc.
50. The method of any one of embodiments 1-49, wherein the method further comprises a step of determining the expression level of one or more Myc transcription factor biomarkers in a biological sample obtained from the patient.
51. The method of embodiment 50, wherein the step of determining is performed prior to the steps of administration.
52. The method of embodiment 50, wherein the biological sample comprises tumor cells or tumor nucleic acid.
53. The method of embodiment 50, wherein the step of determining comprises acquiring data.
54. The method of embodiment 50, wherein the step of determining comprises obtaining a biological sample and measuring expression or having a biological sample obtained and having expression measured.
55. The method of embodiment 50, wherein the tumor nucleic acid is tumor DNA or tumor RNA.
56. The method of embodiment 50, wherein the step of determining expression level comprising measuring the copy number a gene encoding a Myc transcription factor biomarker.
57. The method of embodiment 50, wherein the one or more Myc transcription factor biomarkers are selected from the group consisting of: L-Myc, N-Myc, c-Myc, EIF4EBP1 and EIF4EBP2.
58. The method of embodiment 57, wherein the one of more Myc transcription factor biomarkers are selected from the group consisting of: L-Myc and N-Myc.
59. A method of treating cancer in a human patient, the method comprising:
   identifying a human patient as being in need of treatment for cancer;
   testing or having tested, a biological sample obtained from the patient, thereby determining that the patient's cancer exhibits with elevated expression levels of one or more Myc transcription factor biomarkers;
   selecting treatment with a GSPT1degrader and a compound of (ii) selected from the group of consisting of a PI3K inhibitor, an Akt inhibitor, and an mTOR inhibitor for the cancer that exhibits elevated expression level of one or more Myc transcription factor biomarkers; and
   treating the patient with a GSPT1 degrader and a compound of (ii) selected from the group of a PI3K inhibitor, an Akt inhibitor, and an mTOR inhibitor
60. A method of treating cancer in a human patient, the method comprising:
   identifying a human patient having a cancer that is associated with elevated expression levels of one or more Myc transcription factor biomarkers;
   selecting treatment with a GSPT1 degrader and a compound of (ii) selected from the group consisting of: a PI3K inhibitor, an Akt inhibitor, and an mTOR inhibitor for the cancer that exhibits elevated expression level of one or more Myc transcription factor biomarkers; and
   treating the patient with a GSPT1 degrader and a compound of (ii) selected from the group of a PI3K inhibitor, an Akt inhibitor, and an mTOR inhibitor
61. A method of treating cancer in a human patient, the method comprising
   identifying a human patient as being in need of treatment for cancer;
   testing or having tested a biological sample obtained from the patient, thereby determining that the patient has elevated expression level of one or more Myc transcription factor biomarkers;
   selecting treatment with a GSPT1 degrader and a compound of (ii) selected from the group consisting of: a PI3K inhibitor, an Akt inhibitor, and an mTOR inhibitor for the cancer that exhibits elevated expression level of one or more Myc transcription factor biomarkers; and
   treating the patient with a GSPT1 degrader and a compound of (ii) selected from the group of a PI3K inhibitor, an Akt inhibitor, and an TOR inhibitor.
62. A method of treating a patient suffering from cancer comprising administrating: a GSPT1 degrader; and (ii) a compound of (ii) selected from the group consisting of a PI3K inhibitor, an Akt inhibitor, and an mTOR inhibitor, wherein a biological sample obtained from the patient has previously been tested and the testing determined that the cancer has elevated expression level of one or more Myc transcription factor biomarkers.
63. The method of embodiment 59 or 61, wherein the step of determining comprises obtaining a biological sample and measuring expression or having a biological sample obtained and having expression measured.
64. The method of embodiment 59 or 61, wherein the biological sample comprises tumor cells or tumor nucleic acid.
65. The method of embodiment 64, wherein the tumor nucleic acid is tumor DNA or tumor RNA.
66. The method of embodiment 59 or 61, wherein the step of determining expression level comprising measuring the copy number a gene encoding a Myc transcription factor biomarker.
67. The method of any one of embodiments 59-66, wherein the one or more Myc transcription factor biomarkers are selected from the group consisting of: L-Myc, N-Myc, c-Myc, EIF4EBP1 and EIF4EBP2.
68. The method of embodiment 67, wherein the one of more Myc transcription factor biomarkers are selected from the group consisting of: L-Myc and N-Myc.
69. The method of any of embodiments 47-68, wherein the expression level of the one or more Myc transcription factor biomarkers is elevated relative to a reference level for that biomarker.
70. The method of embodiment 69, wherein the expression level is at least 5%, 10%, 15%, 20% or 25% greater than the reference level.
71. The method of embodiment 28, wherein X³ is —O—.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a patient suffering from cancer comprising administering to the patient
(i) a compound or a pharmaceutically acceptable salt or stereoisomer thereof of formula Va: 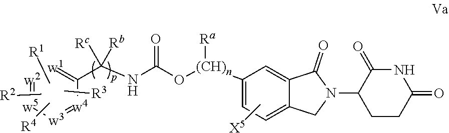
wherein
w¹, w², w³, w⁴, w⁵ are independently of each other selected from C and N, with the proviso that at least three of w¹, w², w³, w⁴, w⁵ are C;
X⁵ is H, linear or branched C₁₋₆ alkyl, —C₁₋₄ alkoxy, —CN, halogen, CF₃, CHF₂, CMeF₂, OCF₃, OCHF₂;
R¹, R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
R^{a} is H, linear or branched C₁₋₄ alkyl, R^{b}, R^{c} are independently of each other H, linear or branched C₁₋₄ alkyl; n is 1, or 2; and p is 0 or 1; and (ii) everolimus.

2. (canceled)

3. The method of claim 1, wherein the cancer is selected from the group consisting of breast cancer, non-small lung cancer, small lung cancer, B cell lymphoma and multiple myeloma.

4. The method of claim 1, wherein the cancer has elevated expression of one or more Myc transcription factor biomarkers, wherein the one or more Myc transcription factor biomarkers are selected from the group consisting of: L-Myc, N-Myc, c-Myc, EIF4EBP1 and EIF4EBP2.

5.-7. (canceled)

8. A method of treating a patient suffering from cancer comprising administering to the patient:
(i) a GSPT1 degrader; and
(ii) a compound selected from the group consisting of: a PI3K inhibitor, an Akt inhibitor, and an mTOR inhibitor,
wherein the GSPT1 degrader is a compound or a pharmaceutically acceptable salt or stereoisomer thereof of formula I: 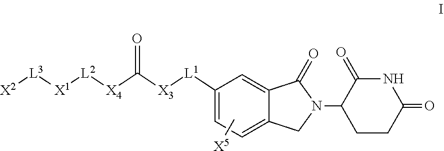
wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy; or
X¹ forms together with X⁴ a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
X⁵ is H, linear or branched C₁₋₆ alkyl, —C₁₋₄ alkoxy, —CN, halogen, CF₃, CHF₂, CMeF₂, OCF₃, OCHF₂;
L¹ is a covalent bond, C₁₋₆ alkyl, which is unsubstituted or substituted with one or more of C₁₋₄ alkyl, halogen;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen.

9.-12. (canceled)

13. The method of claim 8, wherein the cancer is selected from the group consisting of renal angiomyolipoma, renal cell carcinoma, subependymal giant cell astrocytoma (SEGA), breast cancer, lung cancer, pancreatic cancer, and gastrointestinal (GI) cancer.

14. The method of 8, wherein the cancer is selected from the group consisting of carcinoid tumor, large cell carcinoma, uterine cancer, astrocytoma, acute myeloid leukemia, arrhythmia rhabdomyosarcoma, biliary cancer, salivary gland cancer, non-hodgkin lymphoma, B-cell lymphoma and diffuse large B-cell lymphoma.

15.-26. (canceled)

27. The method of claim 8, wherein the cancer has elevated expression of one or more Myc transcription factor biomarkers selected from the group consisting of L-Myc, N-Myc, c-Myc, EIF4EBP1 and EIF4EBP2.

28. (canceled)

29. The method of claim 8, wherein the GSPT1 degrader is a compound or a pharmaceutically acceptable salt or stereoisomer thereof of formula II, 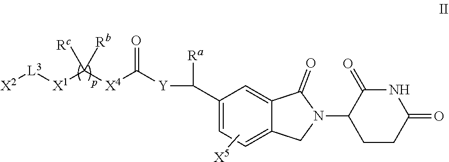
wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X⁴ is —NH—;
X⁵ is H, linear or branched C₁₋₆ alkyl, —C₁₋₄ alkoxy, —CN, halogen, CF₃, CHF₂, CMeF₂, OCF₃, OCHF₂;
Y is O;
R^{a} is a H or C₁₋₄ alkyl;
R^{b}, R^{c} are independently of each other H, C₁₋₄ alkyl, preferably methyl, ethyl, or halogen, preferably F;
L³ is a covalent bond, —O—, —C₁₋₄ alkoxy or C₁₋₆ alkyl, which is unsubstituted or substituted with one or more of C₁₋₄ alkyl, halogen; and
p is 0, 1, 2.

30. The method of claim 8, wherein the GSPT1 degrader is a compound or a pharmaceutically acceptable salt or stereoisomer thereof of formula Va: 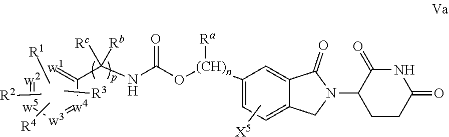
wherein
w¹, w², w³, w⁴, w⁵ are independently of each other selected from C and N, with the proviso that at least three of w¹, w², w³, w⁴, w⁵ are C;
X⁵ is H, linear or branched C₁₋₆ alkyl, —C₁₋₄ alkoxy, —CN, halogen, CF₃, CHF₂, CMeF₂, OCF₃, OCHF₂;
R¹, R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
R^{a} is H, linear or branched C₁₋₄ alkyl, R^{b}, R^{c} are independently of each other H, linear or branched C₁₋₄ alkyl; n is 1, or 2; and p is 0 or 1.

31. The method of claim 8, wherein the GSPT1 degrader is selected from the group consisting of
[2-(2,6-dioxopiperidin-3-yl)-3-oxo-2,3-dihydro-1H-isoindol-5-yl]methyl N-[2-fluoro-5-(trifluoromethoxy)phenyl]carbamate or a pharmaceutically acceptable salt thereof,
[2-(2,6-dioxopiperidin-3-yl)-3-oxo-2,3-dihydro-1H-isoindol-5-yl]methyl N-[4-fluoro-3-(trifluoromethoxy)phenyl]carbamate or a pharmaceutically acceptable salt thereof,
[2-(2,6-dioxopiperidin-3-yl)-3-oxo-2,3-dihydro-1H-isoindol-5-yl]methyl N-[3-(difluoromethoxy)-4-fluorophenyl]carbamate or a pharmaceutically acceptable salt thereof,
[2-(2,6-dioxopiperidin-3-yl)-3-oxo-2,3-dihydro-1H-isoindol-5-yl]methyl N-(3,5-dimethylphenyl)carbamate or a pharmaceutically acceptable salt thereof,
[2-(2,6-dioxopiperidin-3-yl)-3-oxo-2,3-dihydro-1H-isoindol-5-yl]methyl N-[3-(trifluoromethoxy)phenyl]carbamate or a pharmaceutically acceptable salt thereof, and
[2-(2,6-dioxopiperidin-3-yl)-3-oxo-2,3-dihydro-1H-isoindol-5-yl]methyl N-(3-chloro-4-methylphenyl)carbamate or a pharmaceutically acceptable salt thereof.

32.-33. (canceled)

34. The method of claim 8, wherein the compound of (ii) is (1R,9S,12S,15R,16E,18R,19R,21R,23S,24E,26E,28E,30S,32S,35R)-1,18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone or a pharmaceutically acceptable salt thereof (everolimus).

35.-37. (canceled)

38. The method of claim 8, wherein the compound of (ii) is (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide or a pharmaceutically acceptable salt thereof (alpelisib).

39. (canceled)

40. The method of claim 8, wherein the compound of (ii) is selected from
4-amino-N-[(1S)-1-(4-chlorophenyl)-3-hydroxypropyl]-1-(7H-pyrrolo[2,3-d]pyrimidin-4-yl)piperidine-4-carboxamide or a pharmaceutically acceptable salt thereof (Capivasertib), and
4-[[(1S)-2-(azetidin-1-yl)-1-[4-chloro-3-(trifluoromethyl)phenyl]ethyl]amino]quinazoline-8-carboxamide or a pharmaceutically acceptable salt thereof (M2698).

41.-42. (canceled)

43. The method of claim 8, wherein the GSPT1 degrader and the compound of (ii) are administered together.

44. The method of claim 8, wherein the GSPT1 degrader is administered prior to administration of the compound of (ii).

45. The method of claim 8, wherein the compound of (ii) is administered prior to administration of the GSPT1 degrader.

46.-59. (canceled)

60. A method of treating cancer in a human patient, the method comprising:
identifying a human patient having a cancer that is associated with elevated expression levels of one or more Myc transcription factor biomarkers;
selecting treatment with a GSPT1 degrader and a compound of (ii) selected from the group consisting of: a PI3K inhibitor, an Akt inhibitor, and an mTOR inhibitor for the cancer that exhibits elevated expression level of one or more Myc transcription factor biomarkers; and
treating the patient with a GSPT1 degrader and a compound of (ii) selected from the group of a PI3K inhibitor, an Akt inhibitor, and an mTOR inhibitor

61.-66. (canceled)

67. The method of claim 60, wherein the one or more Myc transcription factor biomarkers are selected from the group consisting of: L-Myc, N-Myc, c-Myc, EIF4EBP1 and EIF4EBP2.

68.-71. (canceled)

72. The method of claim 1, wherein the compound of formula Va is compound 1-1 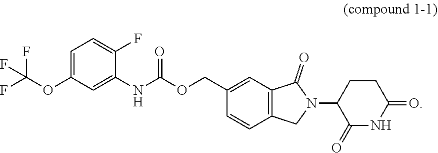 [SMILES: O=C1CCC(N2Cc3ccc(COC(=O)Nc4cc(OC(F)(F)F)ccc4F)cc3C2=O)C(=O)N1]
(ii)


SEQUENCE LISTING (1 sequence)

SEQ ID NO: 1 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN